(19) United States
(12) Patent Application Publication
(10) Pub. No.: US 2024/0076360 A1
(43) Pub. Date: Mar. 7, 2024

(54) HUMANIZED ANTI-C5 ANTIBODIES AND FACTOR H FUSION PROTEINS AND USES THEREOF

(71) Applicants: The Trustees of The University of Pennsylvania, Philadelphia, PA (US); Kira Pharmaceuticals (US) LLC, Cambridge, MA (US)

(72) Inventors: Wenchao Song, Bryn Mawr, PA (US); Takashi Miwa, Bala Cynwyd, PA (US); Sayaka Sato, Philadelphia, PA (US); Damodara Rao Gullipalli, Philadelphia, PA (US); Ping Tsui, North Potomac, MD (US); Xihua Zhu, Jiangsu (CN); Jianjun Zhang, Jiangsu (CN); Yingying Xu, Jiangsu (CN)

(21) Appl. No.: 18/259,106

(22) PCT Filed: Dec. 25, 2020
(86) PCT No.: PCT/CN2020/139556

§ 371 Date: Jun. 23, 2023

(51) Int. Cl. C07K 16/18; A61P 37/02; C07K 14/47; C12N 15/86

(52) U.S. Cl. CPC C07K 16/18; A61P 37/02; C07K 14/472; C12N 15/86; C07K 2317/14; C07K 2317/52; C07K 2317/565

(57) ABSTRACT

This invention relates to inhibition of the complement signaling using an anti-C5 antibody or fusion protein thereof. Specifically, the invention relates to methods of treating a complement-mediated disease or complement-mediated disorder in an individual by contacting the individual with an anti-C5 antibody fusion protein thereof.

### STATEMENT REGARDING FEDERALLY SPONSORED RESEARCH OR DEVELOPMENT

This invention was made with government support under NIH grant numbers AI085596 and AI117410 awarded by the National Institutes of Health (NIH). The government has certain rights in the invention.

### FIELD OF THE INVENTION

This invention relates to anti-C5 antibody, fusion protein, or fragments thereof having pH-dependent binding to C5.

### BACKGROUND OF THE INVENTION

The complement system is part of innate immunity that plays a key role in host defense. Normally the activation of complement is carefully controlled so that it does not cause autologous injury to host tissues. However, under certain situations where the regulatory mechanism is either defective (e.g. mutations in complement regulator genes) or inadequate (e.g. when there is massive autoantibody- or infection-induced complement activation that overwhelms the capacity of the regulators), severe and life-threatening autologous tissue injury by unbridled complement system may occur. Many autoimmune and inflammatory diseases are now known to be mediated by inappropriate complement activation, and there is an intense effort in the field to understand the pathogenic mechanism of various complement-mediated diseases and to develop specific anti-complement inhibitors as drugs to treat these disorders. Activated complement also has the potential to cause significant tissue injury and destruction and dysregulated complement activity has been found to be associated with a number of rare and common diseases such as paroxysmal nocturnal hemoglobinuria (PNH), atypical hemolytic uremic syndrome (aHUS), rheumatoid arthritis, age-related macular degeneration etc. Thus, anti-complement therapy is a promising way of treating these human disorders.

Complement can be activated via three different pathways, the classical, lectin and alternative pathways. All pathways converge at the C3 activation step by forming C3 convertases, C4bC2a from the classical and lectin pathways and C3bBb from the alternative pathway, respectively. C3 activation also leads to the generation of C5-cleaving enzyme complexes call C5 convertases, C4bC2aC3b and C3bBbC3b, that initiate the terminal complement activation pathway, culminating in the production of the potent pro-inflammatory mediator C5a and the membrane attack complex (MAC), C5b-9, which can cause cell lysis and death. Complement C5 is a critical protein in the terminal pathway of complement activation and is the precursor protein for generating C5a and C5b, the latter forms C5b-9 by associating with C6, C7, C8 and multiple C9 protein molecules.

A number of human inflammatory and autoimmune diseases are mediated by C5a and/or MAC, and blocking C5 activation should prevent C5a and MAC generation and be of therapeutic value. A humanized mouse anti-human C5 mAb, Eculizumab, has been used to treat a number of complement-mediated diseases including PNH and aHUS. Additionally, due to high plasma concentration of C5 and antigen target-mediated rapid removal of antibody, Eculizumab has to be administered to patients at high doses and frequency.

One of the challenges in developing drugs targeting complement proteins are their high plasma concentration and/or fast turnover. For example, the plasma concentrations of human C3 and C5 are approximately 1 mg/mL and 80 μg/mL, respectively. This means that inhibitors for such proteins need to be administered at a high dose and/or frequently. Indeed, the anti-C5 mAb drug Eculizumab is required to be given every two weeks through intravenous injection in PNH and aHUS patients at a maintenance dose of 900 mg and 1200 mg, respectively. Although a longer lasting second generation anti-C5 mAb Ravulizumab has been developed to lessen the injection frequency to every 8 weeks, the maintenance dose of Ravulizumab has increased to 3300 mg per injection. Furthermore, neither Eculizumab nor Ravulizumab was able to normalize LDH and hemoglobin levels in approximately 50% treated PNH patients. In PNH patients on standard Eculizumab therapy, breakthrough lysis are frequently observed and 20-30% patients are still transfusion-dependent. These unmet medical needs in PNH patients are related to the fact that deficiency of DAF and CD59 on affected blood cells makes them susceptible to C3 activation as well as MAC-mediated injury. Although anti-C5 mAbs, such as Eculizumab and Ravulizumab, can inhibit C5-mediated hemolysis, they do not prevent C3 activation on affected RBCs and as a result, C3b opsonization of RBCs still happens and this leads to the well-recognized phenomenon of extravascular hemolysis (EVH), a process caused by phagocytosis of C3b-opsonized RBCs in the reticuloendothelial system. In addition, studies have shown that blocking complement activation on RBCs at the C5 step has its limitation in efficacy because, if too many C5 convertases are already assembled on the cell surface, it becomes impossible to block C5 cleavage completely with mAbs of a finite avidity. This may explain the breakthrough lysis phenomenon in PNH patients treated with Eculizumab and why anti-C5 mAbs are not capable of preventing complete hemolysis of rabbit and PNH RBC cells in ex vivo assays, as both PNH and rabbit RBCs are exceedingly sensitive to C3 complement activation via the AP and can easily assemble abundant C5 convertases on the surface. Although there are ongoing efforts to target C3 activation in complement-dependent diseases, e.g. through the use of C3-inhibitory cyclic peptides or recombinant short variants of FH, such molecules have very poor pharmacokinetics and require large and frequent (e.g. daily) dosing.

All references cited herein, including patent applications, patent publications, and Genbank Accession numbers are herein incorporated by reference, as if each individual reference were specifically and individually indicated to be incorporated by reference in its entirety.

### BRIEF SUMMARY OF THE INVENTION

The present application provides an anti-C5 antibody, fusion protein, or fragments thereof having pH-dependent binding to C5.

In one aspect of the present application, there is provided an isolated antibody that specifically binds to human C5 and comprises a heavy chain variable domain (VH) and a light chain, wherein VH comprises a Y32H mutation, wherein the mutation is in reference to SEQ ID NO:1 under the AbM numbering system, and wherein the antibody binds more strongly to C5 at a neutral pH than it does at an acidic pH.

In some embodiments, the antibody further comprises a Q38R mutation in the VL, wherein the mutation is in reference to SEQ ID NO:2 under the AbM numbering system.

In some embodiments according to any one of the isolated antibodies described above, the antibody further comprises a substitution at the VL.

In some embodiments according to any one of the isolated antibodies described above, the antibody comprises a mutation in the VL selected from the group consisting of E27H, N28H, Y30H, L54H, D56H, T58H, T97H, wherein the mutation is in reference to SEQ ID NO:2 under the AbM numbering system.

In some embodiments according to any one of the isolated antibodies described above, the antibody comprises: i) a heavy chain CDR1 (“H-CDR1) comprising the amino acid sequence of SEQ ID NO:3 or a variant thereof comprising one, two, or three amino acid substitutions; ii) a heavy chain CDR2 (“H-CDR2”) comprising the amino acid sequence of SEQ ID NO:4 or a variant thereof comprising one, two, or three amino acid substitutions; iii) a heavy chain CDR3 (“H-CDR3”) comprising the amino acid sequence of SEQ ID NO:5 or a variant thereof comprising one, two, or three amino acid substitutions; iv) a light chain CDR1 (“L-CDR1”) comprising the amino acid sequence of SEQ ID NO:6 or a variant thereof comprising one, two, or three amino acid substitutions; v) a light chain CDR2 (“L-CDR2”) comprising the amino acid sequence of SEQ ID NO:7 or a variant thereof comprising one, two, or three amino acid substitutions; and vi) a light chain CDR3 (L-CDR3”) comprising the amino acid sequence of SEQ ID NO:8 or a variant thereof comprising one, two, or three amino acid substitutions.

In some embodiments according to any one of the isolated antibodies described above, the antibody comprises: i) a VH comprising the amino acid sequence SEQ ID NO:9 or a variant thereof that is at least about 85% identical to SEQ ID NO:9; and ii) a light chain comprising the amino acid sequence of SEQ ID NO:2 or a variant thereof that is at least about 85% identical to SEQ ID NO:2.

In some embodiments according to any one of the isolated antibodies described above, the antibody comprises: i) H-CDR1 comprising the amino acid sequence of SEQ ID NO:3; ii) H-CDR2 comprising the amino acid sequence of SEQ ID NO:4; iii) H-CDR3 comprising the amino acid sequence of SEQ ID NO:5; iv) L-CDR1 comprising the amino acid sequence of SEQ ID NO:6; v) L-CDR2 comprising the amino acid sequence of SEQ ID NO:7; and vi) L-CDR3 comprising the amino acid sequence of SEQ ID NO:8.

In some embodiments according to any one of the isolated antibodies described above, the antibody comprises: i) a VH comprising the amino acid sequence SEQ ID NO:9; and ii) a VL comprising the amino acid sequence of SEQ ID NO:2.

In some embodiments according to any one of the isolated antibodies described above, the antibody comprises: i) a VH comprising the amino acid sequence SEQ ID NO:9; and ii) a VL comprising the amino acid sequence of SEQ ID NO:18.

In some embodiments according to any one of the isolated antibodies described above, the antibody comprises: i) H-CDR1 comprising the amino acid sequence of SEQ ID NO:3; ii) H-CDR2 comprising the amino acid sequence of SEQ ID NO:4; iii) H-CDR3 comprising the amino acid sequence of SEQ ID NO:5; iv) L-CDR1 comprising the amino acid sequence of SEQ ID NO:11, SEQ ID NO:12, or SEQ ID NO:13; v) L-CDR2 comprising the amino acid sequence of SEQ ID NO:7; and vi) L-CDR3 comprising the amino acid sequence of SEQ ID NO:8.

In some embodiments according to any one of the isolated antibodies described above, the antibody comprises: i) a VH comprising the amino acid sequence SEQ ID NO:9; and ii) a VL comprising the amino acid sequence of SEQ ID NO:19, SEQ ID NO:20, or SEQ ID NO:21.

In some embodiments according to any one of the isolated antibodies described above, the antibody comprises: i) H-CDR1 comprising the amino acid sequence of SEQ ID NO:3; ii) H-CDR2 comprising the amino acid sequence of SEQ ID NO:4; iii) H-CDR3 comprising the amino acid sequence of SEQ ID NO:5; iv) L-CDR1 comprising the amino acid sequence of SEQ ID NO:6; v) L-CDR2 comprising the amino acid sequence of SEQ ID NO:14, SEQ ID NO:15, or SEQ ID NO:16; and vi) L-CDR3 comprising the amino acid sequence of SEQ ID NO:8.

In some embodiments according to any one of the isolated antibodies described above, the antibody comprises: i) a VH comprising the amino acid sequence SEQ ID NO:9; and ii) a VL comprising the amino acid sequence of SEQ ID NO:22, SEQ ID NO:23, or SEQ ID NO:24.

In some embodiments according to any one of the isolated antibodies described above, the antibody comprises: i) H-CDR1 comprising the amino acid sequence of SEQ ID NO:3; ii) H-CDR2 comprising the amino acid sequence of SEQ ID NO:4; iii) H-CDR3 comprising the amino acid sequence of SEQ ID NO:5; iv) L-CDR1 comprising the amino acid sequence of SEQ ID NO:6; v) L-CDR2 comprising the amino acid sequence of SEQ ID NO:7; and vi) L-CDR3 comprising the amino acid sequence of SEQ ID NO:17.

In some embodiments according to any one of the isolated antibodies described above, the antibody comprises: i) a VH comprising the amino acid sequence SEQ ID NO:9; and ii) a VL comprising the amino acid sequence of SEQ ID NO:25.

In some embodiments according to any one of the isolated antibodies described above, the antibody is selected from the group consisting of: a full length antibody, Fab, Fab′, F(ab)2, F(ab′)2, and scFv.

In some embodiments according to any one of the isolated antibodies described above, the antibody further comprises an Fc region. In some embodiments, the Fc region comprises an IgG4 sequence. In some embodiments, the Fc region comprises the amino acid sequence of SEQ ID NO:26 or a variant thereof. In some embodiments, the Fc region further comprises one or more mutations selected from the group consisting of S228P, M428L, and N434A, wherein the mutations are relative to SEQ ID NO:26 under the EU numbering system. In some embodiments, the Fc region comprises mutations S228P, M428L, and N434A. In some embodiments, the Fc region comprises amino acid sequence of SEQ ID NO:27.

In some embodiments according to any one of the isolated antibodies described above, the antibody has a low-pH dissociation factor of between about 20% to about 80%. In some embodiments according to any one of the isolated antibody described above, the antibody has a neutral-pH dissociation factor of between about 0% to about 12%. In some embodiments according to any one of the isolated antibody described above, the ratio of low-pH dissociation to neutral-pH dissociation of the antibody is 4 or more.

In some embodiments according to any one of the isolated antibodies described above, the antibody inhibits the cleavage of human C5 into fragments C5a and C5b.

In some embodiments according to any one of the isolated antibodies described above, the antibody has a serum half-life in humans that is at least about 25 days.

In some embodiments according to any one of the isolated antibodies described above, the antibody is manufactured in CHO cells.

In some embodiments according to any one of the isolated antibodies described above, the antibody cross-reacts with a cyno monkey C5.

In some embodiments, there is provided a fusion polypeptide comprising any one of the isolated antibodies described above, wherein the antibody fused to a factor H polypeptide or fragment thereof. In some embodiments, the antibody is fused at the N-terminus of the factor H polypeptide or fragment thereof. In some embodiments, the antibody is fused at the C-terminus of the factor H polypeptide or fragment thereof. In some embodiments according to any one of the fusion peptide described above, the factor H polypeptide or fragment thereof comprises SCR1-5 domains of factor H.

In some embodiments, there is provided a nucleic acid encoding any one of the antibodies or the fusion polypeptides described above. In some embodiments, there is provided a vector comprising the nucleic acid encoding any one of the antibodies or the fusion polypeptides described above. In some embodiments, there is provided a host cell comprising the vector comprising the nucleic acid encoding any one of the antibodies or the fusion polypeptides described above. Exemplary nucleic acid sequences are set for in SEQ ID Nos: 33-62. In some embodiments, the nucleic acid comprises the sequence of any one of SEQ ID Nos:33-62.

In some embodiments, there is provided a method of producing the antibodies or the fusion polypeptides described above, comprising culturing the host cell comprising the vector comprising the nucleic acid encoding any one of the antibodies or the fusion polypeptides described above under a condition sufficient to allow expression by cell of the antibody or fusion polypeptide encoded by the nucleic acid.

Also provided is a pharmaceutical composition comprising any one of the antibodies or the fusion polypeptides described above and a pharmaceutically acceptable carrier.

Another aspect of the present application provides a method of treating an individual having a complement-associated disease or condition, comprising administering to the individual an effective amount of any one of the pharmaceutical compositions described above. In some embodiments, the disease or condition is selected from the group consisting of: macular degeneration (MD), age-related macular degeneration (AMD), ischemia reperfusion injury, arthritis, rheumatoid arthritis, lupus, ulcerative colitis, stroke, post-surgery systemic inflammatory syndrome, asthma, allergic asthma, chronic obstructive pulmonary disease (COPD), paroxysmal nocturnal hemoglobinuria (PNH) syndrome, myasthenia gravis, neuromyelitis optica, (NMO), multiple sclerosis, delayed graft function, antibody-mediated rejection, atypical hemolytic uremic syndrome (aHUS), central retinal vein occlusion (CRVO), central retinal artery occlusion (CRAO), epidermolysis bullosa, sepsis, organ transplantation, inflammation (including, but not limited to, inflammation associated with cardiopulmonary bypass surgery and kidney dialysis), C3 glomerulopathy, membranous nephropathy, IgA nephropathy, glomerulonephritis (including, but not limited to, anti-neutrophil cytoplasmic antibody (ANCA)-mediated glomerulonephritis, lupus nephritis, and combinations thereof), ANCA-mediated vasculitis, Shiga toxin induced HUS, and antiphospholipid antibody-induced pregnancy loss, or any combinations thereof.

Another aspect of the present application provides a method of reducing the activity of a complement system in an individual, comprising administering to the individual an effective amount of any one of the pharmaceutical compositions described above.

These and other aspects and advantages of the present invention will become apparent from the subsequent detailed description and the appended claims. It is to be understood that one, some, or all of the properties of the various embodiments described herein may be combined to form other embodiments of the present invention.

### BRIEF DESCRIPTION OF THE DRAWINGS

FIG. 1 depicts the association and dissociation of C5 from various anti-C5 antibodies using Bio-Layer Interferometry (BLI) technology. Association occurred at pH 7.4 and dissociation occurred either at pH 7.4 (top curves) or pH 5.8 (bottom curves).

FIG. 2 depicts the percentage of C5 dissociation from peak C5 binding for various anti-C5 antibodies at dissociation pH of 7.4 (filled bars) and 5.8 (open bars), using Bio-Layer Interferometry (BLI) technology.

FIG. 3 depicts the association and dissociation of human C5 from Y32H/Q38R/L54H and two benchmark anti-C5 antibodies (Eculizumab and ALXN1210). Association occurred at pH 7.4 and dissociation occurred at pH 7.4 (top curves) or 5.8 (bottom curves). The concentration of human C5 is 40 nM. Curves are in duplicate. Assay was performed using a machine based on Bio-Layer Interferometry (BLI) technology.

FIG. 4 depicts Y32H/Q38R/L54H, Eculizumab and ALXN1210 binding to and dissociation from human C5 at pH 7.4 at different C5 concentrations. Bindings to immobilized antibodies were measured using various human C5 concentrations (top to bottom: 40, 20, 10, 5, 2.5, 1.25 and 0.625 nM) and using a machine based on Bio-Layer Interferometry (BLI) technology.

FIG. 5 depicts inhibition of sensitized sheep RBC lysis with 50% normal human serum (NHS) by anti-C5 antibodies at different antibody concentration levels.

FIG. 6 depicts the levels of human IgG4 determined by ELISA in the plasma of C5-humanized SCID/human FcRn-transgenic mice at various time points after injection of representative anti-C5 antibodies (40 mg/kg).

FIG. 7 depicts the relative chicken RBC hemolytic activity of sera collected from C5-humanized SCID/human FcRn-transgenic mice at different time points, mixed with C5-depleted normal human serum. Y32H/Q38R, Y32H/Q38R/L54H, Y32H/Q38R/D56H Y32H/Q38R/T97H mAbs were tested in this assay by I.V. injection (40 mg/kg).

FIG. 8 depicts the levels of human C5 determined by ELISA in the plasma of C5-humanized SOD/human FcRn-transgenic mice at various time points after injection of representative anti-C5 antibodies.

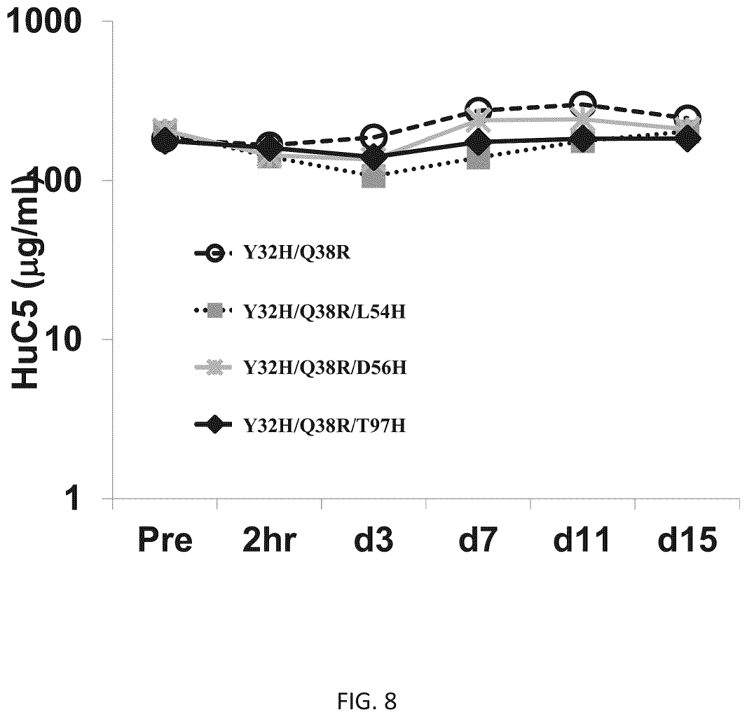

FIG. 9 depicts testing of cross-species reactivity of Y32H/Q38R/L54H and Ultomiris with plasma C5 (panels A and B) and inhibition of Cynomolgus monkey serum-induced hemolysis of sensitized sheep RBC by Y32H/Q38R/L54H mAb at high concentrations.

FIG. 10 depicts the complement activation pathways, showing the three pathways by which complement is activated. All pathways converge at the C3 activation step. Activation of C3 leads to C5 activation and formation of C5a and C5b-9. C5 activation can be inhibited by anti-C5 mAbs whereas an anti-C5 mAb and factor H (FH) fusion protein can inhibit both C3 and C5 activations steps.

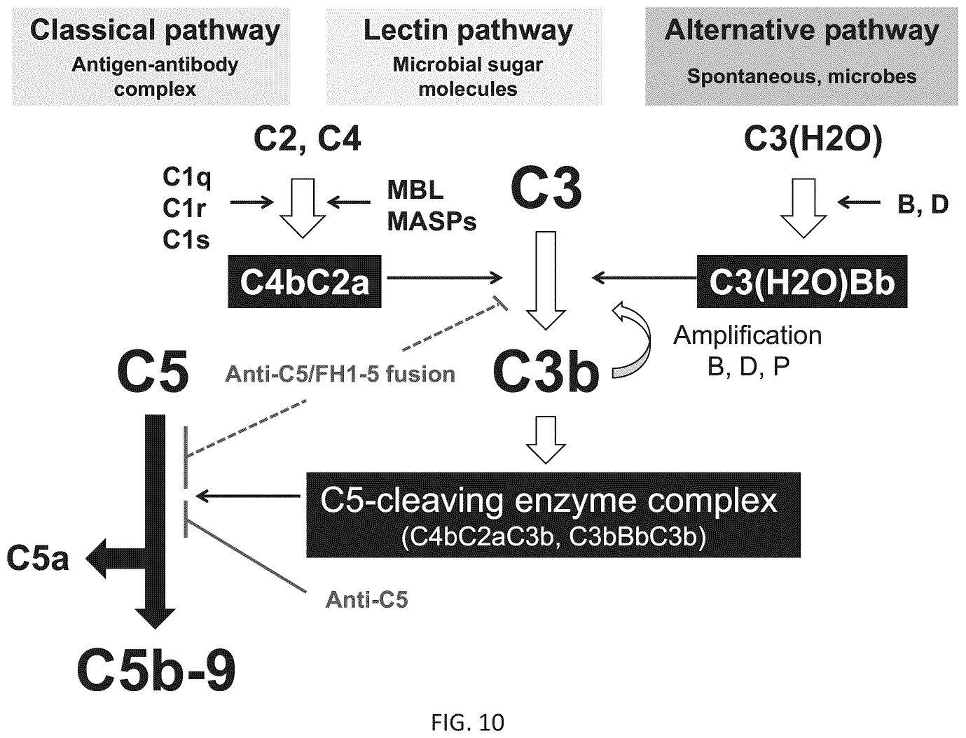

FIG. 11 shows a schematic representation of factor H (FH) domains and the construction of anti-C5 antibody-factor H fusion protein. FH is composed 20 short consensus repeat (SCR) domains but its complement regulating activity is located at SCR1-5. The fusion protein is constructed by attaching FH SCR1-5 to the C-terminal end of Fc domain of anti-C5 mAb.

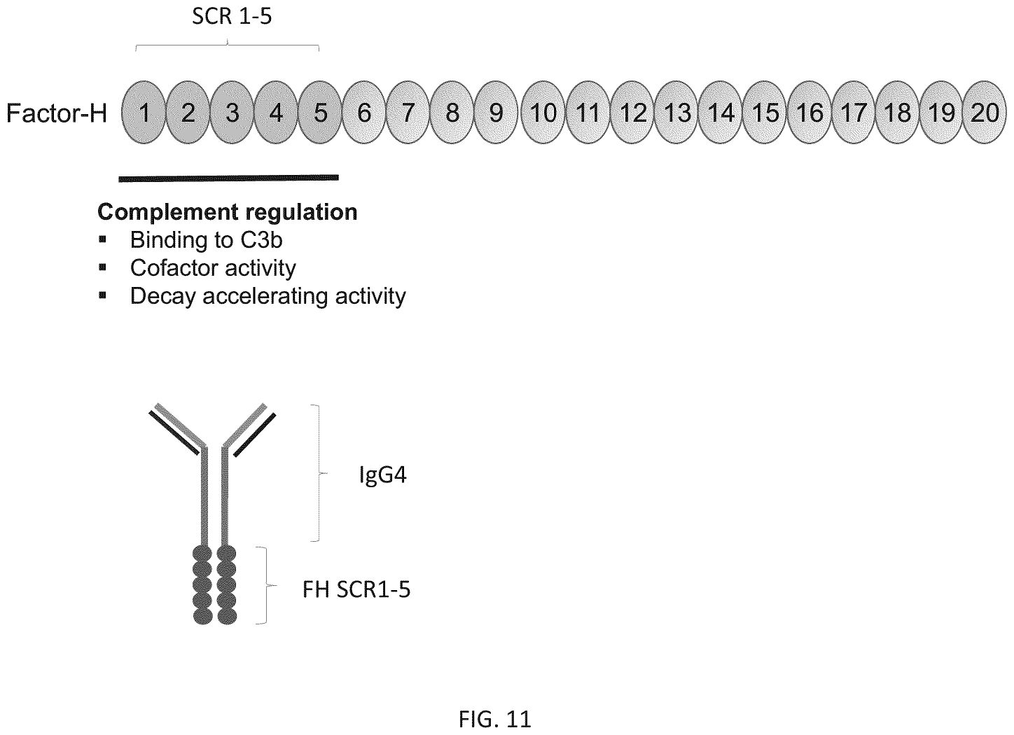

FIG. 12 depicts the association and dissociation of C5 from various anti-C5 antibody factor H fusion proteins using Bio-Layer Interferometry (BLI) technology. Association occurred at pH 7.4 and dissociation occurred either at pH 7.4 (top curves) or pH 5.8 (bottom curves).

FIG. 13 depicts the percentage of C5 dissociation from peak C5 binding for various anti-C5 antibody factor H fusion proteins at dissociation pH of 7.4 (open bars) and 5.8 (filled bars), using Bio-Layer Interferometry (BLI) technology.

FIG. 14 shows comparison of the pH-dependent dissociation kinetics of anti-C5 mAbs and anti-C5 mAb-FH fusion proteins, using Bio-Layer Interferometry (BLI) technology. Panel A shows dissociation trances at pH7.4 and pH5.8, and panel B shows the percentage of C5 dissociation from peak C5 binding for various anti-C5 antibody and antibody-FH fusion proteins at pH of 7.4 (filled bars) and 5.8 (open bars).

FIG. 15 depicts inhibition of normal human serum (50%)-mediated lysis of sensitized sheep RBC by anti-C5 antibody-factor H fusion proteins at different antibody concentration levels.

FIG. 16 depicts the inhibitory activity on human alternative pathway complement-mediated rabbit RBC lysis by Y32H/Q38R anti-C5 mAb, Y32H/Q38R anti-C5 mAb-FH SCR1-5 fusion protein, Eculizumab and Ravulizumab. Hemolytic assay was performed in Mg++-EGTA buffer using 25% normal human serum (NHS).

FIG. 17 depicts the result of FACS analysis of C3b deposition on rabbit RBC that were protected from lysis in the experiment shown in FIG. 16. Only RBC cells rescued from lysis by Y32H/Q38R anti-C5 mAb-FH SCR1-5 fusion protein were found to have drug concentration-dependent inhibition of C3b deposition.

FIG. 18 depicts the levels of human IgG determined by ELISA in the plasma of C5 humanized SCID/human FcRn-transgenic mice at various time points after injection of Eculizumab, Ravulizumab and Y32H/Q38R-Factor H SCR1-5 fusion protein (i.v. at 40 mg/kg).

FIG. 19 depicts the levels of human C5 determined by MASA in the plasma of C5 humanized. SCID/human FcRn-transgenic mice at various time points after injection of Eculizumab, Ravulizumab and Y32H/Q38R-Factor H SCR1-5 fusion protein (i.v. at 40 mg/kg).

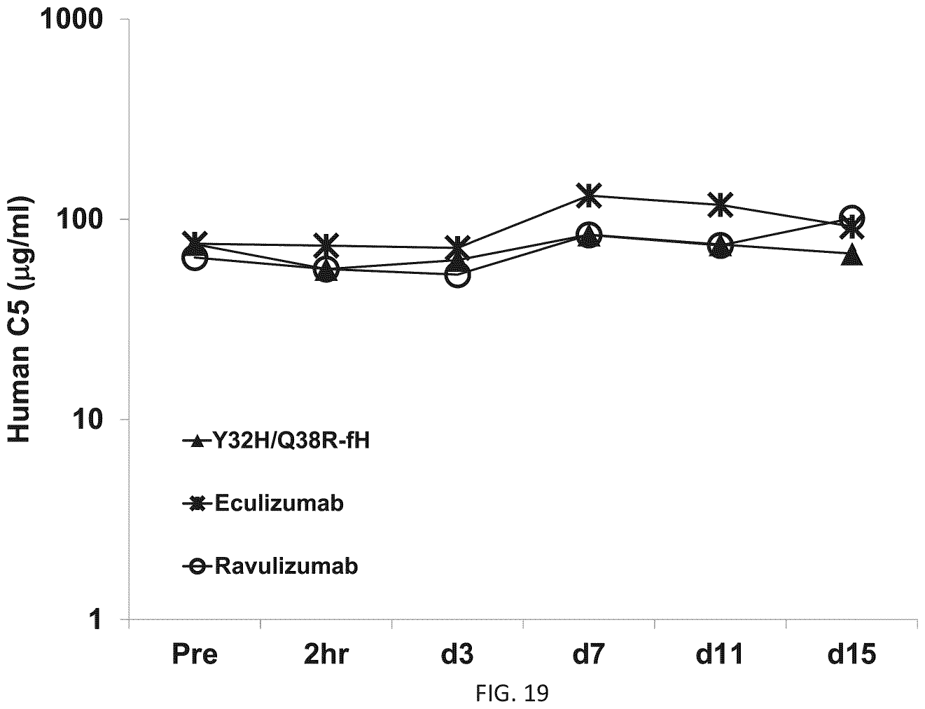

FIG. 20 depicts the pharmacodynamics properties of Y32H/Q38R-FH SCR1-5 fusion protein in comparison to Eculizumab and Ravulizumab using a human alternative pathway complement-mediated rabbit red blood cell lysis test by a mixture of C5-depleted normal human serum (NHS) and mouse serum collected at various time points after antibody treatment.

FIG. 21 depicts relative inhibitory activity on acidified human serum-mediated PNH red blood cell lysis by Eculizumab, Ravulizumab and Y32H/Q38R-FH SCR1-5 fusion protein (labeled as Y32H/Q38R-FH in the figure).

FIG. 22 depicts the results of FACS analysis of C3b deposition on non-lysed PNH red blood cells from the experiment described in FIG. 21. Note normal RBCs in this patient are shown in Quadrant 1 and 2 (Q1/Q2) and PNH RBCs are shown in Quadrant 3 and 4 (Q3/Q4). C3b deposition was detected only on PNH RBCs (Q4).

### DETAILED DESCRIPTION OF THE INVENTION

The present application provides novel anti-human C5 antibodies and constructs having mutations in its VH and/or VL domains that render the antibodies pH sensitive in antigen binding. The present invention was based on the surprising finding that anti-C5 antibodies harboring certain mutations, for example the Y32H mutation in the VH domain, bind more strongly to C5 at a neutral pH (e.g., about pH 7.4; such as that found in the blood) than it does at a more acidic pH (e.g., about pH 5.8; such as that found in the endosome), thereby rendering it particularly suitable for greater persistence of administered antibody or antibody fusion protein molecules. This mutation, when combined with certain mutations in the VL domain and/or mutations in the Fc domain, produced significantly improved pharmacokinetics and pharmacodynamics, for example when assessed in C5 humanized mice on SCID/human FcRn transgenic mice. Moreover, unlike the corresponding antibody not having the specific mutations, certain mutant antibodies show cross-reactivity to C5 of cyno monkeys, suggesting that the mutations not only alter pH sensitivity of the antibodies, but also binding properties of the antibodies.

The present application thus in one aspect provides an isolated antibody that specifically binds to human C5 and comprises a heavy chain variable domain (VH) and a light chain variable domain (VL), wherein VH comprises a Y32H mutation, and wherein the antibody binds more strongly to C5 at a neutral pH than it does at an acidic pH. Also provided are fusion constructs comprising the pH sensitive anti-C5 antibody and a second effector molecule (such as a complement inhibitor, for example factor H (CFH)).

In another aspect, there are provided methods of inhibiting complement activation and/or methods of treating diseases (such as complement associated diseases) by administering any one or more of the pH sensitive anti-C5 antibodies or constructs thereof.

In another aspect, there are provided exemplary nucleic acids encoding any one or more of the pH sensitive anti-C5 antibodies or constructs thereof, as well as vectors or host cells comprising such nucleic acids. Methods of making the pH sensitive anti-C5 antibodies are also described.

### I. Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, “treatment” or “treating” is an approach for obtaining beneficial or desired results including clinical results. For purposes of this application, beneficial or desired clinical results include, but are not limited to, one or more of the following: decreasing one more symptoms resulting from the disease, diminishing the extent of the disease, stabilizing the disease (e.g., preventing or delaying the worsening of the disease), preventing or delaying the spread of the disease, preventing or delaying the occurrence or recurrence of the disease, delay or slowing the progression of the disease, ameliorating the disease state, providing a remission (whether partial or total) of the disease, decreasing the dose of one or more other medications required to treat the disease, delaying the progression of the disease, increasing the quality of life, and/or prolonging survival. Also encompassed by “treatment” is a reduction of pathological consequence of the disease. The methods of the present application contemplate any one or more of these aspects of treatment.

The terms “effective amount” and “pharmaceutically effective amount” as used herein refer to a sufficient amount of an agent to provide the desired biological result. That result can be reduction and/or alleviation of the signs, symptoms, or causes of a disease or disorder, or any other desired alteration of a biological system.

As used herein, the terms “patient,” “subject,” “individual,” and the like are used interchangeably herein, and refer to any animal, in some embodiments a mammal, and in some embodiments a human, having a complement system, including a human in need of therapy for, or susceptible to, a condition or its sequelae. The individual may include, for example, dogs, cats, pigs, cows, sheep, goats, horses, rats, monkeys, and mice and humans. In some embodiments, the individual is a human.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody” may refer an immunoglobulin molecule or a fragment thereof which is able to specifically bind to a specific epitope of an antigen (including the basic 4-chain antibody unit). Antibodies can be intact immunoglobulins derived from natural sources, or from recombinant sources and can be immunoreactive portions of intact immunoglobulins. The antibodies in the present invention may exist in a variety of forms including, for example, polyclonal antibodies, monoclonal antibodies, intracellular antibodies (“intrabodies”), antigen-binding fragments (such as Fv, Fab, Fab′, F(ab)2 and F(ab′)2), as well as single chain antibodies (scFv), heavy chain antibodies, such as camelid antibodies, and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antigen-binding fragment” as used herein refers to an antibody fragment including, for example, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain Fv (scFv), an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment (e.g., a parent scFv) binds. In some embodiments, an antigen-binding fragment may comprise one or more CDRs from a particular human antibody grafted to a framework region from one or more different human antibodies.

“Fv” is the minimum antibody fragment, which contains a complete antigen-recognition and -binding site. This fragment consists of a dimer of one heavy- and one light-chain variable region domain in tight, non-covalent association. From the folding of these two domains emanate six hypervariable loops (3 loops each from the heavy and light chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv,” also abbreviated as “sFv” or “scFv,” are antibody fragments that comprise the VH and V_{L} antibody domains connected into a single polypeptide chain. In some embodiments, the scFv polypeptide further comprises a polypeptide linker between the VH and V_{L} domains which enables the scFv to form the desired structure for antigen binding. For a review of scFv, see Plückthun in The Pharmacology of Monoclonal Antibodies, vol. 113, Rosenburg and Moore eds., Springer-Verlag, New York, pp. 269-315 (1994).

The basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (C_{H}) for each of the α and γ chains and four C_{H} domains for μ and ε isotypes. Each L chain has at the N-terminus, a variable domain (V_{L}) followed by a constant domain at its other end. The V_{L} is aligned with the VH and the C_{L} is aligned with the first constant domain of the heavy chain (C_{H}1). Particular amino acid residues are believed to form an interface between the light chain and heavy chain variable domains. The pairing of a VH and V_{L} together forms a single antigen-binding site. For the structure and properties of the different classes of antibodies, see e.g., Basic and Clinical Immunology, 8th Edition, Daniel P. Sties, Abba I. Terr and Tristram G. Parsolw (eds), Appleton & Lange, Norwalk, Conn., 1994, page 71 and Chapter 6. The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG and IgM, having heavy chains designated α, δ, ε, γ and μ, respectively. The γ and α classes are further divided into subclasses on the basis of relatively minor differences in the C_{H} sequence and function, e.g., humans express the following subclasses: IgG1, IgG2A, IgG2B, IgG3, IgG4, IgA1 and IgA2.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

An “isolated” antibody is one that has been identified, separated and/or recovered from a component of its production environment (E.g., natural or recombinant). Preferably, the isolated polypeptide is free of association with all other components from its production environment. Contaminant components of its production environment, such as that resulting from recombinant transfected cells, are materials that would typically interfere with research, diagnostic or therapeutic uses for the antibody, and may include enzymes, hormones, and other proteinaceous or non-proteinaceous solutes. In preferred embodiments, the polypeptide will be purified: (1) to greater than 95% by weight of antibody as determined by, for example, the Lowry method, and in some embodiments, to greater than 99% by weight; (1) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or, preferably, silver stain. Isolated antibody includes the antibody in situ within recombinant cells since at least one component of the antibody's natural environment will not be present. Ordinarily, however, an isolated polypeptide or antibody will be prepared by at least one purification step.

The “variable region” or “variable domain” of an antibody refers to the amino-terminal domains of the heavy or light chain of the antibody. The variable domains of the heavy chain and light chain may be referred to as “VH” and “VL”, respectively. These domains are generally the most variable parts of the antibody (relative to other antibodies of the same class) and contain the antigen binding sites. Heavy-chain only antibodies from the Camelidae species have a single heavy chain variable region, which is referred to as “VHH”. VHH is thus a special type of VH.

The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen binding site of antibodies (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent cellular toxicity.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present application may be made by a variety of techniques, including, for example, the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2^{nd} ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The terms “full-length antibody,” “intact antibody” or “whole antibody” are used interchangeably to refer to an antibody in its substantially intact form, as opposed to an antibody fragment. Specifically full-length 4-chain antibodies include those with heavy and light chains including an Fc region. The constant domains may be native sequence constant domains (e.g., human native sequence constant domains) or amino acid sequence variants thereof. In some cases, the intact antibody may have one or more effector functions.

The term “diabodies” refers to small antibody fragments prepared by constructing sFv fragments (see preceding paragraph) with short linkers (about 5-10) residues) between the VH and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the VH and V_{L} domains of the two antibodies are present on different polypeptide chains. Diabodies are described in greater detail in, for example, EP 404,097; WO 93/11161; Hollinger et al., Proc. Natl. Acad. Sci. USA 90: 6444-6448 (1993).

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include PRIMATTZFD® antibodies wherein the antigen-binding region of the antibody is derived from an antibody produced by, e.g., immunizing macaque monkeys with an antigen of interest. As used herein, “humanized antibody” is used a subset of “chimeric antibodies.”

“Humanized” forms of non-human (e.g., murine) antibodies are chimeric antibodies that contain minimal sequence derived from non-human immunoglobulin. In some embodiments, a humanized antibody is a human immunoglobulin (recipient antibody) in which residues from an HVR (hereinafter defined) of the recipient are replaced by residues from an HVR of a non-human species (donor antibody) such as mouse, rat, rabbit or non-human primate having the desired specificity, affinity, and/or capacity. In some instances, framework (“FR”) residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The number of these amino acid substitutions in the FR is typically no more than 6 in the H chain, and in the L chain, no more than 3. The humanized antibody optionally will also comprise at least a portion of an immunoglobulin constant region (Fc), typically that of a human immunoglobulin. A suitable human acceptor antibody may be one selected from a conventional database, e.g., the KABAT database, Los Alamos database, the AbM, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanized antibodies (see, for example, EP-A-0239400 and EP-A-054951). For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

The term “donor antibody” refers to an antibody (monoclonal, and/or recombinant) which contributes the amino acid sequences of its variable regions, CDRs, or other functional fragments or analogs thereof to a first immunoglobulin partner, so as to provide the altered immunoglobulin coding region and resulting expressed altered antibody with the antigenic specificity and neutralizing activity characteristic of the donor antibody.

The term “acceptor antibody” refers to an antibody (monoclonal and/or recombinant) heterologous to the donor antibody, which contributes all (or any portion, but in some embodiments all) of the amino acid sequences encoding its heavy and/or light chain framework regions and/or its heavy and/or light chain constant regions to the first immunoglobulin partner. In certain embodiments a human antibody is the acceptor antibody.

The term “fusion protein” refers a protein created through the attachment of two or more polypeptides which originated from separate proteins. In some embodiments, fusion proteins are created using recombinant DNA technology (e.g., by attaching the nucleic acids that encode each of the parts of the fusion protein) and are typically used in biological research or therapeutics. In some embodiments, fusion proteins are created through chemical conjugation (e.g., covalent conjugation, etc.) with or without a linker between the polypeptides portion of the fusion proteins.

The term “attach” or “attached” as used herein, refers to connecting or uniting by a bond, link, force or tie in order to keep two or more components together, which encompasses either direct or indirect attachment such that, for example, where a first polypeptide is directly bound to a second polypeptide or material, and, for example, where one or more intermediate compounds (e.g., amino acids, peptides, polypeptides, etc.) are disposed between the first polypeptide and the second polypeptide or material.

“CDRs” are defined as the complementarity determining region amino acid sequences of an antibody which are the hypervariable regions of immunoglobulin heavy and light chains. See, e.g., Kabat et al., Sequences of Proteins of Immunological Interest, 4th Ed., U.S. Department of Health and Human Services, National Institutes of Health (1987). There are three heavy chain and three light chain CDRs (or CDR regions) in the variable portion of an immunoglobulin. Thus, “CDRs” as used herein refers to all three heavy chain CDRs, or all three light chain CDRs (or both all heavy and all light chain CDRs, if appropriate). The structure and protein folding of the antibody may mean that other residues are considered part of the antigen binding region and would be understood to be so by a skilled person. See for example Chothia et al., (1989) Conformations of immunoglobulin hypervariable regions; Nature 342, p 877-883.

As used herein, an “immunoassay” refers to any binding assay that uses an antibody capable of binding specifically to a target molecule to detect and quantify the target molecule.

The term “Complementarity Determining Region” or “CDR” are used to refer to hypervariable regions as defined by the Kabat system. See Kabat et al., Sequences of Proteins of Immunological Interest, 5th Ed. Public Health Service, National Institutes of Health, Bethesda, Md. (1991)

As use herein, the term “specifically binds” or is “specific for” refers to measurable and reproducible interactions such as binding between a target and an antibody, which is determinative of the presence of the target in the presence of a heterogeneous population of molecules including biological molecules. For example, an antibody that specifically binds a target (which can be an epitope) is an antibody that binds this target with greater affinity, avidity, more readily, and/or with greater duration than it binds other targets. In certain embodiments, an antibody that specifically binds a target has a dissociation constant (Kd) of ≤1 μM, ≤100 nM, ≤10 nM, ≤1 nM, or ≤0.1 nM. In certain embodiments, an antibody specifically binds an epitope on a protein that is conserved among the protein from different species. In another embodiment, specific binding can include, but does not require exclusive binding.

The term “specificity” refers to selective recognition of an antigen binding protein or antibody for a particular epitope of an antigen. Natural antibodies, for example, are monospecific. The term “multispecific” as used herein denotes that an antigen binding protein or an antibody has two or more antigen-binding sites of which at least two bind a different antigen or a different epitope of the same antigen. “Bispecific” as used herein denotes that an antigen binding protein or an antibody has two different antigen-binding specificities. The term “monospecific” antibody as used herein denotes an antibody that has one or more binding sites each of which bind the same epitope of the same antigen.

“Effector cells” are leukocytes which express one or more FcRs and perform effector functions. In one aspect, the effector cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils. The effector cells may be isolated from a native source, e.g., blood. Effector cells generally are lymphocytes associated with the effector phase, and function to produce cytokines (helper T cells), killing cells in infected with pathogens (cytotoxic T cells) or secreting antibodies (differentiated B cells).

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g., as described in Gazzano-Santoro et al., J. Immunol. Methods 202: 163 (1996), may be performed. Antibody variants with altered Fc region amino acid sequences and increased or decreased C1q binding capability are described in U.S. Pat. No. 6,194,551B1 and WO99/51642. The contents of those patent publications are specifically incorporated herein by reference. See, also, Idusogie et al. J. Immunol. 164: 4178-4184 (2000).

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity that reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present application. Specific illustrative and exemplary embodiments for measuring binding affinity are described in the following.

An “on-rate,” “rate of association,” “association rate,” or “km” as used herein can also be determined as described above using methods such as biolayer interferometry and surface plasmon resonance.

A “low-pH dissociation factor” as used herein is defined as the percentage of antibody dissociated at pH 5.8 from the antigen at 25° C., wherein the antibody is pre-bound to the antigen at pH 7.4. The low-pH dissociation factor may be measured by associating antibody and antigen (e.g. anti-C5 antibody and human C5) at pH 7.4 for 600 seconds, followed by a dissociation period of 600 seconds in a buffer at pH 5.8, and calculation of the percentage of antibody dissociated at pH 5.8 from the antigen. A “neutral-pH dissociation factor” is defined as the percentage of antibody dissociated at pH 7.4 from the antigen at 25° C., wherein the antibody is pre-bound to the antigen at pH 7.4. The neutral-pH dissociation factor may be measured by associating antibody and antigen (e.g. anti-C5 antibody and human C5) at pH 7.4 for 600 seconds, followed by a dissociation period of 600 seconds in a buffer at pH 7.4, and calculation of the percentage of antibody dissociated at pH 7.4 from the antigen. The antibody-antigen association and dissociation may be measured in various ways that are with the skill in the art, for instance, using biolayer interferometry.

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

“Isolated” means altered or removed from the natural state. For example, a nucleic acid or a peptide naturally present in its normal context in a living subject is not “isolated,” but the same nucleic acid or peptide partially or completely separated from the coexisting materials of its natural context is “isolated.” An isolated nucleic acid or protein can exist in substantially purified form, or can exist in a non-native environment such as, for example, a host cell.

The term “hybridoma,” as used herein refers to a cell resulting from the fusion of a B-lymphocyte and a fusion partner such as a myeloma cell. A hybridoma can be cloned and maintained indefinitely in cell culture and is able to produce monoclonal antibodies. A hybridoma can also be considered to be a hybrid cell.

The terms “nucleic acid molecule”, “nucleic acid” and “polynucleotide” may be used interchangeably, and refer to a polymer of nucleotides. Such polymers of nucleotides may contain natural and/or unnatural nucleotides, and include, but are not limited to, DNA, RNA, and PNA. “Nucleic acid sequence” refers to the linear sequence of nucleotides that comprise the nucleic acid molecule or polynucleotide. An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

“Complementary” as used herein to refer to a nucleic acid, refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, at least about 50%, and or at least about 75%, or at least about 90%, or at least about 95% of the nucleotide residues of the first portion are capable of base pairing with nucleotide residues in the second portion. In some embodiments, all nucleotide residues of the first portion are capable of base pairing with nucleotide residues in the second portion.

“Vector” as used herein may mean a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting there from. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The terms “polypeptide” and “peptide” are used interchangeably to refer to a polymer of amino acid residues, and are not limited to a minimum length. Such polymers of amino acid residues may contain natural or unnatural amino acid residues. Both full-length proteins and fragments thereof are encompassed by the definition. The terms also include post-expression modifications of the polypeptide, for example, glycosylation, sialylation, acetylation, phosphorylation, and the like. Furthermore, a “polypeptide” includes modifications, such as deletions, additions, and substitutions (generally conservative in nature), to the native sequence, as long as the polypeptide maintains the desired activity. These modifications may be deliberate, as through site-directed mutagenesis, or may be accidental, such as through mutations of hosts which produce the proteins or errors due to PCR amplification.

As used herein, “conjugated” refers to covalent attachment of one molecule to a second molecule.

“Variant” as the term is used herein, is a nucleic acid sequence or a peptide sequence that differs in sequence from a reference nucleic acid sequence or peptide sequence respectively, but retains essential biological properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis. In various embodiments, the variant sequence is at least 99%, at least 98%, at least 97%, at least 96%, at least 95%, at least 94%, at least 93%, at least 92%, at least 91%, at least 90%, at least 89%, at least 88%, at least 87%, at least 86%, at least 85% identical to the reference sequence.

The term “regulating” as used herein can mean any method of altering the level or activity of a substrate. Non-limiting examples of regulating with regard to a protein include affecting expression (including transcription and/or translation), affecting folding, affecting degradation or protein turnover, and affecting localization of a protein. Non-limiting examples of regulating with regard to an enzyme further include affecting the enzymatic activity. “Regulator” refers to a molecule whose activity includes affecting the level or activity of a substrate. A regulator can be direct or indirect. A regulator can function to activate or inhibit or otherwise modulate its substrate.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range. Reference to “about” a value or parameter herein includes (and describes) variations that are directed to that value or parameter per se. For example, description referring to “about X” includes description of “X”. As used herein, reference to “not” a value or parameter generally means and describes “other than” a value or parameter. For example, the method is not used to treat cancer of type X means the method is used to treat cancer of types other than X. The term “about X-Y” used herein has the same meaning as “about X to about Y.”

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed.

The “diluent” of interest herein is one which is pharmaceutically acceptable (safe and non-toxic for administration to a human) and is useful for the preparation of a liquid formulation, such as a formulation reconstituted after lyophilization. Exemplary diluents include sterile water, bacteriostatic water for injection (BWFI), a pH buffered solution (e.g. phosphate-buffered saline), sterile saline solution, Ringer's solution or dextrose solution. In an alternative embodiment, diluents can include aqueous solutions of salts and/or buffers.

A “preservative” is a compound which can be added to the formulations herein to reduce bacterial activity. The addition of a preservative may, for example, facilitate the production of a multi-use (multiple-dose) formulation. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol. The most preferred preservative herein is benzyl alcohol.

The terms “pharmaceutical formulation” and “pharmaceutical composition” refer to a preparation which is in such form as to permit the biological activity of the active ingredient(s) to be effective, and which contains no additional components which are unacceptably toxic to a subject to which the formulation would be administered. Such formulations may be sterile.

A “sterile” formulation is aseptic or essentially free from living microorganisms and their spores.

A “stable” formulation is one in which the protein therein essentially retains its physical and chemical stability and integrity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993). Stability can be measured at a selected temperature for a selected time period. For rapid screening, the formulation may be kept at 40° C. for 2 weeks to 1 month, at which time stability is measured. Where the formulation is to be stored at 2-8° C., generally the formulation should be stable at 30° C. or 40° C. for at least 1 month and/or stable at 2-8° C. for at least 2 years. Where the formulation is to be stored at 30° C., generally the formulation should be stable for at least 2 years at 30° C. and/or stable at 40° C. for at least 6 months. For example, the extent of aggregation during storage can be used as an indicator of protein stability. Thus, a “stable” formulation may be one wherein less than about 10% and preferably less than about 5% of the protein are present as an aggregate in the formulation. In other embodiments, any increase in aggregate formation during storage of the formulation can be determined.

A “reconstituted” formulation is one which has been prepared by dissolving a lyophilized protein or antibody formulation in a diluent such that the protein is dispersed throughout. The reconstituted formulation is suitable for administration (e.g. subcutaneous administration) to a patient to be treated with the protein of interest and, in certain embodiments, may be one which is suitable for parenteral or intravenous administration.

An “isotonic” formulation is one which has essentially the same osmotic pressure as human blood. Isotonic formulations will generally have an osmotic pressure from about 250 to 350 mOsm. The term “hypotonic” describes a formulation with an osmotic pressure below that of human blood. Correspondingly, the term “hypertonic” is used to describe a formulation with an osmotic pressure above that of human blood. Isotonicity can be measured using a vapor pressure or ice-freezing type osmometer, for example. The formulations of the present application can be hypertonic as a result of the addition of salt and/or buffer.

It is understood that embodiments described herein include “consisting” and/or “consisting essentially of” embodiments.

As used herein and in the appended claims, the singular forms “a,” “or,” and “the” include plural referents unless the context clearly dictates otherwise.

### II. pH-Dependent Anti-C5 Antibodies

The present application provides novel anti-human C5 antibodies and constructs having mutations in its VH and/or VL domains that render the antibodies pH sensitive in antigen binding. The anti-C5 antibodies harbor certain mutations, for example the Y32H mutation in the VH domain and bind more strongly to C5 at a neutral pH (e.g., about pH 7.4; such as that found in the blood) than it does at a more acidic pH (e.g., about pH 5.8; such as that found in the endosome).

Such pH-dependent binding provides for greater persistence of administered antibody or antibody fusion protein molecules, because immune complexes (i.e., anti-C5 antibody or a fragment thereof bound to C5) taken up by cells will dissociate in the acidic environment of the endosome and allow the freed antibody or antibody fusion protein to be recycled back out of the cell through the neonatal Fc receptor (FcRn) where it is available to bind to a new C5 molecule.

The pH dependence of an anti-C5 antibody can be assessed based on the dissociation of a C5-bound antibody at an acidic pH (e.g., pH 5.8) or at a neutral pH (e.g., pH 7.4). Low-pH dissociation factor, namely, the percentage of antibody dissociated at pH 5.8 from the antigen at 25° C., wherein the antibody is pre-bound to the antigen at pH 7.4, can be used to determine the dissociation of a C5-bound antibody at an acidic pH. The low-pH dissociation factor may be measured by associating antibody and antigen (e.g. anti-C5 antibody and human C5) at pH 7.4 for 600 seconds, followed by a dissociation period of 600 seconds in a buffer at pH 5.8, and calculation of the percentage of antibody dissociated at pH 5.8 from the antigen. In some embodiments, the low-pH dissociation factor of the anti-C5 antibody of the present invention is in the range of any one of about 5% to about 95%, about 10% to about 90%, about 15% to about 85%, about 20% to about 80%, about 20% to about 75%, about 20% to about 70%, about 20% to about 65%, about 20% to about 60%, about 25% to about 75%, about 25% to about 70%, about 25% to about 65%, about 25% to about 60%, about 30% to about 75%, about 30% to about 70%, about 30% to about 65%, about 30% to about 60%, about 35% to about 75%, about 35% to about 70%, about 35% to about 65%, about 35% to about 60%, about 40% to about 75%, about 40% to about 70%, about 40% to about 65%, about 40% to about 60%. In some embodiments, the low-pH dissociation factor of the anti-C5 antibody is no less than about any of 95%, 90%, 85%, 80%, 75%, 70%, 65%, 60%, 55%, 50%, 45%, 40%, 35%, 30%, 25%, 20%, 15%, 10%, or 5%.

Neutral-pH dissociation factor, namely, the percentage of antibody dissociated at pH 7.4 from the antigen at 25° C., wherein the antibody is pre-bound to the antigen at pH 7.4 and can be used to determine the dissociation of a C5-bound antibody at a neutral pH. The neutral-pH dissociation factor may be measured by associating antibody and antigen (e.g. anti-C5 antibody and human C5) at pH 7.4 for 600 seconds, followed by a dissociation period of 600 seconds in a buffer at pH 7.4, and calculation of the percentage of antibody dissociated at pH 7.4 from the antigen. In some embodiments, the neutral-pH dissociation factor of the anti-C5 antibody of the present invention is no more than about any of 20%, 18%, 16%, 14%, 12%, 10%, 9%, 8%, 7%, 6%, 5%, 4%, 3%, 2%, or 1%.

In some embodiments, the ratio of the low-pH dissociation factor over the neutral-pH dissociation factor of the anti-C5 antibody of the present invention is any one of 1 or more, 1.5 or more, 2 or more, 2.5 or more, 3 or more, 3.5 or more, 4 or more, 4.5 or more, 5 or more, 6 or more, 7 or more, 8 or more, 9 or more, 10 or more. In some embodiments, the percentage of dissociation of the antibody for C5 at pH 5.8 over the percentage of dissociation of the antibody for C5 at pH 7.4 is 4 or more.

In some embodiments, the anti-C5 antibody cross-reacts with cynomolgus (“cyno”) monkey C5.

The anti-C5 antibodies of the present application comprise at least one antigen binding portion comprising a heavy chain variable domain (VH) and a light chain variable domain (VL). Exemplary antigen binding fragments contemplated herein include, but are not limited to, Fab, Fab′, F(ab′)₂, and Fv fragments; diabodies; linear antibodies; single-chain antibody molecules (such as scFv); and multispecific antibodies formed from antibody fragments. Such antigen binding portion can be a full-length conventional antibody consisting of two heavy chains and two light chains, or an antigen binding fragment derived therefrom. In some embodiments, the VH comprises a Y32H mutation in reference to SEQ ID NO:1 under the AbM numbering system.

In some embodiments, the anti-C5 antibody comprises a Q38R mutation in the light chain, wherein the mutation in reference to SEQ ID NO:2 under the AbM numbering system. In some embodiments, the anti-C5 antibody comprises a VH comprising a Y32H mutation in reference to SEQ ID NO:1 and a VL comprising a Q38R mutation.

In some embodiment, the anti-C5 antibody further comprises an additional mutation (such as histidine mutations) in the variable domain (VL) of light chain. For example, in some embodiments, the anti-C5 antibody comprises a mutation in VL selected from the group consisting of E27H, E27F, E27L, E27M, E27W, E27I, N28H, N28F, N28L, N28M, N28W, Y30H, N28I, T52H, T52F, T52L, T52M, T52W, T52I, L54H, L54F, L54N, L54M, L54W, L54I, D56H, D56F, D56L, D56M, D56W, D56I, T58H, T58F, T58L, T58M, T58W, T58I, T97H, T97F, T97L, T97M, T97W, T97I, wherein the mutation in reference to SEQ ID NO:2 under the AbM numbering system. In some embodiments, the mutation is a histidine mutation, such as a histidine mutation at any one of the following positions in reference to SEQ ID NO:2:

DIQMTQSPSSLSASVGDRVTITCGASENIYGALNWYQQKPGKAPKLLIY
GATNLADGVPSRFSGSGSGTDFTLTISSLQPEDFATYYCQNVLNTPLTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

In some embodiments, the anti-C5 antibody comprises a mutation in VL selected from the group consisting of E27H, N28H, Y30H, T52H, L54H, D56H, T58H, T97H, wherein the mutation is in reference to SEQ ID NO:2 under the AbM numbering system.

Thus, in some embodiments, there is provided an anti-C5 antibody comprising a VH and a VL, wherein the VH comprises a Y32H mutation, wherein the mutation is in reference to SEQ ID NO:1 under the AbM system. In some embodiments, there is provided an anti-C5 antibody comprising a VH and VL, wherein the VH comprises a Y32H mutation, and wherein the VL comprise a Q38 mutation, wherein the mutation is in reference to SEQ ID NO:2 under the AbM numbering system. In some embodiments, there is provided an anti-C5 antibody comprising a VH and VL, wherein the VH comprises a Y32H mutation, and wherein the VL comprise a Q38 mutation and a histidine substitution, wherein the mutation is in reference to SEQ ID NO:2 under the AbM numbering system. In some embodiments, there is provided an anti-C5 antibody comprising a VH and VL, wherein the VH comprises a Y32H mutation, and wherein the VL comprise a Q38 mutation and at least one mutation selected from the group consisting of E27H, E27F, E27L, E27M, E27W, E27I, N28H, N28F, N28L, N28M, N28W, N28I, Y30H, T52H, T52F, T52L, T52M, T52W, T52I, L54H, L54F, L54N, L54M, L54W, L54I, D56H, D56F, D56L, D56M, D56W, D56I, T58H, T58F, T58L, T58M, T58W, T58I, T97H, T97F, T97L, T97M, T97W, T97I, wherein the mutation is in reference to SEQ ID NO:2 under the AbM numbering system. In some embodiments, there is provided an anti-C5 antibody comprising a VH and VL, wherein the VH comprises a Y32H mutation, and wherein the VL comprise a Q38 mutation and at least one mutation selected from the group consisting of E27H, N28H, Y30H, T52H, L54H, D56H, T58H, T97H, wherein the mutation is in reference to SEQ ID NO:2 under the AbM numbering system. In some embodiments, the anti-C5 antibody further comprises an Fc region (such as the Fc region of an IgG4, for example an Fc region comprises PLA mutations: S228P, M428L, and N434A). In some embodiments, the anti-C5 antibody cross-reacts with cyno monkey C5. In some embodiments, the anti-C5 antibody binds more strongly at a neutral pH (such as pH 7.4) than it does at an acidic pH (such as pH 5.8). In some embodiments, the low-pH dissociation factor of the anti-C5 antibody is no less than about any of 95%, 90%, 85%, 80%, 75%, 70%, 65%, 60%, 55%, 50%, 45%, 40%, 35%, 30%, 25%, 20%, 15%, 10%, or 5%. In some embodiments, the neutral-pH dissociation factor of the anti-C5 antibody is no more than about any of 20%, 18%, 16%, 14%, 12%, 10%, 9%, 8%, 7%, 6%, 5%, 4%, 3%, 2%, or 1%. In some embodiments, the ratio of the low-pH dissociation factor over the neutral-pH dissociation factor of the anti-C5 antibody is about any of 1.5, 2, 2.5, 3, 3.5, 4, 4.5, 5, 5.5, 6, 6.5, 7, 7.5, 8, 8.5, 9, 9.5, or 10. In some embodiments, the ratio of the low-pH dissociation factor over the neutral-pH dissociation factor of the anti-C5 antibody is no less than about any of 1.5, 2, 2.5, 3, 3.5, 4, 4.5, 5, 5.5, 6, 6.5, 7, 7.5, 8, 8.5, 9, 9.5, or 10. In some embodiments, the ratio of the low-pH dissociation factor over the neutral-pH dissociation factor of the anti-C5 antibody is no less than 4.

In some embodiments, the anti-C5 antibody (for example any one of the Y32H-containing anti-C5 antibody described herein) comprises a heavy chain CDR1 (“H-CDR1”) comprising the amino acid sequence of SEQ ID NO:3 or a variant thereof comprising one, two, or three amino acid substitutions; a heavy chain CDR2 (“H-CDR2”) comprising the amino acid sequence of SEQ ID NO:4 or a variant thereof comprising one, two, or three amino acid substitutions; a heavy chain CDR3 (“H-CDR3”) comprising the amino acid sequence of SEQ ID NO:5 or a variant thereof comprising one, two, or three amino acid substitutions; a light chain CDR1 (“L-CDR1”) comprising the amino acid sequence of SEQ ID NO:6 or a variant thereof comprising one, two, or three amino acid substitutions; a light chain CDR2 (“L-CDR2”) comprising the amino acid sequence of SEQ ID NO:7 or a variant thereof comprising one, two, or three amino acid substitutions; and a light chain CDR3 (“L-CDR3”) comprising the amino acid sequence of SEQ ID NO:8 or a variant thereof comprising one, two, or three amino acid substitutions. In some embodiments, the anti-C5 antibody comprises a heavy chain CDR1 (“H-CDR1”) comprising the amino acid sequence of SEQ ID NO:3; a heavy chain CDR2 (“H-CDR2”) comprising the amino acid sequence of SEQ ID NO:4; a heavy chain CDR3 (“H-CDR3”) comprising the amino acid sequence of SEQ ID NO:5; a light chain CDR1 (“L-CDR1”) comprising the amino acid sequence of SEQ ID NO:6 or a variant thereof comprising one, two, or three amino acid substitutions; a light chain CDR2 (“L-CDR2”) comprising the amino acid sequence of SEQ ID NO:7 or a variant thereof comprising one, two, or three amino acid substitutions; and a light chain CDR3 (“L-CDR3”) comprising the amino acid sequence of SEQ ID NO:8 or a variant thereof comprising one, two, or three amino acid substitutions.

In some embodiments (independent of or in addition to the CDR sequences described here), the anti-C5 antibody comprises a VH comprising the amino acid sequence SEQ ID NO:9 or a variant thereof that is at least about any one of 50%, 60%, 70%, 80%, 85%, 90%, 95% or more of amino acid homology to SEQ ID NO:9; and a VL comprising the amino acid sequence of SEQ ID NO:2 or a variant thereof that is at least about any one of 60%, 70%, 80%, 85%, 90%, 95%, 96%, 97%, 98%, 99% or more of amino acid homology to SEQ ID NO:2. In some embodiments, the anti-C5 antibody comprises a VH comprising the amino acid sequence SEQ ID NO:9 and a VL comprising the amino acid sequence of SEQ ID NO:2 or a variant thereof that is at least about any one of 60%, 70%, 80%, 85%, 90%, 95%, 96%, 97%, 98%, 99% or more of amino acid homology to SEQ ID NO:2. In some embodiments, the anti-C5 antibody further comprises an Fc region (such as the Fc region of an IgG4, for example an Fc region comprises PLA mutation: S228P, M428L, and N434A). In some embodiments, the anti-C5 antibody cross-reacts with cyno monkey C5. In some embodiments, the anti-C5 antibody binds more strongly at a neutral pH (such as pH 7.4) than it does at an acidic pH (such as pH 5.8). In some embodiments, the low-pH dissociation factor of the anti-C5 antibody is no less than about any of 95%, 90%, 85%, 80%, 75%, 70%, 65%, 60%, 55%, 50%, 45%, 40%, 35%, 30%, 25%, 20%, 15%, 10%, or 5%. In some embodiments, the neutral-pH dissociation factor of the anti-C5 antibody is no more than about any of 20%, 18%, 16%, 14%, 12%, 10%, 9%, 8%, 7%, 6%, 5%, 4%, 3%, 2%, or 1%. In some embodiments, the ratio of the low-pH dissociation factor over the neutral-pH dissociation factor of the anti-C5 antibody is about any of 1.5, 2, 2.5, 3, 3.5, 4, 4.5, 5, 5.5, 6, 6.5, 7, 7.5, 8, 8.5, 9, 9.5, or 10. In some embodiments, anti-C5 antibody comprises an H-CDR1 comprising the amino acid sequence of SEQ ID NO:3, an H-CDR2 comprising the amino acid sequence of SEQ ID NO:4, an H-CDR3 comprising the amino acid sequence of SEQ ID NO:5, an L-CDR1 comprising the amino acid sequence of SEQ ID NO:6, an L-CDR2 comprising the amino acid sequence of SEQ ID NO:7, and an L-CDR3 comprising the amino acid sequence of SEQ ID NO:8. In some embodiments, the anti-C5 antibody comprises a VH comprising the amino acid sequence of SEQ ID NO:9; and a VL comprising the amino acid sequence of SEQ ID NO:2. In some embodiments, the anti-C5 antibody comprises a VH comprising the amino acid sequence of SEQ ID NO:9; and a VL comprising the amino acid sequence of SEQ ID NO:18. In some embodiments, the anti-C5 antibody further comprises an Fc region (such as the Fc region of an IgG4, for example an Fc region comprises PLA mutation: S228P, M428L, and N434A). In some embodiment, the Fc region comprises S228P, M428L, and N434A, wherein the mutations are relative to SEQ ID NO:26 under the EU numbering system. In some embodiments, the Fc region comprises amino acid sequence of SEQ ID NO:27.

In some embodiments, the anti-C5 antibody comprises an H-CDR1 comprising the amino acid sequence of SEQ ID NO:3; an H-CDR2 comprising the amino acid sequence of SEQ ID NO:4; an H-CDR3 comprising the amino acid sequence of SEQ ID NO:5; an L-CDR1 comprising the amino acid sequence of SEQ ID NO:11; an L-CDR2 comprising the amino acid sequence of SEQ ID NO:7; and an L-CDR3 comprising the amino acid sequence of SEQ ID NO:8. In some embodiments, the anti-C5 antibody comprises a VH comprising the amino acid sequence of SEQ ID NO:9; and a VL comprising the amino acid sequence of SEQ ID NO:19. In some embodiments, the anti-C5 antibody further comprises an Fc region (such as the Fc region of an IgG4, for example an Fc region comprises PLA mutation: S228P, M428L, and N434A). In some embodiment, the Fc region comprises S228P, M428L, and N434A, wherein the mutations are relative to SEQ ID NO:26 under the EU numbering system. In some embodiments, the Fc region comprises amino acid sequence of SEQ ID NO:27.

In some embodiments, the anti-C5 antibody comprises an H-CDR1 comprising the amino acid sequence of SEQ ID NO:3; an H-CDR2 comprising the amino acid sequence of SEQ ID NO:4; an H-CDR3 comprising the amino acid sequence of SEQ ID NO:5; an L-CDR1 comprising the amino acid sequence of SEQ ID NO:12; an L-CDR2 comprising the amino acid sequence of SEQ ID NO:7; and an L-CDR3 comprising the amino acid sequence of SEQ ID NO:8. In some embodiments, the anti-C5 antibody comprises a VH comprising the amino acid sequence of SEQ ID NO:9; and a VL comprising the amino acid sequence of SEQ ID NO:20. In some embodiments, the anti-C5 antibody further comprises an Fc region (such as the Fc region of an IgG4, for example an Fc region comprises PLA mutation: S228P, M428L, and N434A). In some embodiment, the Fc region comprises S228P, M428L, and N434A, wherein the mutations are relative to SEQ ID NO:26 under the EU numbering system. In some embodiments, the Fc region comprises amino acid sequence of SEQ ID NO:27.

In some embodiments, the anti-C5 antibody comprises an H-CDR1 comprising the amino acid sequence of SEQ ID NO:3; an H-CDR2 comprising the amino acid sequence of SEQ ID NO:4; an H-CDR3 comprising the amino acid sequence of SEQ ID NO:5; an L-CDR1 comprising the amino acid sequence of SEQ ID NO:13; an L-CDR2 comprising the amino acid sequence of SEQ ID NO:7; and an L-CDR3 comprising the amino acid sequence of SEQ ID NO:8. In some embodiments, the anti-C5 antibody comprises a VH comprising the amino acid sequence of SEQ ID NO:9; and a VL comprising the amino acid sequence of SEQ ID NO:21. In some embodiments, the anti-C5 antibody further comprises an Fc region (such as the Fc region of an IgG4, for example an Fc region comprises PLA mutation: S228P, M428L, and N434A). In some embodiment, the Fc region comprises S228P, M428L, and N434A, wherein the mutations are relative to SEQ ID NO:26 under the EU numbering system. In some embodiments, the Fc region comprises amino acid sequence of SEQ ID NO:27.

In some embodiments, the anti-C5 antibody comprises an H-CDR1 comprising the amino acid sequence of SEQ ID NO:3; an H-CDR2 comprising the amino acid sequence of SEQ ID NO:4; an H-CDR3 comprising the amino acid sequence of SEQ ID NO:5; an L-CDR1 comprising the amino acid sequence of SEQ ID NO:6; an L-CDR2 comprising the amino acid sequence of SEQ ID NO:14; and an L-CDR3 comprising the amino acid sequence of SEQ ID NO:8. In some embodiments, the anti-C5 antibody comprises a VH comprising the amino acid sequence of SEQ ID NO:9; and a VL comprising the amino acid sequence of SEQ ID NO:22. In some embodiments, the anti-C5 antibody further comprises an Fc region (such as the Fc region of an IgG4, for example an Fc region comprises PLA mutation: S228P, M428L, and N434A). In some embodiment, the Fc region comprises S228P, M428L, and N434A, wherein the mutations are relative to SEQ ID NO:26 under the EU numbering system. In some embodiments, the Fc region comprises amino acid sequence of SEQ ID NO:27.

In some embodiments, the anti-C5 antibody comprises an H-CDR1 comprising the amino acid sequence of SEQ ID NO:3; an H-CDR2 comprising the amino acid sequence of SEQ ID NO:4; an H-CDR3 comprising the amino acid sequence of SEQ ID NO:5; an L-CDR1 comprising the amino acid sequence of SEQ ID NO:6; an L-CDR2 comprising the amino acid sequence of SEQ ID NO:15; and an L-CDR3 comprising the amino acid sequence of SEQ ID NO:8. In some embodiments, the anti-C5 antibody comprises a VH comprising the amino acid sequence of SEQ ID NO:9; and a VL comprising the amino acid sequence of SEQ ID NO:23. In some embodiments, the anti-C5 antibody further comprises an Fc region (such as the Fc region of an IgG4, for example an Fc region comprises PLA mutation: S228P, M428L, and N434A). In some embodiment, the Fc region comprises S228P, M428L, and N434A, wherein the mutations are relative to SEQ ID NO:26 under the EU numbering system. In some embodiments, the Fc region comprises amino acid sequence of SEQ ID NO:27.

In some embodiments, the anti-C5 antibody comprises an H-CDR1 comprising the amino acid sequence of SEQ ID NO:3; an H-CDR2 comprising the amino acid sequence of SEQ ID NO:4; an H-CDR3 comprising the amino acid sequence of SEQ ID NO:5; an L-CDR1 comprising the amino acid sequence of SEQ ID NO:6; an L-CDR2 comprising the amino acid sequence of SEQ ID NO:16; and an L-CDR3 comprising the amino acid sequence of SEQ ID NO:8. In some embodiments, the anti-C5 antibody comprises a VH comprising the amino acid sequence of SEQ ID NO:9; and a VL comprising the amino acid sequence of SEQ ID NO:24. In some embodiments, the anti-C5 antibody further comprises an Fc region (such as the Fc region of an IgG4, for example an Fc region comprises PLA mutation: S228P, M428L, and N434A). In some embodiment, the Fc region comprises S228P, M428L, and N434A, wherein the mutations are relative to SEQ ID NO:26 under the EU numbering system. In some embodiments, the Fc region comprises amino acid sequence of SEQ ID NO:27.

In some embodiments, the anti-C5 antibody comprises an H-CDR1 comprising the amino acid sequence of SEQ ID NO:3; an H-CDR2 comprising the amino acid sequence of SEQ ID NO:4; an H-CDR3 comprising the amino acid sequence of SEQ ID NO:5; an L-CDR1 comprising the amino acid sequence of SEQ ID NO:6; an L-CDR2 comprising the amino acid sequence of SEQ ID NO:7; and an L-CDR3 comprising the amino acid sequence of SEQ ID NO:17. In some embodiments, the anti-C5 antibody comprises a VH comprising the amino acid sequence of SEQ ID NO:9; and a VL comprising the amino acid sequence of SEQ ID NO:25. In some embodiments, the anti-C5 antibody further comprises an Fc region (such as the Fc region of an IgG4, for example an Fc region comprises PLA mutation: S228P, M428L, and N434A). In some embodiment, the Fc region comprises S228P, M428L, and N434A, wherein the mutations are relative to SEQ ID NO:26 under the EU numbering system. In some embodiments, the Fc region comprises amino acid sequence of SEQ ID NO:27.

In some embodiments, the anti-C5 antibody comprises an Fc region, such as a human Fc region. In some embodiments, the Fc region is derived from an IgG molecule, such as any one of the IgG1, IgG2, IgG3, or IgG4 subclass. In some embodiments, the Fc region is capable of mediating an antibody effector function, such as ADCC (antibody-dependent cell-mediated cytotoxicity) and/or CDC (complement-dependent cytotoxicity). For example, antibodies of subclass IgG1, IgG2, and IgG3 with wild-type Fc sequences usually show complement activation including CIq and C3 binding, whereas IgG4 does not activate the complement system and does not bind CIq and/or C3. In some embodiments, the Fc region comprises a modification that reduces binding affinity of the Fc region to an Fc receptor. In some embodiments, the Fc region is an IgG1 Fc. In some embodiments, the IgG1 Fc comprises one or mutations in positions 233-236, such as L234A and/or L235A. In some embodiments, the Fc region is an IgG4 Fc. In some embodiments, the IgG4 Fc region comprises the amino acid sequence of SEQ ID NO: 26. In some embodiments, the IgG4 Fc comprises mutations. See, for example, Armour K L et al., Eur J. Immunol. 1999; 29: 2613; and Shields R L et al., J. Biol. Chem. 2001; 276: 6591. In some embodiments, the Fc region comprises one or more mutations selected from the group consisting of S228P, M428L, and N434A, wherein the mutations are relative to SEQ ID NO:26 under the EU numbering system. In some embodiments, the Fc region comprises mutations S228P, M428L, and N434A. In some embodiments, the Fc region comprises amino acid sequence of SEQ ID NO:27.

In some embodiments, the anti-C5 antibody may further comprise signal peptides. In some embodiments, the heavy chain of the anti-C5 antibody may comprise a signal peptide comprising the amino acid sequence of SEQ ID NO:31. In some embodiments, the light chain of the anti-C5 antibody may comprise a signal peptide comprising the amino acid sequence of SEQ ID NO:32.

### C5 Proteins and C5 Binding Analysis

The complement system plays an important role in the pathology of many autoimmune, inflammatory and ischemic diseases. Inappropriate complement activation and its deposition on host cells can lead to complement-mediated lysis and/or injury of cells and target tissues, as well as tissue destruction due to the generation of powerful mediators of inflammation. The complement system, also known as complement cascade, is a part of the immune system that enhances (complements) the ability of antibodies and phagocytic cells to clear microbes and damaged cells from an organism, promote inflammation, and attack the pathogen's cell membrane. It is part of the innate immune system, which is not adaptable and does not change during an individual's lifetime. The complement system can, however, be recruited and brought into action by antibodies generated by the adaptive immune system.

Without being bound by any theory or hypothesis, there are three known complement pathways: the alternative complement pathway (AP), the classical pathway (CP), and the lectin pathway (LP). Generally, the CP is initiated by antigen-antibody complexes, the LP is activated by binding of lectins to sugar molecules on microbial surfaces, while the AP is constitutively active at a low level but can be quickly amplified on bacterial, viral, and parasitic cell surfaces due to the lack of regulatory proteins. Host cells are usually protected from AP complement activation by regulatory proteins. But in some situations, such as when the regulatory proteins are defective or missing, the AP can also be activated uncontrollably on host cells, leading to complement-mediated disease or disorder. The CP consists of components C1, C2, C4 and converges with the AP at the C3 activation step. The LP consists of mannose-binding lectins (MBLs) and MBL-associated serine proteases (MASPs) and shares with the CP the components C4 and C2. The AP consists of components C3 and several factors, such as factor B, factor D, properdin, and the fluid phase regulator factor H. Complement activation consists of three stages: (a) recognition, (b) enzymatic activation, and (c) membrane attack leading to cell death. The first phase of CP complement activation begins with C1. C1 is made up of three distinct proteins: a recognition subunit, C1q, and the serine protease subcomponents, C1r and C1s, which are bound together in a calcium-dependent tetrameric complex, C1r2 s2. An intact C1 complex is necessary for physiological activation of C1 to result. Activation occurs when the intact C1 complex binds to immunoglobulin complexed with antigen. This binding activates C1s which then cleaves both the C4 and C2 proteins to generate C4a and C4b, as well as C2a and C2b. The C4b and C2a fragments combine to form the C3 convertase, C4b2a, which in turn cleaves C3 to form C3a and C3b. Activation of the LP is initiated by MBL binding to certain sugars on the target surface and this triggers the activation of MBL-associated serine proteases (MASPs) which then cleave C4 and C2 in a manner analogous to the activity of C1s of the CP, resulting in the generation of the C3 convertase, C4b2a. Thus, the CP and LP are activated by different mechanisms but they share the same components C4 and C2 and both pathways lead to the generation of the same C3 convertase, C4b2a. The cleavage of C3 by C4b2a into C3b and C3a is a central event of the complement pathway for two reasons. It initiates the AP amplification loop because surface deposited C3b is a central intermediate of the AP C3 convertase C3bBb. Both C3a and C3b are biologically important. C3a is proinflammatory and together with C5a are referred to as anaphylatoxins. C3b and its further cleavage products also bind to complement receptors present on neutrophils, eosinophils, monocytes and macrophages, thereby facilitating phagocytosis and clearance of C3b-opsonized particles. Finally, C3b can associate with C4b2a or C3bBb to form the C5 convertase of the CP and LP, and AP, respectively, to activate the terminal complement sequence, leading to the production of C5a, a potent proinflammatory mediator, and the assembly of the lytic membrane attack complex (MAC), C5-C9.

Defective complement action is a cause of several human glomerular diseases including atypical hemolytic uremic syndrome (aHUS), anti-neutrophil cytoplasmic antibody mediated vasculitis (ANCA), C3 glomerulopathy, IgA nephropathy, immune complex membranoproliferative glomerulonephritis, renal ischemic reperfusion injury, lupus nephritis, membranous nephropathy, and chronic transplant mediated glomerulopathy. Aberrant complement component activation has been proposed as markers in various types of cancers and their clinical outcomes. Lung cancer patients show significantly higher plasma levels of complement proteins and activation fragments than do control donors, and elevated complement levels are correlated with lung tumor size. Complement-related proteins are also elevated in biological fluids from patients with other types of tumor. See, for example, Pio et al. Semin Immunol. 2013 February; 25(1): 54-64. Inhibition of the complement cascade has been proposed for glomerular diseases and cancer treatment. The complement inhibitors Eculizumab (Soliris), Berinert, or Cinryze are currently approved by the Food and Drug administration (FDA) in the US and the European Medicines Agency (EMA).

C5 is a 190 kDa protein (SEQ ID NO:30) found in normal serum at approximately 80 micro g/ml (0.4 micro M). C5 is glycosylated with about 1.5-3% of its mass attributed to carbohydrate. Mature C5 is a heterodimer of 115 kDa alpha chain that is disulfide linked to 75 kDa beta chain. C5 is synthesized as a single chain precursor protein (pro-C5 precursor) of 1676 amino acids (see, e.g., PTL1 and PTL2). The pro-C5 precursor is cleaved to yield the beta chain as an amino terminal fragment and the alpha chain as a carboxyl terminal fragment. The alpha chain and the beta chain polypeptide fragments are connected to each other via a disulfide bond and constitute the mature C5 protein. Mature C5 is cleaved into the C5a and C5b fragments during activation of the complement pathways. C5a is cleaved from the alpha chain of C5 by C5 convertase as an amino terminal fragment comprising the first 74 amino acids of the alpha chain. The remaining portion of mature C5 is fragment C5b, which contains the rest of the alpha chain disulfide bonded to the beta chain. Approximately 20% of the 11 kDa mass of C5a is attributed to carbohydrate.

In some embodiments, the activity of the complement pathway that is inhibited using the anti-C5 antibody is a complement pathway activation induced by at least one of the group selected from a lipopolysaccharide (LPS), lipooligosaccharide (LOS), pathogen-associated molecular patterns (PAMPs) and danger-associated molecular patterns (DAMPs). In another embodiment, the activity of complement signaling that is inhibited using a method of invention is the generation of C5a protein. In another embodiment, the activity of complement signaling that is inhibited using a method of invention is the generation of C5b protein.

In some embodiments, the anti-C5 antibody is further conjugated to a protein, a peptide or another compound. In some embodiments, the protein, peptide or other compound to which the anti-C5 antibody is conjugated is a targeting moiety (i.e., the targeting moiety specifically binds to a molecule other than human-C5). In some embodiments, the protein, peptide, or other compound to which the human-C5 binding antibody, fusion protein, or antibody fragment thereof is conjugated to is an effector molecule (e.g., a cytotoxic molecule).

In some embodiments, the activity of complement signaling that is inhibited using a method of invention is the generation of C5a protein, C5b protein, C3a protein, C3b protein, or any combination thereof. In some embodiments, the activity of complement signaling that is inhibited using a method of the invention is the formation of MAC.

In some embodiments, binding of the anti-C5 antibody or fusion protein to human-C5 is associated with a reduction in the generation of C5a or C5b and the formation of MAC in the complement activation pathway in an intact organism. In some embodiments, the anti-C5 antibody or fusion protein is further capable of inhibiting the activation of human C3. In some embodiments, the invention is a protein or a polypeptide capable of binding to and inhibiting the activation of human C5. In some embodiments, the anti-C5 antibody fusion protein is capable of binding to and/or inhibiting the activation of human C3, human C5, or both. In some embodiments, the anti-C5 antibody is associated with a reduction in the generation of C5a or C5b and the formation of MAC in an intact organism.

Binding affinity and specificity of the anti-C5 antibody described herein can be determined experimentally by methods known in the art. For example, the binding of an antibody to a protein antigen can be detected and/or quantified using a variety of techniques such as, but not limited to, Western blot, dot blot, surface plasmon resonance (SPR) method (e.g., BIAcore system; Pharmacia Biosensor AB, Uppsala, Sweden and Piscataway, N.J.), Bio-Layer Interferometry (BLI) (e.g. Octet system, ForteBio), RIA, ECL, IRMA, EIA, peptide scans, and enzyme-linked immunosorbent assay (ELISA). See, e.g., Benny K. C. Lo (2004) “Antibody Engineering: Methods and Protocols.” Humana Press (ISBN: 1588290921); Borrebaek (1992) “Antibody Engineering, A Practical Guide.” W.H. Freeman and Co., N.Y.; Borrebaek (1995) “Antibody Engineering.” 2nd Edition, Oxford University Press, N.Y., Oxford; Johne et al. (1993). J Immunol Meth 160:191-198; Jonsson et al. (1993) Ann Biol Clin 51:19-26; and Jonsson et al. (1991) Biotechniques 11:620-627. In addition, methods for measuring the affinity (e.g., dissociation and association constants by BLI) are set forth in the working examples.

Methods for determining whether a particular antibody described herein inhibits C5 cleavage are known in the art. Inhibition of human complement component C5 can reduce the cell-lysing ability of complement in a subject's body fluids. Such reductions of the cell-lysing ability of complement present in the body fluid(s) can be measured by methods well known in the art such as, for example, by a conventional hemolytic assay such as the hemolysis assay in chicken erythrocyte hemolysis method as described in, e.g., Hillmen et al. (2004) N Engl. J Med 350(6):552. Methods for determining whether a candidate compound inhibits the cleavage of human C5 into forms C5a and C5b are known in the art and described in, e.g., Thomas et al. (1996) Mol Immunol 33(17-18): 1389-401; and Evans et al. (1995) Mol Immunol 32(16): 1183-95. For example, the concentration and/or physiologic activity of C5a and C5b in a body fluid can be measured by methods well known in the art. Methods for measuring C5a concentration or activity include, e.g., chemotaxis assays, RIAs, or ELISAs (see, e.g., Wurzner et al. (1991) Complement Inflamm 8:328-340). For C5b, hemolytic assays or assays for soluble C5b-9 as discussed herein can be used. Other assays known in the art can also be used. Using assays of these or other suitable types, candidate agents capable of inhibiting human complement component C5 can be screened.

Hemolytic assays can be used to determine the inhibitory activity of an anti-C5 antibody on complement activation. In order to determine the effect of an anti-C5 antibody on classical complement pathway-mediated hemolysis in a serum test solution in vitro, for example, sheep erythrocytes coated with hemolysin or chicken erythrocytes sensitized with anti-chicken erythrocyte antibody are used as target cells. The percentage of lysis is normalized by considering 100% lysis equal to the lysis occurring in the absence of the inhibitor. To determine the effect of anti-C5 antibody on alternative pathway-mediated hemolysis, unsensitized rabbit or guinea pig erythrocytes are used as the target cells. The percentage of lysis is normalized by considering 100% lysis equal to the lysis occurring in the absence of the inhibitor.

### Anti-C5 Antibody Fusion Protein

The anti-C5 antibodies described herein can be conjugated (e.g., fused) to another moiety. In some embodiments, the other moiety is a targeting moiety (such as an antigen binding polypeptide). In some embodiments, the other moiety is an effector moiety (e.g., a drug, a toxin, etc.). In some embodiments, the other moiety is a complement associated protein or functional fragment thereof.

The complement system is tightly regulated by a network of proteins known as regulators of complement activation that help distinguish target cells as self or non-self. A subset of this family of proteins, complement control proteins (also known as sushi domains) are characterized by domains of conserved repeats that direct interaction with components of the complement system. Most complement control proteins prevent activation of the complement system on the surface of host cells and protect host tissues against damage caused by autoimmunity. Because of this, these proteins play important roles in autoimmune disorders and cancers. Exemplary complement control proteins include, but are not limited to, membrane cofactor protein, MCP (CD46); decay accelerating factor, DAF (CD55); protectin (CD59); complement C3b/C4b receptor 1, CR1 (CD35); complement regulator of the immunoglobulin superfamily, CRIg; factor H; and C4-binding protein (C4 bp). The anti-C5 antibody described herein can be fused to any one of these complement associated proteins or fragments thereof.

In some embodiments, the anti-C5 antibody is fused to Factor H or a functional fragment thereof. Factor H (FH) is a single polypeptide chain plasma glycoprotein. The protein is composed of 20 conserved short consensus repeat (SCR) domains of approximately 60 amino acids, arranged in a continuous fashion like a string of beads, separated by short linker sequences of 2-6 amino acids each. Factor H binds to C3b, accelerates the decay of the alternative pathway C3-convertase (C3bBb), and acts as a cofactor for the proteolytic inactivation of C3b by factor I. Alternative pathway amplification is initiated when circulating factor B binds to activated C3b. This complex is then cleaved by circulating factor D to yield an enzymatically active C3 convertase complex, C3bBb. C3bBb cleaves additional C3 generating C3b, which drives inflammation and also further amplifies the activation process, generating a positive feedback loop. Factor H is a key regulator (inhibitor) of the alternative complement pathway activation and initiation mechanisms that competes with factor B for binding to conformationally altered C3 (hydrolyzed form of C3 or C3(H2O)) in the tick-over mechanism and to C3b in the amplification loop. Binding of C3b to Factor H also leads to degradation of C3b by factor I to the inactive form iC3b (also designated C3bi), thus exerting a further check on complement activation. Factor H regulates complement in the fluid phase, circulating at a plasma concentration of approximately 500 μg/ml, but its binding to cells is a regulated phenomenon enhanced by the presence of a negatively charged surface as well as fixed C3b, iC3b, C3dg or C3d. See, for example, Jozsi et al., Histopathol. (2004) 19:251-258.

In some embodiments, the complement associated protein described herein comprises at factor-H (FH) protein or a fragment thereof. In one embodiment, the fragment of factor H comprises at least one SCR domains of the factor H protein. In one embodiment, the fragment of factor H comprises at least two SCR domains of the factor H protein. In one embodiment, the fragment of factor H comprises at least three SCR domains of the factor H protein. In one embodiment, the fragment of factor H comprises at least four SCR domains of the factor H protein. In one embodiment, the fragment of factor H comprises at least five SCR domains of the factor H protein. In one embodiment, the fragment of factor H comprises at least six SCR domains of the factor H protein. In one embodiment, the fragment of factor H comprises at least seven SCR domains of the factor H protein. In one embodiment, the fragment of factor H comprises at least eight SCR domains of the factor H protein. In one embodiment, the fragment of factor H comprises at least nine SCR domains of the factor H protein. In one embodiment, the fragment of factor H comprises at least ten SCR domains of the factor H protein. In one embodiment, the fragment of factor H comprises at least eleven SCR domains of the factor H protein. In one embodiment, the fragment of factor H comprises at least twelve SCR domains of the factor H protein. In one embodiment, the fragment of factor H comprises at least thirteen SCR domains of the factor H protein. In one embodiment, the fragment of factor H comprises at least fourteen SCR domains of the factor H protein. In one embodiment, the fragment of factor H comprises at least fifteen SCR domains of the factor H protein. In one embodiment, the fragment of factor H comprises at least sixteen SCR domains of the factor H protein. In one embodiment, the fragment of factor H comprises at least seventeen SCR domains of the factor H protein. In one embodiment, the fragment of factor H comprises at least eighteen SCR domains of the factor H protein. In one embodiment, the fragment of factor H comprises at least nineteen SCR domains of the factor H protein. In one embodiment, the fragment of factor H comprises at least twenty SCR domains of the factor H protein.

In one embodiment, the fragment of factor H comprises SCR domains 1-20 of the factor H protein. In one embodiment, the fragment of factor H comprises SCR domains 1-5 of the factor H protein. In one embodiment, the fragment of factor H comprises SCR domains 1-4 of the factor H protein. In one embodiment, the fragment of factor H comprises SCR domains 2-5 of the factor H protein. In one embodiment, the fragment of factor H comprises SCR domains 3-5 of the factor H protein. In one embodiment, the fragment of factor H comprises SCR domains 4 and 5 of the factor H protein. In one embodiment, the fragment of factor H comprises SCR domain 5 of the factor H protein.

In some embodiments, there is provided a fusion protein comprising a fusion protein partner linked to any one of the anti-C5 antibodies with at least one linker. In one embodiment, the fusion protein comprises a fusion protein partner bound to any one of the anti-C5 antibodies described herein without a linker. In one embodiment, the fusion protein comprises a fusion protein partner bound to C-terminal of the heavy chain of any one of the anti-C5 antibodies described herein. In one embodiment, the fusion protein comprises a fusion protein partner bound to N-terminal of light or heavy chain of any one of the anti-C5 antibodies described herein.

In one embodiment, the anti-C5 antibody fusion protein further comprises a complement control protein or a fragment of a complement control protein. In one embodiment, the complement control protein or fragment of complement control protein is an inhibitor of C3 convertase. In one embodiment, the C3 convertase is the alternative pathway C3 convertase C3bBb. In one embodiment, the C3 convertase is the classical pathway C3 convertase C4b2a. In one embodiment, the complement control protein or fragment of complement control protein is an inhibitor of complement activation steps other than C3 or C5 activation. In various embodiments, the fusion protein comprises a complement receptor 1 (CR1) or a fragment thereof, a membrane cofactor protein (MCP) or a factor thereof, a C4b-binding protein (C4BP) or a fragment thereof, a decay-accelerating factor (DAF) or a fragment thereof, an Apolipoprotein E (ApoE) or a fragment thereof, a factor H protein or a fragment thereof, a human IgG4 or a fragment thereof, a linker, or any combination thereof. In some embodiments, the fragment of factor H comprises SCR domains 1-5 of the factor H protein. In some embodiments, the fragment of decay-accelerating factor (DAF) is the extracellular domain of DAF. In some embodiments, the fragment of CR1 is selected SCRs of the extracellular domain of CR1. In some embodiments, the anti-C5 antibodies described herein are fused (i.e., covalently linked) to factor H or a fragment thereof.

In some embodiments, the anti-C5 antibody fusion protein is an anti-C5 antibody or an antigen binding fragment thereof linked to factor H or a fragment thereof. In some embodiments, the anti-C5 antibody factor H fusion protein comprises any one of the anti-C5 antibody V_{L} sequences described herein. In some embodiments, the anti-C5 antibody is any one of the anti-C5 antibodies described herein. In some embodiments, the factor H is human factor H. In some embodiments, the anti-C5 antibody is fused to the factor H polypeptide or fragment thereof comprises SCR1-5 domains of factor H. In some embodiments, the fragment of factor H comprises SCR domains 1-5 of the factor H protein.

In some embodiments, the anti-C5 antibody factor H fusion protein or a fragment thereof exhibits pH-dependent binding to C5. In some embodiments, the pH-dependent anti-C5 antibody factor H fusion protein or a fragment thereof binds more strongly to C5 at a more neutral pH (e.g., about pH 7.4; such as that found in the blood) than it does at a more acidic pH (e.g., about pH 5.8; such as that found in the endosome).

In some embodiments, anti-C5 antibody factor H fusion protein or a fragment thereof has comparable pH dependence to the anti-C5 antibody. In some embodiments, the anti-C5 antibody factor H fusion protein or a fragment thereof bound to human C5 at pH 7.4 and 25 dissociates from human C5 at pH 5.8 and 25° C. over a time window of 600 seconds in the percentage range of any one of about 5% to about 95%, about 10% to about 90%, about 15% to about 85%, about 20% to about 80%, about 20% to about 75%, about 20% to about 70%, about 20% to about 65%, about 20% to about 60%, about 25% to about 75%, about 25% to about 70%, about 25% to about 65%, about 25% to about 60%, about 30% to about 75%, about 30% to about 70%, about 30% to about 65%, about 30% to about 60%, about 35% to about 75%, about 35% to about 70%, about 35% to about 65%, about 35% to about 60%, about 40% to about 75%, about 40% to about 70%, about 40% to about 65%, about 40% to about 60%. In some embodiments, the anti-C5 antibody factor H fusion protein or a fragment thereof bound to human C5 at pH 7.4 and 25° C. dissociates from human C5 at pH 5.8 and 25° C. in the percentage range of 20% to about 80%.

In some embodiments, the anti-C5 antibody factor H fusion protein or a fragment thereof bound to human C5 at pH 7.4 and 25° C. dissociates from human C5 at pH 7.4 and 25° C. over a time window of 600 seconds in the percentage range of any one of about 0% to about 20%, about 0% to about 18%, about 0% to about 16%, about 0% to about 14%, about 0% to about 12%, about 0% to about 10%, about 0% to about 9%, about 0% to about 8%, about 0% to about 7%, about 0% to about 6%, about 0% to about 5%, about 0% to about 4%, about 0% to about 3%, about 0% to about 2%, about 0% to about 1%. In some embodiments, the anti-C5 antibody factor H fusion protein or a fragment thereof bound to human C5 at pH 7.4 and 25 dissociates from human C5 at pH 7.4 and 25° C. in the percentage range of 0% to about 12%.

In some embodiments, the percentage of dissociation of anti-C5 antibody factor H fusion protein or a fragment thereof at pH 5.8 over the percentage of dissociation of the anti-C5 antibody factor H fusion protein or a fragment thereof at pH 7.4 is any one of 1 or more, 1.5 or more, 2 or more, 2.5 or more, 3 or more, 3.5 or more, 4 or more, 4.5 or more, 5 or more, 6 or more, 7 or more, 8 or more, 9 or more, 10 or more. In some embodiments, the percentage of dissociation of the anti-C5 antibody factor H fusion protein or a fragment thereof at pH 5.8 over the percentage of dissociation of the anti-C5 antibody factor H fusion protein or a fragment thereof at pH 7.4 is 4 or more.

The anti-C5 antibody and the factor H may be linked directly by a single chemical bond (such as peptide bond) or via a peptide linker. The peptide linker may have a naturally occurring sequence, or a non-naturally occurring sequence. For example, a sequence derived from the hinge region of heavy chain-only antibodies may be used as the linker. See, for example, WO1996/34103. In some embodiments, the peptide linker is a flexible linker.

In some embodiments, the fusion protein comprises a fusion protein partner bound to a VH sequence of the antibody. In some embodiments, the fusion protein comprises a fusion protein partner bound to C-terminal of a VH sequence of the antibody. In one embodiment, the fusion protein comprises a fusion protein partner bound to N-terminal of a VH sequence of the antibody.

In some embodiments, the fusion protein comprises a fusion protein partner bound to the Fc of the anti-C5 antibody. The anti-C5 antibody or a fragment thereof may be fused to the factor H or a fragment thereof at either the N-terminus or the C-terminus of the factor H or a fragment thereof. In some embodiments, the anti-C5 antibody or a fragment thereof is fused at the N-terminus of the factor H polypeptide or fragment thereof. In some embodiments, the anti-C5 antibody or a fragment thereof is fused at the C-terminus of the factor H polypeptide or fragment thereof.

In some embodiments, the anti-C5 antibody factor H fusion protein comprises a heavy chain-factor H fusion comprising the amino acid sequence of SEQ ID NO: 10. In some embodiments, the anti-C5 antibody factor H fusion protein comprises a heavy chain-factor H fusion comprising the amino acid sequence of SEQ ID NO: 29.

Properties of the pH-Dependent Anti-C5 Antibody or Fusion Protein Thereof

The anti-C5 antibody or fusion protein described herein are amenable for development and use as a pharmaceutical composition.

The anti-C5 antibody or fusion protein described herein in some embodiments exhibit prolonged serum half-life in vivo. In some embodiments, the anti-C5 antibody or fusion protein exhibits prolonged serum half-life in mice (including transgenic mice). In some embodiments, the anti-C5 antibody or fusion protein exhibits prolonged serum half-life in other test animals. Exemplary test animals include but are not limited to, rats, chickens, rabbits, sheep, and cyno monkeys. In some embodiments, the anti-C5 antibody or fusion protein exhibits prolonged serum half-life in humans. In some embodiments, the anti-C5 antibody or fusion protein has a serum half-life in humans of any one of at least about 2 hrs, about 3 days, about 5 days, about 7 days, about 9 days, about 11 days, about 13 days, about 15 days, about 17 days, about 19 days, about 21 days, about 23 days, about 25 days. In some embodiments, the anti-C5 antibody or fusion protein has a serum half-life in humans that is at least about 25 days.

The anti-C5 antibody or fusion protein described herein may have cross-species reactivity to C5 other than human C5. Without being bound by any theory or hypothesis, cross-reactivity occurs when immunoglobulins from different species share conserved sequences and similar quaternary structure. The paratope (antigen-binding site) of an antibody that recognizes immunoglobulin from one species may detect a homologous epitope on immunoglobulin from another species. This is common in closely related species such as mouse and rat, but may also occur in less obvious pairings. Exemplary non-human C5 include, but are not limited to, mouse C5, rat C5, rabbit C5, sheep C5, cyno monkey C5. In some embodiments, the anti-C5 antibody, or fusion protein may have cross-species reactivity to cyno C5.

The anti-C5 antibody or fusion protein described possesses pH-dependent binding to C5. As used herein, the expression “pH-dependent binding” means that the antibody exhibits reduced binding to C5 at acidic pH (e.g. about pH 5.8; such as that found in the endosome) as compared to its binding at neutral pH (e.g., about pH 7.4; such as that found in the blood).

pH-dependency of the anti-C5 antibody or fusion protein described herein can be determined experimentally by methods known in the art, such as in U.S. Pat. No. 9,079,949, and WO2016/098356. pH-dependency may be reflected in the differences in binding properties such as binding affinity (e.g. dissociation constant), kinetic parameters (e.g. association rate and dissociation rate), and percentage dissociation, at different pH. In some embodiments, the pH-dependency of the anti-C5 antibody or fusion protein described herein may be expressed in terms of the ratio of the percentage dissociation. In some embodiments, the percentage dissociation may be expressed in terms of the low-pH dissociation factor and the neutral-pH dissociation factor.

In some embodiments, the low-pH dissociation factor and the neutral-pH dissociation factor may be measured by methods that allow association and dissociation at different pH, such as biolayer interferometry. The low-pH dissociation factor may be measured by associating antibody and antigen (e.g. anti-C5 antibody and human C5) at pH 7.4 for 600 seconds, followed by a dissociation period in a buffer at pH 5.8 for 600 seconds, and calculation of the percentage of antibody dissociated at pH 5.8 from the antigen. The neutral-pH dissociation factor may be measured by associating antibody and antigen (e.g. anti-C5 antibody and human C5) at pH 7.4 for 600 seconds, followed by a dissociation period in a buffer at pH 7.4 for 600 seconds, and calculation of the percentage of antibody dissociated at pH 7.4 from the antigen.

### III. Pharmaceutical Compositions

Further provided by the present application are pharmaceutical compositions comprising any one of the anti-C5 antibodies or fusion proteins and a pharmaceutically acceptable carrier. Pharmaceutical compositions can be prepared by mixing an anti-C5 antibody having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. It is to be understood that discussion about anti-C5 antibodies in this section also applies to anti-C5 fusion proteins (such as anti-C5-FH fusion proteins).

In some embodiments, the pharmaceutical composition further comprises additional ingredients. Additional ingredients include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, PA), which is incorporated herein by reference.

Additional excipients include agents which can serve as one or more of the following: (1) bulking agents, (2) solubility enhancers, (3) stabilizers and (4) and agents preventing denaturation or adherence to the container wall.

In order for the pharmaceutical compositions to be used for in vivo administration, they must be sterile. The pharmaceutical composition may be rendered sterile by filtration through sterile filtration membranes. The pharmaceutical compositions herein generally are placed into a container having a sterile access port, for example, an intravenous solution bag or vial having a stopper pierceable by a hypodermic injection needle.

Sustained-release preparations may be prepared. Compositions for sustained release or implantation may comprise pharmaceutically acceptable polymeric or hydrophobic materials such as an emulsion, an ion exchange resin, a sparingly soluble polymer, or a sparingly soluble salt. Suitable examples of sustained-release preparations include semi-permeable matrices of solid hydrophobic polymers containing the antagonist, which matrices are in the form of shaped articles, e.g. films, or microcapsules.

The pharmaceutical compositions herein may also contain more than one active compound as necessary for the particular indication being treated, preferably those with complementary activities that do not adversely affect each other. Alternatively, or in addition, the composition may comprise a cytotoxic agent, chemotherapeutic agent, cytokine, immunosuppressive agent, or growth inhibitory agent. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 18th edition.

The formulations of the pharmaceutical compositions may be prepared by any method known or hereafter developed in the art of pharmacology. Preparations include but are not limited to, bringing the active ingredient into association with a carrier or one or more other accessory ingredients, and then, if necessary or desirable, shaping or packaging the product into a desired single- or multi-dose unit. The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and in some embodiments from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container.

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more additional ingredients. Alternately, formulations suitable for buccal administration may comprise a powder or an aerosolized or atomized solution or suspension comprising the active ingredient. In some embodiments, such powdered, aerosolized, or aerosolized formulations, when dispersed, have an average particle or droplet size in the range from about 0.1 to about 200 nanometers, and may further comprise one or more additional ingredients.

### IV. Methods of Use

Also provided herein are methods of inhibiting complement activation and treating diseases (such as complement-mediated diseases or disorders) in an individual by administering an effective amount of the anti-C5 antibody and/or fusion proteins thereof to the individual. In some embodiments, the individual is a human. Although the sections below focus primarily on anti-C5 antibodies, it is to be understood that the methods equally apply to anti-C5 fusion proteins, such as anti-C5-FH fusion proteins.

The anti-C5 antibody or fusion protein thereof can be used in combination with other treatment modalities, such as, for example anti-inflammatory therapies, and the like. Examples of anti-inflammatory therapies that can be used in combination with the methods of the invention include, for example, therapies that employ steroidal drugs, as well as therapies that employ non-steroidal drugs.

In some embodiments, there is provided a method of inhibiting complement activation in an individual, comprising administering (such as systemically administering, for example by subcutaneous or intravenous administration) to the individual an effective amount of an antibody that specifically binds to human C5, wherein the antibody comprises a Y32H mutation, wherein the mutation is in reference to SEQ ID NO:1 under the AbM numbering system, and wherein the antibody binds more strongly to C5 at a neutral pH than it does at an acidic pH. In some embodiments, the VL comprise a Q38R mutation, wherein the mutation is in reference to SEQ ID NO:2 under the AbM numbering system. In some embodiments, the antibody further comprises at least one mutation selected from the group consisting of E27H, N28H, Y30H, T52H, L54H, D56H, T58H, T97H, wherein the mutation is in reference to SEQ ID NO:2 under the AbM numbering system. In some embodiments, the anti-C5 antibody further comprises an Fc region (such as the Fc region of an IgG4, for example an Fc region comprises PLA mutation: S228P, M428L, and N434A). In some embodiments, the anti-C5 antibody cross-reacts with cyno monkey C5. In some embodiments, the anti-C5 antibody binds more strongly at a neutral pH (such as pH 7.4) than it does at an acidic pH (such as pH 5.8). In some embodiments, the low-pH dissociation factor of the anti-C5 antibody is no less than about any of 95%, 90%, 85%, 80%, 75%, 70%, 65%, 60%, 55%, 50%, 45%, 40%, 35%, 30%, 25%, 20%, 15%, 10%, or 5%. In some embodiments, the neutral-pH dissociation factor of the anti-C5 antibody is no more than about any of 20%, 18%, 16%, 14%, 12%, 10%, 9%, 8%, 7%, 6%, 5%, 4%, 3%, 2%, or 1%. In some embodiments, the ratio of the low-pH dissociation factor over the neutral-pH dissociation factor of the anti-C5 antibody is about any of 1 or more, 1.5 or more, 2 or more, 2.5 or more, 3 or more, 3.5 or more, 4 or more, 4.5 or more, 5 or more, 6 or more, 7 or more, 8 or more, 9 or more, 10 or more. In some embodiments, the percentage of dissociation of the antibody for C5 at pH 5.8 over the percentage of dissociation of the antibody for C5 at pH 7.4 is 4 or more. In some embodiments, the anti-C5 antibody is administered by subcutaneous administration.

Inhibition of complement can be assessed, for example, by hemolytic assays. In order to determine the effect of an anti-C5 antibody on classical complement pathway-mediated hemolysis in a serum test solution in vitro, for example, sheep erythrocytes coated with hemolysin or chicken erythrocytes sensitized with anti-chicken erythrocyte antibody are used as target cells. The percentage of lysis is normalized by considering 100% lysis equal to the lysis occurring in the absence of the inhibitor. To determine the effect of anti-C5 antibody on alternative pathway-mediated hemolysis, unsensitized rabbit or guinea pig erythrocytes are used as the target cells. The percentage of lysis is normalized by considering 100% lysis equal to the lysis occurring in the absence of the inhibitor.

In some embodiments, the anti-C5 antibody inhibits complement activation by at least about any of 10%, 20%, 30%, 40%, 50%, 60%, 70%, 80%, 90%, or more.

In some embodiments, there is provided a method of treating a complement-mediated disease in an individual, comprising administering (such as systemically administering, for example by intravenous administration) to the individual an effective amount of an antibody that specifically binds to human C5, wherein the antibody comprises a Y32H mutation, wherein the mutation is in reference to SEQ ID NO:1 under the AbM numbering system, and wherein the antibody binds more strongly to C5 at a neutral pH than it does at an acidic pH. In some embodiments, the VL comprise a Q38R mutation, wherein the mutation is in reference to SEQ ID NO:2 under the AbM numbering system. In some embodiments, the antibody further comprises at least one mutation selected from the group consisting of E27H, N28H, Y30H, T52H, L54H, D56H, T58H, T97H, wherein the mutation is in reference to SEQ ID NO:2 under the AbM numbering system. In some embodiments, the anti-C5 antibody further comprises an Fc region (such as the Fc region of an IgG4, for example an Fc region comprises PLA mutation: S228P, M428L, and N434A). In some embodiments, the anti-C5 antibody cross-reacts with cyno monkey C5. In some embodiments, the anti-C5 antibody binds more strongly at a neutral pH (such as pH 7.4) than it does at an acidic pH (such as pH 5.8). In some embodiments, the low-pH dissociation factor of the anti-C5 antibody is no less than about any of 95%, 90%, 85%, 80%, 75%, 70%, 65%, 60%, 55%, 50%, 45%, 40%, 35%, 30%, 25%, 20%, 15%, 10%, or 5%. In some embodiments, the neutral-pH dissociation factor of the anti-C5 antibody is no more than about any of 20%, 18%, 16%, 14%, 12%, 10%, 9%, 8%, 7%, 6%, 5%, 4%, 3%, 2%, or 1%. In some embodiments, the ratio of the low-pH dissociation factor over the neutral-pH dissociation factor of the anti-C5 antibody is about any of 1 or more, 1.5 or more, 2 or more, 2.5 or more, 3 or more, 3.5 or more, 4 or more, 4.5 or more, 5 or more, 6 or more, 7 or more, 8 or more, 9 or more, 10 or more.

In some embodiments, the complement-mediated disease or disorder is at least selected from the group consisting of: macular degeneration (MD), age-related macular degeneration (AMD), ischemia reperfusion injury, arthritis, rheumatoid arthritis, asthma, allergic asthma, lupus, ulcerative colitis, stroke, post-surgery systemic inflammatory syndrome, asthma, allergic asthma, chronic obstructive pulmonary disease (COPD), paroxysmal nocturnal hemoglobinuria (PNH) syndrome, myasthenia gravis, neuromyelitis optica, (NMO), multiple sclerosis, delayed graft function, antibody-mediated rejection, atypical hemolytic uremic (aHUS) syndrome, central retinal vein occlusion (CRVO), central retinal artery occlusion (CRAO), epidermolysis bullosa, sepsis, organ transplantation, inflammation (including, but not limited to, inflammation associated with cardiopulmonary bypass surgery and kidney dialysis), C3 glomerulopathy, membranous nephropathy, IgA nephropathy, glomerulonephritis (including, but not limited to, anti-neutrophil cytoplasmic antibody (ANCA)-mediated glomerulonephritis, lupus nephritis, and combinations thereof), ANCA-mediated vasculitis, Shiga toxin induced HUS, and antiphospholipid antibody-induced pregnancy loss, COVID-19, or any combinations thereof. In some embodiments, the complement-mediated disease is C3 glomerulopathy.

In some embodiments, the compositions and methods of the invention are useful for treating an individual, including individuals having PNH, who are not adequately responsive to treatment with Eculizumab.

### Dosage and Routes of Administration

Dosages and desired drug concentrations of pharmaceutical compositions of the present application may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles laid down by Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

While this section focuses on anti-C5 antibodies, it is to be understood that the disclosure herein also applies to fusion proteins comprising the anti-C5 antibodies.

Typically, dosages which may be administered in a method of the invention to a subject, in some embodiments a human, range in amount from 0.5 μg to about 50 mg per kilogram of body weight of the subject. While the precise dosage administered will vary depending upon any number of factors, including but not limited to, the type of subject and type of disease state being treated, the age of the subject and the route of administration. In some embodiments, the dosage of the compound will vary from about 1 μg to about 10 mg per kilogram of body weight of the subject. In other embodiments, the dosage will vary from about 3 μg to about 1 mg per kilogram of body weight of the subject.

In some embodiments, the anti-C5 antibody is administered for a single time. In some embodiments, the anti-C5 antibody is administered for multiple times (such as any of 2, 3, 4, 5, 6, or more times). In some embodiments, the anti-C5 antibody is administered once per week, once 2 weeks, once 3 weeks, once 4 weeks, once per month, once per 2 months, once per 3 months, once per 4 months, once per 5 months, once per 6 months, once per 7 months, once per 8 months, once per 9 months, or once per year. In some embodiments, the interval between administrations is about any one of 1 week to 2 weeks, 2 weeks to 1 month, 2 weeks to 2 months, 1 month to 2 months, 1 month to 3 months, 3 months to 6 months, or 6 months to a year. The optimal dosage and treatment regime for a particular patient can readily be determined by one skilled in the art of medicine by monitoring the patient for signs of disease and adjusting the treatment accordingly. The frequency of the dose will be readily apparent to the skilled artisan and will depend upon any number of factors, include but not limited to, the type and severity of the disease being treated, the type and age of the subject, etc.

The anti-C5 antibody of the present application, including but not limited to reconstituted and liquid formulations, are administered to an individual in need of treatment with the anti-C5 antibodies, preferably a human, in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, ophthalmic, rectal, vaginal, parenteral, pulmonary, buccal, intraocular or inhalation routes. Other contemplated formulations include projected nanoparticles, liposomal preparations, resealed erythrocytes containing the active ingredient, and immunologically-based formulations. Parenteral administration of the anti-C5 antibody includes any route of administration characterized by physical breaching of a tissue of an individual and administration of the pharmaceutical composition through the breach in the tissue. Parental administration can be local, regional or systemic. Parenteral administration thus includes, but is not limited to, administration of the anti-C5 antibody by injection of the composition, by application of the composition through a surgical incision, by application of the anti-C5 antibody through a tissue-penetrating non-surgical wound, and the like. In particular, parenteral administration is contemplated to include, but is not limited to, intravenous, intraocular, intravitreal, subcutaneous, intraperitoneal, intramuscular, intradermal, intrasternal injection, and intratumoral.

A anti-C5 antibody of the invention may be prepared, packaged, or sold in bulk, as a single unit dose, or as a plurality of single unit doses. A unit dose is discrete amount of the anti-C5 antibody comprising a predetermined amount of the active ingredient. The amount of the active ingredient is generally equal to the dosage of the active ingredient which would be administered to an individual or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the individual treated and further depending upon the route by which the composition is to be administered. By way of example, the composition may comprise between 0.1% and 100% (w/w) active ingredient. In various embodiments, the composition comprises at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% (w/w) active ingredient.

### V. Methods of Preparation

The present application also provides isolated nucleic acids encoding the anti-C5 antibodies, vectors and host cells comprising such isolated nucleic acids, and recombinant methods for the production of the anti-C5 antibodies. Although the sections below focus on anti-C5 antibodies, it is to be understood that the methods described below also apply to anti-C5 fusion proteins, such as anti-C5-FH fusion proteins.

### Expression Vectors and Cells Producing Antibodies

In some embodiments, the invention is a cell or cell line (such as host cells) that produces at least one of the anti-C5 antibodies or fusion proteins thereof described herein. In one embodiment, the cell or cell line is a genetically modified cell that produces at least one of the anti-C5 antibodies or fusion proteins described herein. In one embodiment, the cell or cell line is a hybridoma that produces at least one of the anti-C5 antibodies or fusion proteins thereof described herein.

Hybrid cells (hybridomas) are generally produced from mass fusions between murine splenocytes, which are highly enriched for B-lymphocytes, and myeloma “fusion partner cells” (Alberts et al., Molecular Biology of the Cell (Garland Publishing, Inc. 1994); Harlow et al., Antibodies. A Laboratory Manual (Cold Spring Harbor Laboratory, Cold Spring Harbor, 1988). The cells in the fusion are subsequently distributed into pools that can be analyzed for the production of antibodies with the desired specificity. Pools that test positive can be further subdivided until single cell clones are identified that produce antibodies of the desired specificity. Antibodies produced by such clones are referred to as monoclonal antibodies.

Also provided are nucleic acids encoding any of the anti-C5 antibodies or fusion proteins thereof disclosed herein, as well as vectors comprising the nucleic acids. Thus, the anti-C5 antibodies or fusion proteins of the invention can be generated by expressing the nucleic acid in a cell or a cell line, such as the cell lines typically used for expression of recombinant or humanized immunoglobulins. Thus, the antibodies and fragments of the invention can also be generated by cloning the nucleic acids into one or more expression vectors, and transforming the vector into a cell line such as the cell lines typically used for expression of recombinant or humanized immunoglobulins.

The genes encoding the heavy and light chains of anti-C5 antibodies or fusion proteins thereof can be engineered according to methods, including but not limited to full length chemical gene synthesis, the polymerase chain reaction (PCR), known in the art (see, e.g., Sambrook et al., Molecular Cloning: A Laboratory Manual, 2nd ed., Cold Spring Harbor, N.Y., 1989; Berger & Kimmel, Methods in Enzymology, Vol. 152: Guide to Molecular Cloning Techniques, Academic Press, Inc., San Diego, Calif., 1987; Co et al., 1992, J. Immunol. 148:1149). For example, genes encoding heavy and light chains, or fragments thereof, can be cloned from an antibody secreting cell's genomic DNA, or cDNA is produced by reverse transcription of the RNA of the cell. Cloning is accomplished by conventional techniques including the use of PCR primers that hybridize to the sequences flanking or overlapping the genes, or segments of genes, to be cloned.

Nucleic acids encoding anti-C5 antibodies or fusion proteins thereof described herein, or the heavy chain or light chain or fragments thereof, can be obtained and used in accordance with recombinant nucleic acid techniques for the production of the specific immunoglobulin, immunoglobulin chain, or a fragment or variant thereof, in a variety of host cells or in an in vitro translation system. For example, the antibody-encoding nucleic acids, or fragments thereof, can be placed into suitable prokaryotic or eukaryotic vectors, e.g., expression vectors, and introduced into a suitable host cell by an appropriate method, e.g., transformation, transfection, electroporation, infection, such that the nucleic acid is operably linked to one or more expression control elements, e.g., in the vector or integrated into the host cell genome.

In some embodiments, the heavy and light chains, or fragments thereof, can be assembled in two different expression vectors that can be used to co-transfect a recipient cell. In some embodiments, each vector can contain two or more selectable genes, one for selection in a bacterial system and one for selection in a eukaryotic system. These vectors allow for the production and amplification of the genes in a bacterial system, and subsequent co-transfection of eukaryotic cells and selection of the co-transfected cells. The selection procedure can be used to select for the expression of antibody nucleic acids introduced on two different DNA vectors into a eukaryotic cell.

Alternatively, the nucleic acids encoding the heavy and light chains, or fragments thereof, may be expressed from one vector. Although the light and heavy chains are coded for by separate genes, they can be joined, using recombinant methods. For example, the two polypeptides can be joined by a synthetic linker that enables them to be made as a single protein chain in which the VL and VH regions pair to form monovalent molecules (known as single chain Fv (scFv); see e.g., Bird et al., 1988, Science 242: 423-426; and Huston et al., 1988, Proc. Natl. Acad. Sci. USA 85:5879-5883).

The invention provides for an isolated nucleic acid molecule comprising a nucleic acid sequence encoding a heavy chain and/or a light chain, as well as fragments thereof. A nucleic acid molecule comprising sequences encoding both the light and heavy chain, or fragments thereof, can be engineered to contain a synthetic signal sequence for secretion of the antibody, or fragment, when produced in a cell. Furthermore, the nucleic acid molecule can contain specific DNA links which allow for the insertion of other antibody sequences and maintain the translational reading frame so to not alter the amino acids normally found in antibody sequences. Exemplary nucleic acids sequences are set for in SEQ ID Nos: 33-62.

In accordance with the present invention, antibody-encoding nucleic acid sequences can be inserted into an appropriate expression vector. In various embodiments, the expression vector comprises the necessary elements for transcription and translation of the inserted antibody-encoding nucleic acid so as to generate recombinant DNA molecules that direct the expression of antibody sequences for the formation of an antibody, or a fragment thereof.

The antibody-encoding nucleic acids, or fragments thereof, can be subjected to various recombinant nucleic acid techniques known to those skilled in the art such as site-directed mutagenesis.

A variety of methods can be used to express nucleic acids in a cell. Nucleic acids can be cloned into a number of types of vectors. However, the present invention should not be construed to be limited to any particular vector. Instead, the present invention should be construed to encompass a wide variety of vectors which are readily available and/or known in the art. For example, the nucleic acid of the invention can be cloned into a vector including, but not limited to a plasmid, a phagemid, a phage derivative, an animal virus, and a cosmid. Vectors of particular interest include expression vectors, replication vectors, probe generation vectors, and sequencing vectors.

In some embodiments, the expression vector is selected from the group consisting of a viral vector, a bacterial vector and a mammalian cell vector. Numerous expression vector systems exist that comprise at least a part or all of the compositions discussed above. Prokaryote- and/or eukaryote-vector based systems can be employed for use with the present invention to produce polynucleotides, or their cognate polypeptides. Many such systems are commercially and widely available.

Viral vector technology is well known in the art and is described, for example, in Sambrook et al. (2012), and in Ausubel et al. (1999), and in other virology and molecular biology manuals. Viruses, which are useful as vectors include, but are not limited to, retroviruses, adenoviruses, adeno-associated viruses, herpes viruses, and lentiviruses. In some embodiments, a murine stem cell virus (MSCV) vector is used to express a desired nucleic acid. MSCV vectors have been demonstrated to efficiently express desired nucleic acids in cells. However, the invention should not be limited to only using a MSCV vector, rather any retroviral expression method is included in the invention. Other examples of viral vectors are those based upon Moloney Murine Leukemia Virus (MoMuLV) and HIV. In some embodiments, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193).

Additional regulatory elements, e.g., enhancers, can be used modulate the frequency of transcriptional initiation. A promoter may be one naturally associated with a gene or nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” e.g., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

A promoter and/or enhancer that effectively directs the expression of the DNA segment in the cell type, organelle, and organism chosen for expression may be employed. Those of skill in the art of molecular biology generally know how to use promoters, enhancers, and cell type combinations for protein expression, for example, see Sambrook et al. (2012). The promoters employed may be constitutive, tissue-specific, inducible, and/or useful under the appropriate conditions to direct high-level expression of the introduced DNA segment, such as is advantageous in the large-scale production of recombinant proteins and fragments thereof.

An example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, Moloney virus promoter, the avian leukemia virus promoter, Epstein-Barr virus immediate early promoter, Rous sarcoma virus promoter, as well as human gene promoters such as, but not limited to, the actin promoter, the myosin promoter, the hemoglobin promoter, and the muscle creatine promoter. Further, the invention should not be limited to the use of constitutive promoters. Inducible promoters are also contemplated as part of the invention. The use of an inducible promoter in the invention provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. Further, the invention includes the use of a tissue-specific promoter or cell-type specific promoter, which is a promoter that is active only in a desired tissue or cell. Tissue-specific promoters are well known in the art and include, but are not limited to, the HER-2 promoter and the PSA associated promoter sequences.

In order to assess the expression of the nucleic acids, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate nucleic acid and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000 FEBS Lett. 479:79-82). Suitable expression systems are well known and may be prepared using well known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing nucleic acids into a cell are known in the art. In the context of an expression vector, the vector can be readily introduced into a host cell, e.g., mammalian, bacterial, yeast or insect cell by any method in the art. For example, the expression vector can be transferred into a host cell by physical, chemical or biological means.

Physical methods for introducing a polynucleotide into a host cell include calcium phosphate precipitation, lipofection, particle bombardment, microinjection, electroporation, laserporation and the like. Methods for producing cells comprising vectors and/or exogenous nucleic acids are well-known in the art. See, for example, Sambrook et al. (2012) and Ausubel et al. (1999).

Biological methods for introducing a nucleic acid of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a nucleic acid into a host cell include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (e.g., an artificial membrane vesicle). The preparation and use of such systems is well known in the art.

Regardless of the method used to introduce exogenous nucleic acids into a host cell or otherwise expose a cell to the nucleic acid of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### 1. Vector Construction

Polynucleotide sequences encoding polypeptide components of the anti-C5 antibody of the present application can be obtained using standard recombinant techniques. Desired polynucleotide sequences may be isolated and sequenced from antibody producing cells such as hybridoma cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the present application. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to: an origin of replication, a selection marker gene, a promoter, a ribosome binding site (RBS), a signal sequence, the heterologous nucleic acid insert and a transcription termination sequence.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is typically transformed using pBR322, a plasmid derived from an E. coli species. pBR322 contains genes encoding ampicillin (Amp) and tetracycline (Tet) resistance and thus provides easy means for identifying transformed cells. pBR322, its derivatives, or other microbial plasmids or bacteriophage may also contain, or be modified to contain, promoters which can be used by the microbial organism for expression of endogenous proteins. Examples of pBR322 derivatives used for expression of particular antibodies are described in detail in Carter et al., U.S. Pat. No. 5,648,237.

In addition, phage vectors containing replicon and control sequences that are compatible with the host microorganism can be used as transforming vectors in connection with these hosts. For example, bacteriophage such as GEM™-11 may be utilized in making a recombinant vector which can be used to transform susceptible host cells such as E. coli LE392.

The expression vector described herein may comprise two or more promoter-cistron pairs, encoding each of the polypeptide components. A promoter is an untranslated regulatory sequence located upstream (5′) to a cistron that modulates its expression. Prokaryotic promoters typically fall into two classes, inducible and constitutive. Inducible promoter is a promoter that initiates increased levels of transcription of the cistron under its control in response to changes in the culture condition, e.g. the presence or absence of a nutrient or a change in temperature.

A large number of promoters recognized by a variety of potential host cells are well known. The selected promoter can be operably linked to cistron DNA encoding the light or heavy chain by removing the promoter from the source DNA via restriction enzyme digestion and inserting the isolated promoter sequence into the vector. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters suitable for use with prokaryotic hosts include the PhoA promoter, the—galactamase and lactose promoter systems, a tryptophan (trp) promoter system and hybrid promoters such as the tac or the trc promoter. However, other promoters that are functional in bacteria (such as other known bacterial or phage promoters) are suitable as well. Their nucleotide sequences have been published, thereby enabling a skilled worker operably to ligate them to cistrons encoding the target light and heavy chains (Siebenlist et al. (1980) Cell 20: 269) using linkers or adaptors to supply any required restriction sites.

In one aspect, each cistron within the recombinant vector comprises a secretion signal sequence component that directs translocation of the expressed polypeptides across a membrane. In general, the signal sequence may be a component of the vector, or it may be a part of the target polypeptide DNA that is inserted into the vector. The signal sequence selected for the purpose of this application should be one that is recognized and processed (i.e. cleaved by a signal peptidase) by the host cell. For prokaryotic host cells that do not recognize and process the signal sequences native to the heterologous polypeptides, the signal sequence is substituted by a prokaryotic signal sequence selected, for example, from the group consisting of the alkaline phosphatase, penicillinase, Ipp, or heat-stable enterotoxin II (STII) leaders, LamB, PhoE, PelB, OmpA and MBP. In some embodiments, the signal sequences used in both cistrons of the expression system are STII signal sequences or variants thereof.

In some embodiments, the production of the anti-C5 antibodies can occur in the cytoplasm of the host cell, and therefore does not require the presence of secretion signal sequences within each cistron. In some embodiments, polypeptide components, such as the polypeptide encoding the VH domain of the first antigen binding portion optionally fused to the second antigen binding portion, and the polypeptide encoding the V_{L} domain of the first antigen binding portion optionally fused to the second antigen binding portion, are expressed, folded and assembled to form functional anti-C5 antibodies within the cytoplasm. Certain host strains (e.g., the E. coli trxB⁻ strains) provide cytoplasm conditions that are favorable for disulfide bond formation, thereby permitting proper folding and assembly of expressed protein subunits. Proba and Pluckthun Gene, 159:203 (1995).

### 2. Protein Production in Prokaryotic Host Cells.

Prokaryotic host cells suitable for expressing the anti-C5 antibodies of the present application include Archaebacteria and Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus. In some embodiments, gram-negative cells are used. In some embodiments, E. coli cells are used as hosts. Examples of E. coli strains include strain W3110 (Bachmann, Cellular and Molecular Biology, vol. 2 (Washington, D.C.: American Society for Microbiology, 1987), pp. 1190-1219; ATCC Deposit No. 27,325) and derivatives thereof, including strain 33D3 having genotype W3110 AfhuA (AtonA) ptr3 lac Iq lacL8 AompT A(nmpc-fepE) degP41 kan^{R} (U.S. Pat. No. 5,639,635). Other strains and derivatives thereof, such as E. coli 294 (ATCC 31,446), E. coli B, E. coli 1776 (ATCC 31,537) and E. coli RV308 (ATCC 31,608) are also suitable. These examples are illustrative rather than limiting. Methods for constructing derivatives of any of the above-mentioned bacteria having defined genotypes are known in the art and described in, for example, Bass et al., Proteins, 8:309-314 (1990). It is generally necessary to select the appropriate bacteria taking into consideration replicability of the replicon in the cells of a bacterium. For example, E. coli, Serratia, or Salmonella species can be suitably used as the host when well-known plasmids such as pBR322, pBR325, pACYC177, or pKN410 are used to supply the replicon.

Typically the host cell should secrete minimal amounts of proteolytic enzymes, and additional protease inhibitors may desirably be incorporated in the cell culture.

Host cells are transformed with the above-described expression vectors and cultured in conventional nutrient media modified as appropriate for inducing promoters, selecting transformants, or amplifying the genes encoding the desired sequences. Transformation means introducing DNA into the prokaryotic host so that the DNA is replicable, either as an extrachromosomal element or by chromosomal integrant. Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

Prokaryotic cells used to produce the anti-C5 antibodies of the present application are grown in media known in the art and suitable for culture of the selected host cells. Examples of suitable media include luria broth (LB) plus necessary nutrient supplements. In some embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol and dithiothreitol.

The prokaryotic host cells are cultured at suitable temperatures. For E. coli growth, for example, the preferred temperature ranges from about 20° C. to about 39° C., more preferably from about 25° C. to about 37° C., even more preferably at about 30° C. The pH of the medium may be any pH ranging from about 5 to about 9, depending mainly on the host organism. For E. coli, the pH is preferably from about 6.8 to about 7.4, and more preferably about 7.0.

If an inducible promoter is used in the expression vector, protein expression is induced under conditions suitable for the activation of the promoter. In some embodiments, PhoA promoters are used for controlling transcription of the polypeptides. Accordingly, the transformed host cells are cultured in a phosphate-limiting medium for induction. Preferably, the phosphate-limiting medium is the C.R.A.P medium (see, e.g., Simmons et al., J. Immunol. Methods (2002), 263:133-147). A variety of other inducers may be used, according to the vector construct employed, as is known in the art.

The expressed anti-C5 antibodies of the present application are secreted into and recovered from the periplasm of the host cells. Protein recovery typically involves disrupting the microorganism, generally by such means as osmotic shock, sonication or lysis. Once cells are disrupted, cell debris or whole cells may be removed by centrifugation or filtration. The proteins may be further purified, for example, by affinity resin chromatography. Alternatively, proteins can be transported into the culture media and isolated therein. Cells may be removed from the culture and the culture supernatant being filtered and concentrated for further purification of the proteins produced. The expressed polypeptides can be further isolated and identified using commonly known methods such as polyacrylamide gel electrophoresis (PAGE) and Western blot assay.

Alternatively, protein production is conducted in large quantity by a fermentation process. Various large-scale fed-batch fermentation procedures are available for production of recombinant proteins. Large-scale fermentations have at least 1000 liters of capacity, preferably about 1,000 to 100,000 liters of capacity. These fermentors use agitator impellers to distribute oxygen and nutrients, especially glucose (the preferred carbon/energy source). Small scale fermentation refers generally to fermentation in a fermentor that is no more than approximately 100 liters in volumetric capacity, and can range from about 1 liter to about 100 liters.

During the fermentation process, induction of protein expression is typically initiated after the cells have been grown under suitable conditions to a desired density, e.g., an OD₅₅₀ of about 180-220, at which stage the cells are in the early stationary phase. A variety of inducers may be used, according to the vector construct employed, as is known in the art and described above. Cells may be grown for shorter periods prior to induction. Cells are usually induced for about 12-50 hours, although longer or shorter induction time may be used.

To improve the production yield and quality of the anti-C5 antibodies of the present application, various fermentation conditions can be modified. For example, to improve the proper assembly and folding of the secreted polypeptides, additional vectors overexpressing chaperone proteins, such as Dsb proteins (DsbA, DsbB, DsbC, DsbD and or DsbG) or FkpA (a peptidylprolyl cis,trans-isomerase with chaperone activity) can be used to co-transform the host prokaryotic cells. The chaperone proteins have been demonstrated to facilitate the proper folding and solubility of heterologous proteins produced in bacterial host cells. Chen et al. (1999) J Bio Chem 274:19601-19605; Georgiou et al., U.S. Pat. No. 6,083,715; Georgiou et al., U.S. Pat. No. 6,027,888; Bothmann and Pluckthun (2000) J. Biol. Chem. 275:17100-17105; Ramm and Pluckthun (2000) J. Biol. Chem. 275:17106-17113; Arie et al. (2001) Mol. Microbiol. 39:199-210.

To minimize proteolysis of expressed heterologous proteins (especially those that are proteolytically sensitive), certain host strains deficient for proteolytic enzymes can be used for the present application. For example, host cell strains may be modified to effect genetic mutation(s) in the genes encoding known bacterial proteases such as Protease III, OmpT, DegP, Tsp, Protease I, Protease Mi, Protease V, Protease VI and combinations thereof. Some E. coli protease-deficient strains are available and described in, for example, Joly et al. (1998), supra; Georgiou et al., U.S. Pat. No. 5,264,365; Georgiou et al., U.S. Pat. No. 5,508,192; Hara et al., Microbial Drug Resistance, 2:63-72 (1996).

E. coli strains deficient for proteolytic enzymes and transformed with plasmids overexpressing one or more chaperone proteins may be used as host cells in the expression system encoding the anti-C5 antibodies of the present application.

### 3. Protein Production in Eukaryotic Cells

In some embodiments, the anti-C5 antibodies or fusion proteins described herein can be expressed in eukaryotic cells. For eukaryotic expression, the vector components generally include, but are not limited to, one or more of the following, a signal sequence, an origin of replication, one or more marker genes, and enhancer element, a promoter, and a transcription termination sequence.

### a) Selection and Transformation of Host Cells

Suitable host cells for cloning or expressing the DNA in the vectors herein include higher eukaryote cells described herein, including vertebrate host cells. Propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/−DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for anti-C5 antibodies production and cultured in conventional nutrient media modified as appropriate for inducing promoters, selecting transformants, or amplifying the genes encoding the desired sequences. In some embodiments, the anti-C5 antibodies or fusion proteins are expressed in CHO cells. In some embodiments, the anti-C5 antibodies or fusion proteins are expressed in Expi-CHO cells.

### b) Culturing the Host Cells

The host cells used to produce the anti-C5 antibodies of the present application may be cultured in a variety of media. Commercially available media such as Ham's F10 (Sigma), Minimal Essential Medium (MEM), (Sigma), RPMI-1640 (Sigma), and Dulbecco's Modified Eagle's Medium (DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

### c) Protein Purification

The anti-C5 antibodies produced herein may be further purified to obtain preparations that are substantially homogeneous for further assays and uses. Standard protein purification methods known in the art can be employed.

### 4. Antibody Production and Modification

Components of the anti-C5 antibodies can be produced using any known methods in the art, including methods described below.

### a) Monoclonal Antibodies

Monoclonal antibodies are obtained from a population of substantially homogeneous antibodies, i.e., the individual antibodies comprising the population are identical except for possible naturally occurring mutations and/or post-translational modifications (e.g., isomerizations, amidations) that may be present in minor amounts. Thus, the modifier “monoclonal” indicates the character of the antibody as not being a mixture of discrete antibodies.

For example, the monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as hereinabove described to elicit lymphocytes that produce or are capable of producing antibodies that will specifically bind the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986).

The immunizing agent will typically include the antigenic protein or a fusion variant thereof. Generally either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. Goding, Monoclonal Antibodies: Principles and Practice, Academic Press (1986), pp. 59-103.

Immortalized cell lines are usually transformed mammalian cells, particularly myeloma cells of rodent, bovine and human origin. Usually, rat or mouse myeloma cell lines are employed. The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which are substances that prevent the growth of HGPRT-deficient cells.

Preferred immortalized myeloma cells are those that fuse efficiently, support stable high-level production of antibody by the selected antibody-producing cells, and are sensitive to a medium such as HAT medium. Among these, preferred are murine myeloma lines, such as those derived from MOPC-21 and MPC-11 mouse tumors available from the Salk Institute Cell Distribution Center, San Diego, Calif. USA, and SP-2 cells (and derivatives thereof, e.g., X63-Ag8-653) available from the American Type Culture Collection, Manassas, Va. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The culture medium in which the hybridoma cells are cultured can be assayed for the presence of monoclonal antibodies directed against the desired antigen. Preferably, the binding affinity and specificity of the monoclonal antibody can be determined by immunoprecipitation or by an in vitro binding assay, such as radioimmunoassay (RIA) or enzyme-linked assay (ELISA). Such techniques and assays are known in the in art. For example, binding affinity may be determined by the Scatchard analysis of Munson et al., Anal. Biochem, 107:220 (1980).

After hybridoma cells are identified that produce antibodies of the desired specificity, affinity, and/or activity, the clones may be subcloned by limiting dilution procedures and grown by standard methods (coding, supra). Suitable culture media for this purpose include, for example, D-MEM or RPMI-1640 medium. In addition, the hybridoma cells may be grown in vivo as tumors in a mammal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Monoclonal antibodies may also be made by recombinant DNA methods, such as those described in U.S. Pat. No. 4,816,567, and as described above. DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, in order to synthesize monoclonal antibodies in such recombinant host cells. Review articles on recombinant expression in bacteria of DNA encoding the antibody include Skerra et al., Curr. Opinion in Immunol., 5:256-262 (1993) and Plückthun, Immunol Revs. 130:151-188 (1992).

In a further embodiment, antibodies can be isolated from antibody phage libraries generated using the techniques described in McCafferty et al., Nature, 348:552-554 (1990). Clackson el al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nucl. Acids Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al., Proc. Natl Acad. Sci. USA, 81:6851 (1984)), or by covalently joining to the immunoglobulin coding sequence all or part of the coding sequence for a non-immunoglobulin polypeptide. Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

The monoclonal antibodies described herein may by monovalent, the preparation of which is well known in the art. For example, one method involves recombinant expression of immunoglobulin light chain and a modified heavy chain. The heavy chain is truncated generally at any point in the Fc region so as to prevent heavy chain crosslinking. Alternatively, the relevant cysteine residues may be substituted with another amino acid residue or are deleted so as to prevent crosslinking. In vitro methods are also suitable for preparing monovalent antibodies. Digestion of antibodies to produce fragments thereof, particularly Fab fragments, can be accomplished using routine techniques known in the art.

Chimeric or hybrid antibodies also may be prepared in vitro using known methods in synthetic protein chemistry, including those involving crosslinking agents. For example, immunotoxins may be constructed using a disulfide-exchange reaction or by forming a thioether bond. Examples of suitable reagents for this purpose include iminothiolate and methyl-4-mercaptobutyrimidate.

### b) Humanized Antibodies

The anti-C5 antibodies or fusion proteins thereof may further comprise humanized or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementarity determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domain, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin. Jones et al., Nature 321: 522-525 (1986); Riechmann et al., Nature 332: 323-329 (1988) and Presta, Curr. Opin. Struct. Biol. 2: 593-596 (1992).

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers, Jones et al., Nature 321:522-525 (1986); Riechmann et al., Nature 332:323-327 (1988); Verhoeyen et al., Science 239:1534-1536 (1988), or through substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody. Sims et al., J. Immunol., 151:2296 (1993); Chothia et al., J. Mol. Biol., 196:901 (1987). Another method uses a particular framework derived from the consensus sequence of all human antibodies of a particular subgroup of light or heavy chains. The same framework may be used for several different humanized antibodies. Carter et al., Proc. Natl. Acad. Sci. USA, 89:4285 (1992); Presta et al., J. Immunol., 151:2623 (1993).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the CDR residues are directly and most substantially involved in influencing antigen binding.

Various forms of the humanized antibody are contemplated. For example, the humanized antibody may be an antibody fragment, such as an Fab, which is optionally conjugated with one or more cytotoxic agent(s) in order to generate an immunoconjugate. Alternatively, the humanized antibody may be an intact antibody, such as an intact IgG1 antibody.

### c) Human Antibodies

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl, Acad. Sci. USA, 90:2551 (1993); Jakobovits et al., Nature, 362:255-258 (1993), Bruggermann et al., Year in Immuno., 7:33 (1993); U.S. Pat. No. 5,591,669 and WO 97/17852. Transgenic mice or rats capable of producing fully human antibodies are known in the art. See, e.g., US20090307787A1, U.S. Pat. No. 8,754,287, US20150289489A1, US20100122358A1, and WO2004049794.

Alternatively, phage display technology can be used to produce human antibodies and antibody fragments in vitro, from immunoglobulin variable (V) domain gene repertoires from unimmunized donors. McCafferty et al., Nature 348:552-553 (1990); Hoogenboom and Winter, J. Mol. Biol. 227: 381 (1991). According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, Kevin S, and Chiswell, David J., Curr. Opin Struct. Biol. 3:564-571 (1993). Several sources of V-gene segments can be used for phage display. Clackson et al., Nature 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993), See also, U.S. Pat. Nos. 5,565,332 and 5,573,905.

The techniques of Cole et al., and Boerner et al., are also available for the preparation of human monoclonal antibodies (Cole et al., Monoclonal Antibodies and Cancer Therapy, Alan R. Liss, p. 77 (1985) and Boerner et al., J. Immunol. 147(1): 86-95 (1991). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly and antibody repertoire. This approach is described, for example, in U.S. Pat. Nos. 5,545,807; 5,545,806, 5,569,825, 5,625,126, 5,633,425, 5,661,016 and in the following scientific publications: Marks et al., Bio/Technology 10: 779-783 (1992); Lonberg et al., Nature 368: 856-859 (1994); Morrison, Nature 368: 812-13 (1994), Fishwild et al., Nature Biotechnology 14: 845-51 (1996), Neuberger, Nature Biotechnology 14: 826 (1996) and Lonberg and Huszar, Intern. Rev. Immunol. 13: 65-93 (1995).

Finally, human antibodies may also be generated in vitro by activated B cells (see U.S. Pat. Nos. 5,567,610 and 5,229,275).

### d) Antibody Fragments

In certain circumstances there are advantages to using antibody fragments, such as antigen binding fragments, rather than whole antibodies. Smaller fragment sizes allow for rapid clearance, and may lead to improved access to solid tumors.

Various techniques have been developed for the production of antibody fragments. Traditionally, these fragments were derived via proteolytic digestion of intact antibodies (see, e.g., Morimoto et al., J Biochem Biophys. Method. 24:107-117 (1992); and Brennan et al., Science 229:81 (1985)). However, these fragments can now be produced directly by recombinant host cells. Fab, Fv and scFv antibody fragments can all be expressed in and secreted from E thus allowing the facile production of large amounts of these fragments. Antibody fragments can be isolated from the antibody phage libraries discussed above. Alternatively, Fab′-SH fragments can be directly recovered from E. coli and chemically coupled to form F(ab)₂ fragments (Carter et al., Bio/Technology 10:163-167 (1992)). According to another approach, F(ab′)₂ fragments can be isolated directly from recombinant host cell culture. Fab and F(ab′)₂ with increase in vivo half-life is described in U.S. Pat. No. 5,869,046. In other embodiments, the antibody of choice is a single chain Fv fragment (scFv). See WO 93/16185; U.S. Pat. Nos. 5,571,894 and 5,587,458. The antibody fragment may also be a “linear antibody”, e.g., as described in U.S. Pat. No. 5,641,870. Such linear antibody fragments may be monospecific or bispecific.

### e) Effector Function Engineering

It may be desirable to modify the anti-C5 antibodies of the present application with respect to Fc effector function, e.g., so as to modify enhance or eliminate) antigen-dependent cell-mediated cytotoxicity (ADCC) and/or complement dependent cytotoxicity (CDC) of the antibody. In a preferred embodiment, Fe effector function of the anti-C5 antibody is reduced or eliminated: This may be achieved by introducing one or more amino acid substitutions in an Fc region of the antibody. Alternatively or additionally, cysteine residue(s) may be introduced in the Fc region, thereby allowing interchain disulfide bond formation in this region. The homodimeric anti-C5 antibody thus generated may have improved internalization capability and/or increased complement-mediated cell killing and antibody-dependent cellular cytotoxicity (ADCC). See Caron et al., J. Exp. Med 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al., Cancer Research 53:2560-2565 (1993). Alternatively, an antibody can be engineered which has dual Fe regions and may thereby have enhanced complement lysis and ADCC capabilities. See Stevenson et al., Anti-Cancer Drug Design 3:219-230 (1989).

To increase the serum half-life of the antibody, one may incorporate a salvage receptor binding epitope into the anti-C5 antibody as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

### f) Other Amino Acid Sequence Modifications

Amino acid sequence modification(s) of the antibodies, such as single chain antibodies or antibody components of the anti-C5 antibodies, described herein are contemplated. For example, it may be desirable to improve the binding affinity and/or other biological properties of the antibody. Amino acid sequence variants of the antibody are prepared by introducing appropriate nucleotide changes into the antibody nucleic acid, or by peptide synthesis. Such modifications include, for example, deletions from, and/or insertions into and/or substitutions of, residues within the amino acid sequences of the antibody. Any combination of deletion, insertion, and substitution is made to arrive at the final construct, provided that the final construct possesses the desired characteristics. The amino acid changes also may alter post-translational processes of the antibody, such as changing the number or position of glycosylation sites.

A useful method for identification of certain residues or regions of the antibody that are preferred locations for mutagenesis is called “alanine scanning mutagenesis” as described by Cunningham and Wells in Science, 244:1081-1085 (1989), Here, a residue or group of target residues are identified (e.g., charged residues such as Arg, Asp, His, Lys, and Glu) and replaced by a neutral or negatively charged amino acid (most preferably alanine or polyalanine) to affect the interaction of the amino acids antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed antibody variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of the antibody molecule include the fusion to the N- or C-terminus of the antibody to an enzyme (e.g. for ADEPT) or a polypeptide which increases the serum half-life of the antibody.

Another type of variant is an amino acid substitution variant. These variants have at least one amino acid residue in the antibody molecule replaced by a different residue. The sites of greatest interest for substitutional mutagenesis include the hypervariable regions, but FR alterations are also contemplated. Conservative substitutions are shown in the table below under the heading of “preferred substitutions”. If such substitutions result in a change in biological activity, then more substantial changes, denominated “exemplary substitutions” in Table 1, or as further described below in reference to amino acid classes, may be introduced and the products screened.

TABLE 1
Amino Acid Substitutions

| | | Preferred |
|---|---|---|
| Original Residue | Exemplary Substitutions | Substitutions |
| Ala (A) | val; leu; ile | val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; norleucine | leu |
| Leu (L) | norleucine; ile; val; met; ala; phe | ile |
| Lys (K) | arg; gln; asn | arg |
| Met (M) | leu; phe; ile | leu |
| Phe (F) | leu; val; ile; ala; tyr | tyr |
| Pro (P) | Ala | ala |
| Ser (S) | Thr | thr |
| Thr (T) | Ser | ser |
| Trp (W) | tyr; phe | tyr |
| Tyr (Y) | trp; phe; thr; ser | phe |
| Val (V) | ile; leu; met; phe; ala; norleucine | leu |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gin, his, lys, arg;
(5) residues that influence chain orientation: gly, pro; and
(6) aromatic: trp, tyr, phe.

Non-conservative substitutions will entail exchanging a member of one of these classes for another class.

Any cysteine residue not involved in maintaining the proper conformation of the antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

A particularly preferred type of substitutional variant involves substituting one or more hypervariable region residues of a parent antibody (e.g. a humanized or human antibody). Generally, the resulting variant(s) selected for further development will have improved biological properties relative to the parent antibody from which they are generated.

Another type of amino acid variant of the antibody alters the original glycosylation pattern of the antibody. By altering is meant deleting one or more carbohydrate moieties found in the antibody, and/or adding one or more glycosylation sites that are not present in the antibody.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites), The alteration may also be made by the addition of, or substitution by, one or more serine or threonine residues to the sequence of the original antibody (for O-linked glycosylation sites).

Nucleic acid molecules encoding amino acid sequence variants to the anti-C5 antibodies of the present application are prepared by a variety of methods known in the art. These methods include, but are not limited to, isolation from a natural source (in the case of naturally occurring amino acid sequence variants) or preparation by oligonucleotide-mediated (or site-directed) mutagenesis, PC R mutagenesis, and cassette mutagenesis of an earlier prepared variant or a non-variant versions.

### g) Other Modifications

The anti-C5 antibodies of the present application can be further modified to contain additional nonproteinaceous moieties that are known in the art and readily available. Preferably, the moieties suitable for derivatization of the antibody are water-soluble polymers, Non-limiting examples of water-soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc. Such techniques and other suitable formulations are disclosed in Remington: The Science and Practice Pharmacy, 20th Ed., Alfonso Gennaro, Philadelphia College of Pharmacy and Science (2000).

### Kits

The invention also includes a kit comprising an anti-C5 antibody (or anti-C5 fusion proteins) of the invention and an instructional material which describes, for instance, administering the anti-C5 antibody (or anti-C5 fusion proteins) to an individual as a therapeutic treatment or a non-treatment use as described elsewhere herein. In an embodiment, this kit further comprises a (optionally sterile) pharmaceutically acceptable carrier suitable for dissolving or suspending therapeutic composition, comprising an anti-C5 antibody, or combinations thereof, of the invention, for instance, prior to administering the antibody to an individual. Optionally, the kit comprises an applicator for administering the antibody. Also provided are unit dosage forms comprising the anti-C5 antibodies (or anti-C5 fusion proteins).

### EXAMPLES

The examples below are intended to be purely exemplary of the invention and should therefore not be considered to limit the invention in any way. The following examples and detailed description are offered by way of illustration and not by way of limitation.

### Example 1: Construction, Expression and Purification of the Anti-C5 Antibodies

An anti-C5 IgG4 antibody comprising the VH amino acid sequence of SEQ ID NO: 1 and VL amino acid sequence of SEQ ID NO:2 was used to generate mutant antibodies. A point mutation Y32H was introduced into the VH (SEQ ID NO:9). Another point mutation Q38R was also introduced into the VL (SEQ ID NO:18). In addition, three Fc domain mutations (S228P, M428L and N434A, herein referred to as PLA mutations, SEQ ID NO:27) were introduced into the mutant anti-C5 antibodies. The designed mutations targeted positions were introduced by QuikChange® (Agilent) or Q5® Site-Directed Mutagenesis (NEB). The mutant antibody comprising Y32H/Q38R and the PLA mutations was denoted as Y32H/Q38R. To selectively increase C5 binding dissociation at pH 5.8 while maintaining Y32H/Q38R high binding affinity at pH 7.4, additional Histidine mutation(s) in the targeted VL CDR region of Y32H/Q38R were constructed further by QuikChange® mutagenesis. For each variant, HEK293 cells were co-transfected with the expression plasmids of Y32H/Q38R heavy chain and a new light chain transiently and cultivated with serum-free medium in shaking flasks for 5-7 days. The expression supernatant was collected by centrifugation for binding characterization. Mutant protein were further purified from the expression supernatant by Protein-A affinity chromatography for affinity determinations. Purified protein was dialyzed into 20 mM Histidine buffer with 150 mM NaCl and quantitated by Nanodrop (Thermo Fisher Scientific, Inc., USA). before affinity measurement using Gator™ (Probe Life, Inc.). From 28 number of variants, 7 constructs were identified as having superior binding compared to the antibody comprising an amino acid sequence of SEQ ID NO:18. All 7 constructs have a single mutation Y32H in VH CDR1, a mutation Q38R in frame 2 of VL, PLA mutations in the Fc, and further comprise a mutation in the VL CDRs: E27H (VL CDR1, position 4, denoted Y32H/Q38R/E27H, VL sequence in SEQ ID NO:19), N28H (VL CDR1, position 5, denoted Y32H/Q38R/N28H, VL sequence in SEQ ID NO:20), Y30H (VL CDR1, position 7, denoted Y32H/Q38R/Y30H, VL sequence in SEQ ID NO:21), T52H (VL CDR2, position 4, denoted Y32H/Q38R/T52H, VL sequence in SEQ ID NO:22), L54H (VL CDR2, position 6, denoted Y32H/Q38R/L54H, VL sequence in SEQ ID NO:23), D56H (VL CDR2, position 8, denoted Y32H/Q38R/D56H, VL sequence in SEQ ID NO:24) or T97H (VL CDR3, position 9, denoted Y32H/Q38R/T97H, VL sequence in SEQ ID NO:25).

In addition, Y32H/Q38R and the 7 histidine mutation constructs were further fused to factor H SCR1-5 to generate anti-C5 mAb factor fusion variants. Factor H SCR1-5 was fused to the C terminus of the Fc. Fusion proteins were denoted as Y32H/Q38R-fH (fH fused with Y32H/Q38R), Y32H/Q38R/E27H-fH (fH fused with Y32H/Q38R/E27H), etc. Both anti-C5 antibodies and anti-C5 antibody-factor H fusion proteins were evaluated for their binding capacities, in vivo activities, and PK/PD properties.

### Example 2: In Vitro Binding Assays

1. pH Dependent Binding Assays with the Anti-C5 Antibodies

The 8 histidine mutation-containing anti-C5 antibodies, including Y32H/Q38R, were tested for their pH dependence in C5 binding assays. ALXN1210, a pH-dependent binding variant of Eculizumab (U.S. Pat. No. 9,079,949) was used as a reference. A Biolayer interferometry instrument, Gator™ (Probe life Inc., USA) was used to determine pH dependent dissociation of histidine mutants and the benchmark antibodies. Briefly, anti-human IgG Fc (FHC) Probes were equilibrated in the kinetic (K) buffer of Phosphate-buffered saline containing 0.02% bovine serum albumin and 0.002% Tween20 pH 7.4. Antibodies were captured onto the sensors by dipping them into 200 μL of transfection supernatant for 600 s at pH 7.4. The biosensors were then incubated with human C5 prepared in the K buffer pH 7.4 (40 nM) for 600 s followed by 600-second dissociation period in K Buffer, pH 7.4 or pH 5.8. The data were processed and analyzed by the Gator evaluation software.

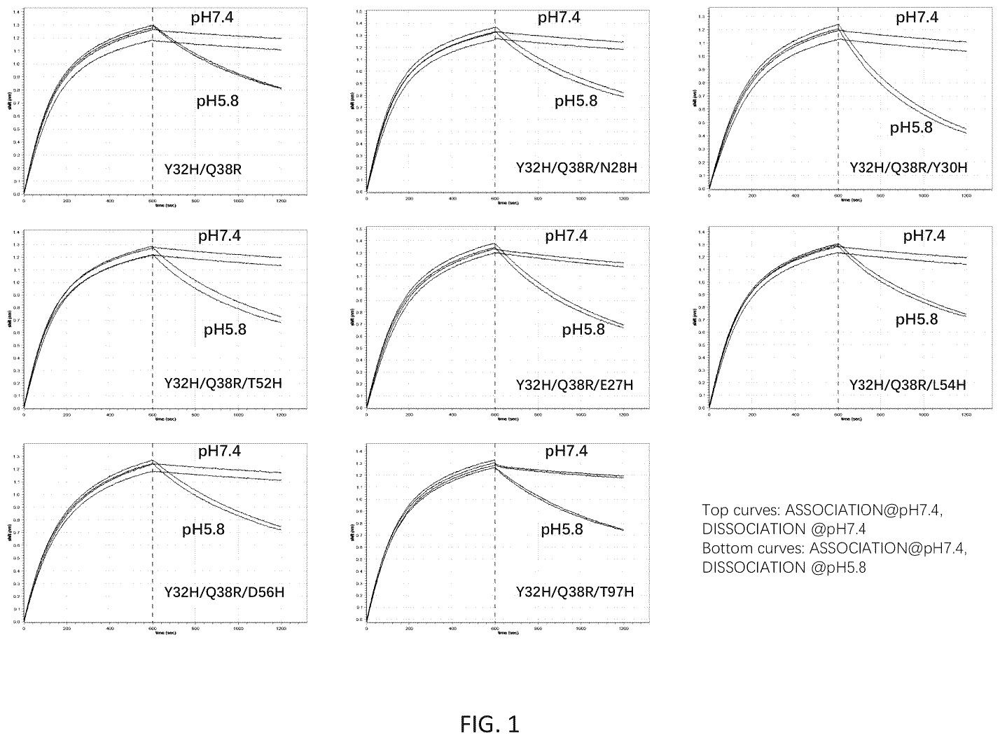

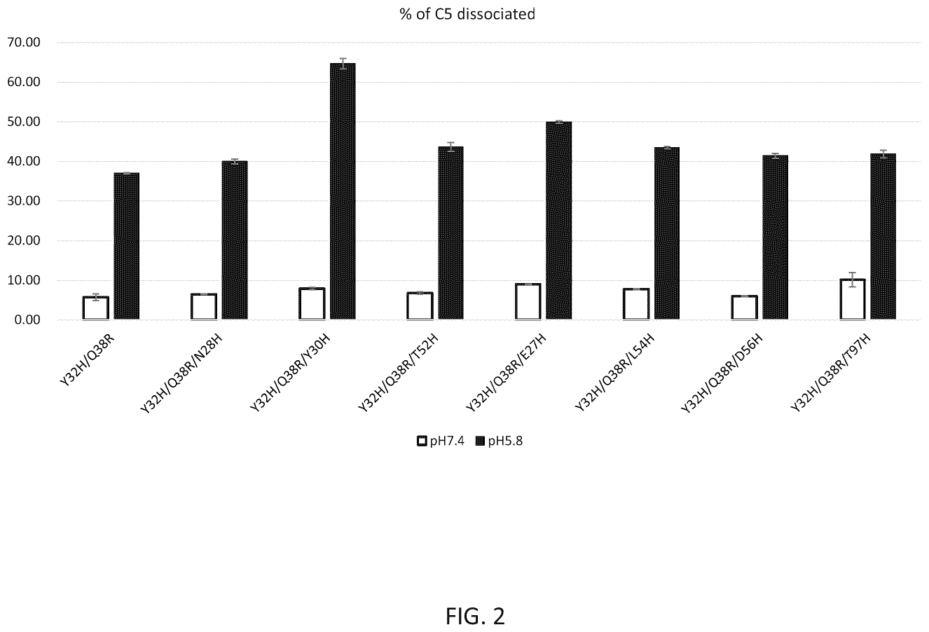

Binding curves of 8 histidine mutants to human C5a e shown in FIG. 1. These histidine mutants all displayed pH-dependent human C5 binding, with higher dissociation at pH 5.8, as shown in FIGS. 1 and 2. Among the 8 histidine mutants, Y32H/Q38R has the least dissociation at pH 5.8 whereas The experiment was repeated and the results are summarized in Table 2.

TABLE 2
pH dependence of human C5 binding to anti-C5 antibodies
% Dissociation at 600 s

| Sample | pH 7.4, n = 2 | pH 5.8, n = 2 |
|---|---|---|
| Y32H/Q38R | 5.69 ± 0.86 | 36.97 ± 0.17 |
| Y32H/Q38R/N28H | 6.44 ± 0.09 | 39.95 ± 0.64 |
| Y32H/Q38R/Y30H | 7.87 ± 0.31 | 64.59 ± 1.33 |
| Y32H/Q38R/T52H | 6.73 ± 0.32 | 43.60 ± 1.07 |
| Y32H/Q38R/E27H | 8.96 ± 0.01 | 49.86 ± 0.33 |
| Y32H/Q38R/L54H | 7.76 ± 0.13 | 43.41 ± 0.29 |
| Y32H/Q38R/D56H | 5.97 ± 0.15 | 41.35 ± 0.61 |
| Y32H/Q38R/T97H | 10.09 ± 1.82 | 41.81 ± 0.98 |

2. Comparison of the pH-Dependent Binding Properties of Y32H/Q38R/L54H, Eculizumab and ALXN1210

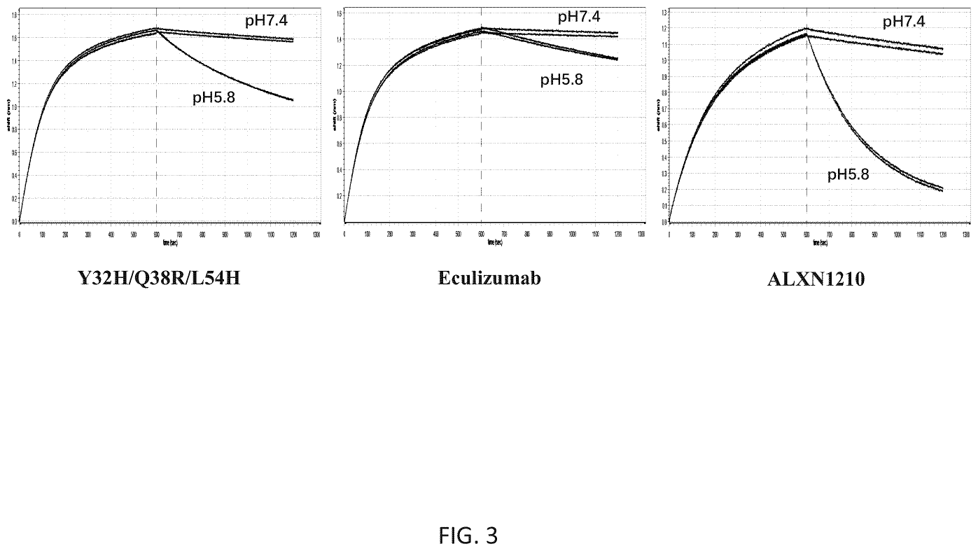

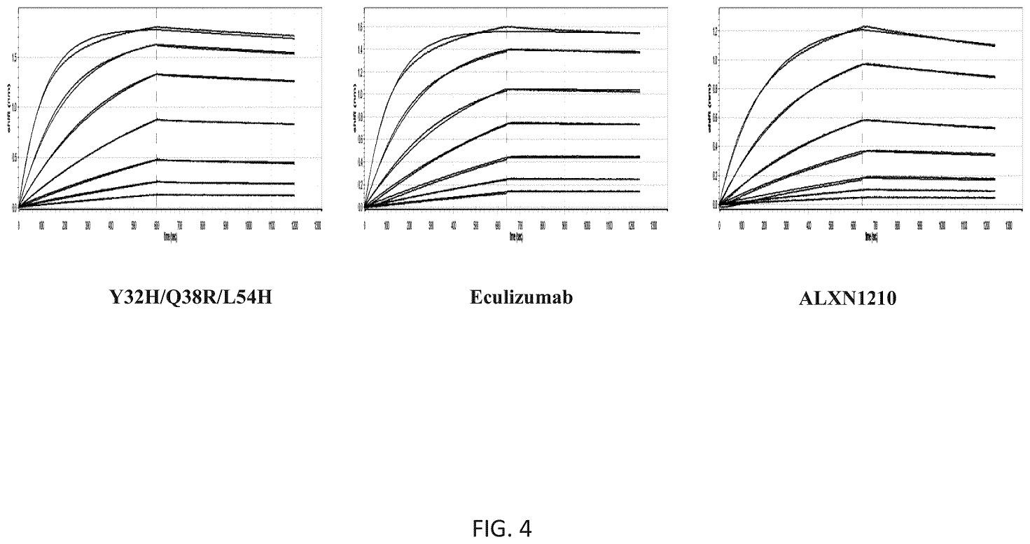

Experiment was performed to compare the pH-dependent C5 binding properties of Y32H/Q38R/L54H, Eculizumab and ALXN1210. C5 binding to probe-immobilized antibodies was allowed proceed at pH 7.4 for 600 s, followed by a 600 s dissociation period in K Buffer, pH 7.4 or pH 5.8. The data were processed and analyzed by the Gator evaluation software. FIG. 3 and Table 3 show that Y32H/Q38R/L54H has significantly greater dissociation at pH 5.8 compared to Eculizumab, but less dissociation at pH 5.8 compared with ALXN1210. Conversely, Y32H/Q38R/L54H has less dissociation at pH 7.4 than ALXN1210 and faster dissociation at pH 7.4 than Eculizumab. Rate constants and affinities of Y32H/Q38R/L54H, Eculizumab and ALXN1210 binding to human C5 were also measured using various human C5 concentrations (40, 20, 10, 5, 2.5, 1.25 and 0.625 nM), and results are shown in FIG. 4 and Table 4. Collectively, these experiments showed that although ALXN1210 has more significant pH-dependent binding to human C5, the binding affinity of ALXN1210 to C5 at pH 7.4 is also the lowest of the three antibodies tested. While Eculizumab has higher affinity to human C5 at pH 7.4, it displays the least pH-dependent binding. Notably, Y32H/Q38R/L54H demonstrates significant pH-dependent C5 binding with 35.92% dissociation at pH 5.8 while maintaining high affinity of 3.72×10⁻¹⁰ M at pH 7.4.

TABLE 3
pH dependence of human C5 against antibodies

| | | Association/ | Dissociation | Dissociation |
|---|---|---|---|---|
| | | Dissociation | at 60 s | at 600 s |
| Sample | Antigen | pH | (%, n = 2) | (%, n = 2) |
| Y32H/Q38R/ | human | 7.4, 7.4 | 0.95 ± 0.32 | 5.65 ± 0.06 |
| L54H | C5 | | | |
| | human | 7.4, 5.8 | 5.02 ± 0.20 | 35.92 ± 0.47 |
| | C5 | | | |
| Eculizumab | human | 7.4, 7.4 | 0.28 ± 0.61 | 2.41 ± 0.11 |
| | C5 | | | |
| | human | 7.4, 5.8 | 0.73 ± 0.56 | 14.48 ± 0.72 |
| | C5 | | | |
| ALXN1210 | human | 7.4, 7.4 | 1.58 ± 0.24 | 10.56 ± 0.77 |
| | C5 | | | |
| | human | 7.4, 5.8 | 18.87 ± 0.75 | 83.07 ± 1.03 |
| | C5 | | | |

TABLE 4
Rate constants of human antigen against antibodies

| | | Asso- | | | |
|---|---|---|---|---|---|
| | | ciation/ | | | |
| | | Disso- | | | |
| | Anti- | ciation | | | |
| Sample | gen | pH | koff(1/s) | kon(1/Ms) | KD(M) |
| Y32H/Q38R/ | human | 7.4, 7.4 | 8.58 × 10⁻⁵ | 2.32 × 10⁵ | 3.71 × 10⁻¹⁰ |
| L54H | C5 | | | | |
| Eculizumab | human | 7.4, 7.4 | 1.69 × 10⁻⁵ | 2.10 × 10⁵ | 8.03 × 10⁻¹¹ |
| | C5 | | | | |
| ALXN1210 | human | 7.4, 7.4 | 1.52 × 10⁻⁵ | 1.26 × 10⁵ | 1.21 × 10⁻⁹ |
| | C5 | | | | |

3. pH Dependent Binding Assays with the Anti-C5-Factor H Fusion Antibodies

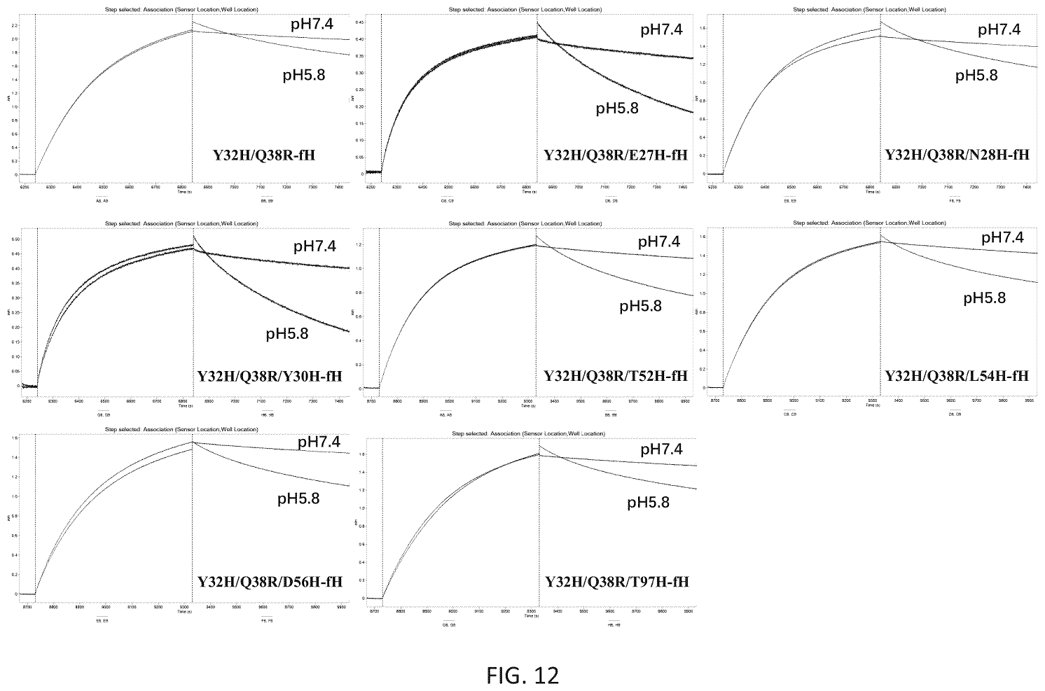

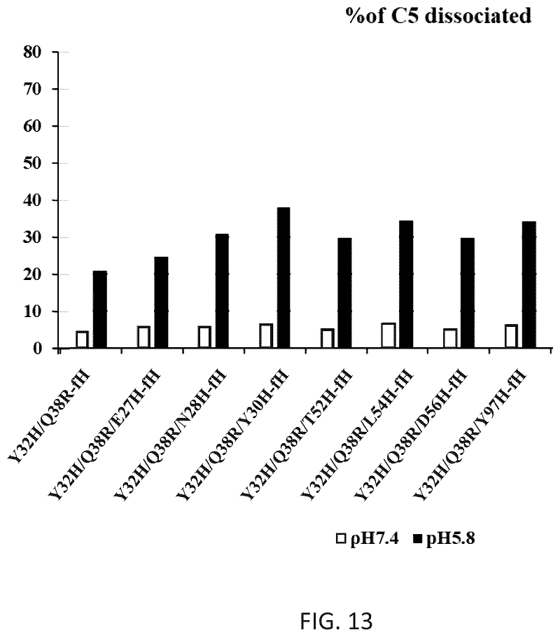

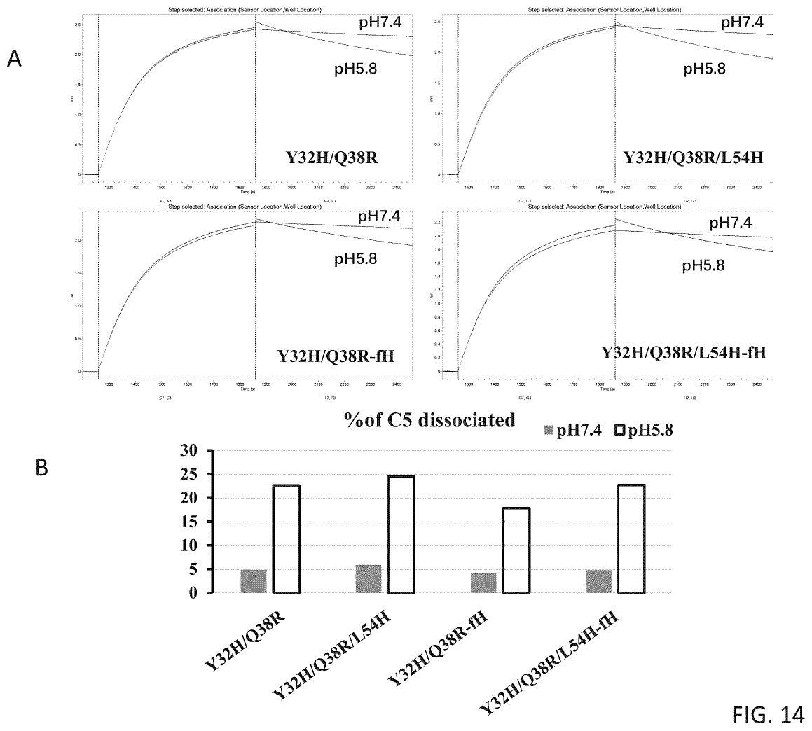

The respective anti-C5 mAb-factor H fusion proteins were tested for their pH-dependent C5 binding using similar methods as described above. As shown in FIGS. 12 and 13, all antibody fusion proteins showed similar pH-dependent C5 binding at pH 7.4 and pH 5.8 as was observed with the corresponding anti-C5 antibodies. In a direct comparison between two anti-C5 antibodies, Y32H/Q38R, Y32H/Q38R/L54H, and their corresponding factor H SCR1-5 fusion proteins, similar degrees of pH-dependent C5 binding at pH 7.4 and pH 5.8 were observed (FIG. 14).

### Example 3: In Vitro Functional Assays

### 1. Sheep Red Blood Cell Lysis Assay

A classical pathway complement-mediated sheep red blood cell lysis assay was used to assess the C5 inhibitory effect of all anti-C5 antibody mutants and their factor H fusion proteins.

Antibody-sensitized sheep RBCs (1×10⁷ cells/assay in PBS, Complement Technology Inc) were incubated at 37° C. for 20 min with 50% normal human serum (NHS, from Complement Technology Inc) in gelatin veronal buffer (GVB2+, Sigma total assay volume: 100 μl). NHS was pre-incubated with anti-C5 mAbs for 1 hr at 4° C. before addition into the sheep RBCs. Lysis reaction was stopped by addition of 40 mM EDTA in ice-cold PBS. The incubation mixtures were centrifuged for 5 min at 1500 rpm. The supernatant from each mixture was collected and measured for OD405 nm. Samples without NHS or with EDTA added were used as negative lysis controls, and a sample of sheep RBCs lysed completely with distilled water was used as a positive control (100% lysis) against which % lysis in other samples was normalized.

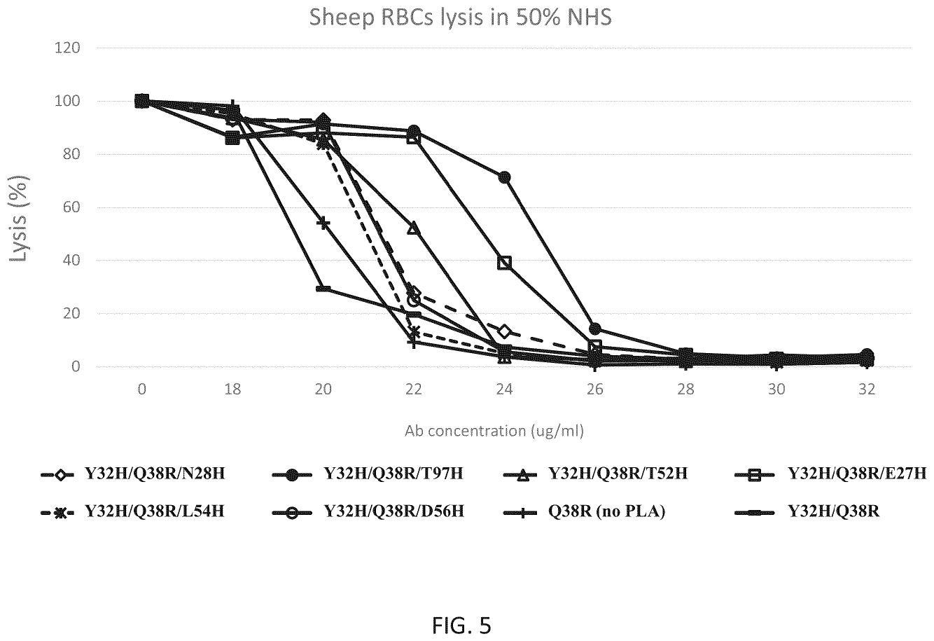

FIG. 5 shows the result of inhibition of RBC lysis by variants of anti-C5 antibodies at different antibody concentration levels. All anti-C5 antibodies protect against lysis at 18-26 μg/mL. Y32H/Q38R shows the most potent inhibition of sheep RBC lysis, with over 70% protection at 20 μg/mL. At 28 μg/mL, all anti-C5 antibodies were able to fully protect sheep RBCs from lysing. These results confirmed that the mutant anti-C5 antibody constructs maintain their functional activity while being capable of binding to C5 in a pH-dependent manner.

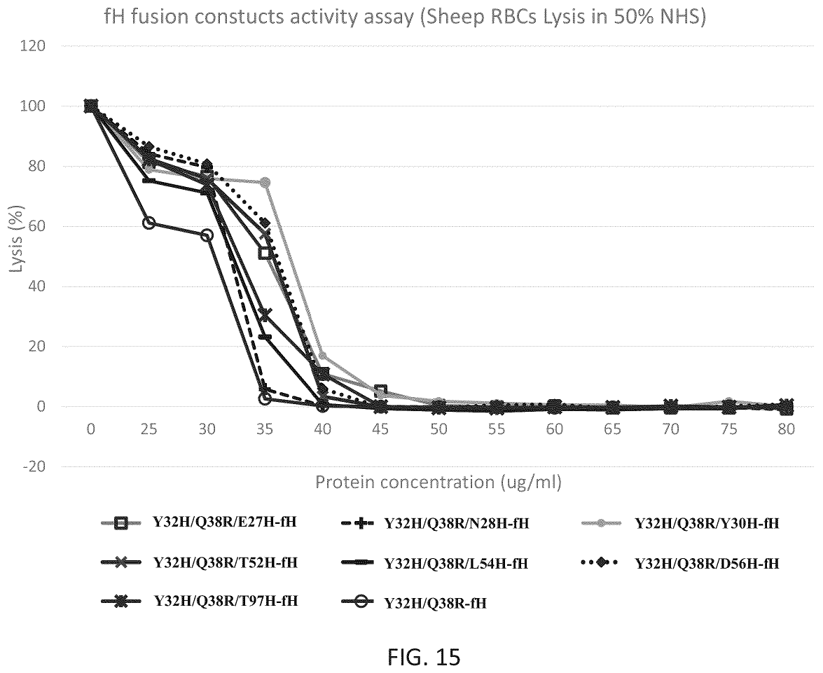

Similarly, FIG. 15 shows the result of inhibition of sheep RBC lysis by anti-C5 antibody-factor H fusion proteins. All anti-C5 antibody-factor H fusion proteins protect against sheep RBC lysis at 25-45 μg/mL. Y32H/Q38R/L54H-fH and Y32R/Q38R/N28H-fH show the most potent inhibition of sheep RBC lysis, with over 70% protection at 35 μg/mL. At 50 μg/mL, all anti-C5 antibody-factor H fusion proteins are able to fully protect sheep RBCs from lysing. These results confirmed that the mutant anti-C5 antibody factor H fusion protein constructs maintain their functional activity while being capable of binding to C5 in a pH-dependent manner.

### 2. Rabbit Red Blood Cell Lysis Assay

To assess the inhibitory activity on alternative pathway complement-mediated hemolysis by Y32H/Q38R and Y32H/Q38R-fH, a rabbit red blood cell lysis assay was performed, Rabbit RBCs (Rockland Immunochemicals Inc cat R403-0100) (1×10⁷ cells per assay sample prepared in PBS, Complement Technology Inc.) were incubated at 37° C. for 30 min with 25% normal human serum (NHS, from Complement Technology Inc.) in gelatin veronal buffer (GVB2+EGTA, Sigma; total assay volume: 100 μL). Before addition to the rabbit RBCs, NHS was pre-incubated with anti-C5 mAbs or anti-C5 mAb-fH SCR1-5 fusion proteins for 1 hr at 4° C. Lysis reaction was stopped by addition of 40 mM EDTA dissolved in ice-cold PBS. The incubation mixtures were centrifuged for 5 min at 1500 rpm. The supernatant was collected and measured for OD405 nm. Samples without NHS or with EDTA added were used as negative lysis controls, and a sample of rabbit RBCs lysed completely with distilled water was used as a positive control (100% lysis) against which % lysis in other samples was normalized.

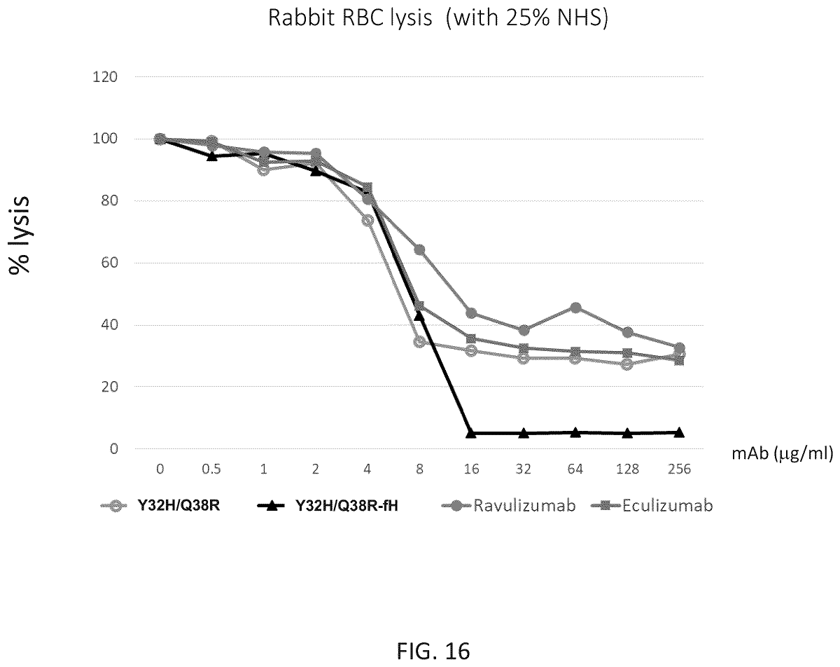

FIG. 16 shows that compared to the anti-C5 mAb, Y32H/Q38R, and benchmark anti-C5 antibodies Eculizumab and ALXN1210, Y32H/Q38R-fH is much more potent in inhibiting rabbit RBC lysis as it eliminated residual lysis that was still observed with the other three anti-C5 mAbs even at high concentrations. This may be explained by the fact that rabbit RBCs are exceedingly sensitive to C3 complement activation which can be regulated by the protein in the anti-C5 antibody-FH fusion protein.

### 3. FACs Analysis of C3 Deposition on Non-Lysed Rabbit RBC

Eculizumab and Ravulizumab have been used as a treatment for PNH, a rare hematologic disorder caused by the proliferation of a few hematopoietic stem cells that are defective of glycosylphosphatidylinositol (GPI) anchor protein synthesis. However, some PNH patients show sub-optimal response to Eculizumab and still require blood transfusion to alleviate anemia. This lack of complete response to Eculizumab has been linked to C3 opsonization and extravascular hemolysis.

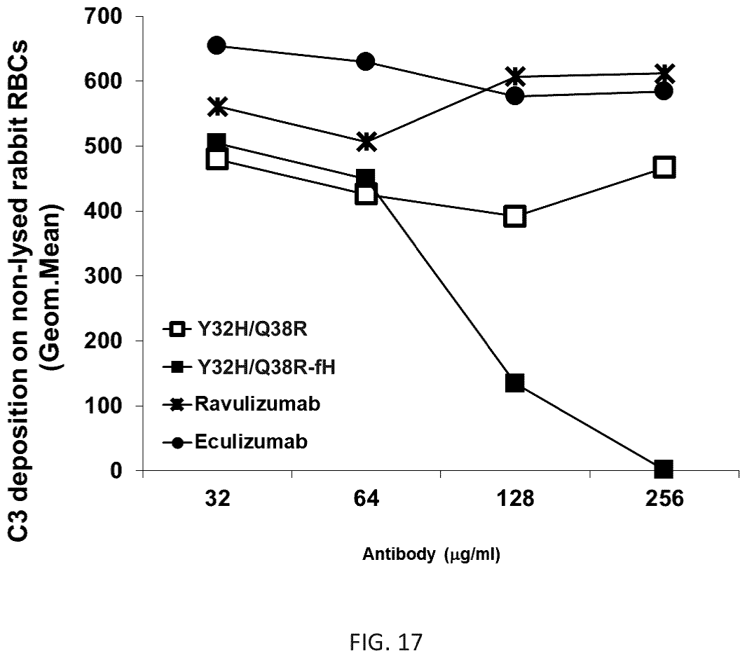

The effect of Y32H/Q38R, Y32H/Q38R-fH, and two benchmark anti-C5 antibodies Eculizumab and ALXN1210 on C3b opsonization was tested on non-lysed rabbit RBC after hemolytic reaction. Rabbit RBCs were washed twice in 20 mM EDTA/DPBS, resuspended in pH 7.4 assay buffer (GVB buffer, Sigma; 5 mM Mg²⁺, 20 mM EGTA). NHS (Complement Technology) was pre-incubated with antibody on ice for 60 min. Rabbit, RBC was added to serum/antibody mixture to 96-well plates, mix gently and incubate at 37° C. for 30 min. 100 μl of 20 mM EDTA was added to the mixture to stop the reaction. Plates were spun down at 1500 rpm for 5 min at 4° C. 100 μl supernatant from each well was transferred to a new 96 well plate and OD405 was measured on a microplate reader. Non-lysed RBCs were transferred into the 96-well plate and washed twice by FACS buffer. Cells were stained with 100 μg/mL of F(ab)₂ FITC anti-human C3 polyclonal antibody (MP biomedical; 1/200 dilution) on ice for 60 minutes. Cells were washed by FACS buffer (200□l) twice. C3 deposition on the cells was measured using a FACScalibur system and analyzed with FlowJo software. FIG. 17 depicts the result of FACS analysis of C3b deposition experiment on rabbit RBCs.

### 4. PNH Red Blood Cell Lysis Assay

The potency of C3 activation inhibition by Y32H/Q38R-fH was further investigated in a PNH red blood cell lysis assay using similar experimental procedure as described above. In brief, RBCs from whole blood of individual PNH patients were washed with DPBS and resuspended in an assay buffer (pH 6.4). Y32H/Q38R-fH or the anti-C5 antibodies were pre-incubated with acidified AB-NHS (normal human serum from AB blood type donors), then the mixture was added to the RBCs. The supernatant of RBC lysis was measured by a spectrometer at 405 nm.

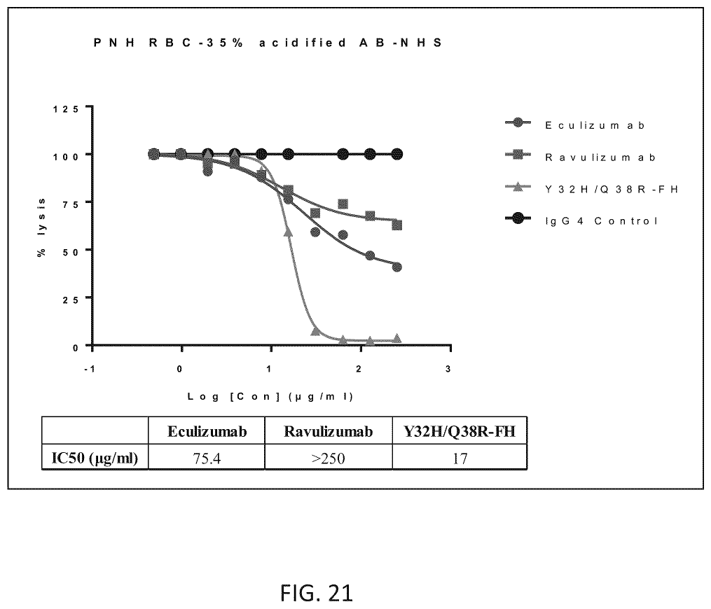

FIG. 21 shows that compared to the anti-C5 antibody Y32H/Q38R and benchmark anti-C5 antibodies Eculizumab and ALXN1210, Y32H/Q38R-fH is much more potent in inhibiting PNH RBC lysis by acidified human serum. These results highlight that the bi-functional Y32H/Q38R-fH, effectively inhibited C3 convertase to restrict C3b generation, thus inhibiting C3b deposition on protected PNH RBCs.

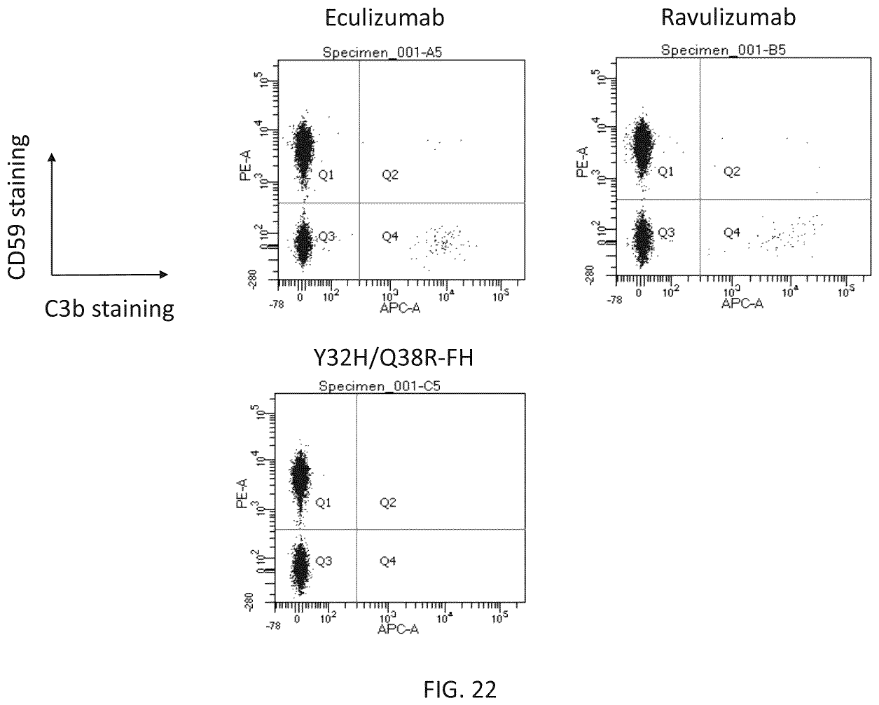

Similar experimental procedures described above for C3b deposition on non-lysed rabbit red blood cells can be applied to analysis of C3b deposition on non-lysed human PNH RBCs after ex vivo hemolytic assays using acidified human serum. FIG. 22 showed a FACS scatter plot of C3b and CD59 double staining on non-lysed. PNH RBCs from such an experiment. Dots in quadrant 4 represented C3b-positive PNH red blood cells.

The FACS results shown in FIGS. 17 and 22 demonstrated that Y32H/Q38R-fH effectively reduced C3b deposition in a dose-dependent manner on non-lysed rabbit RBC and human PNH RBCs, whereas Y32H/Q38R, Eculizumab or ALXN1210 treatment showed high level of C3b deposition on the non-lysed RBCs.

### Example 4: In Vivo Pharmacokinetics Evaluation

To test in vivo pharmacokinetics, C5 humanized mice on SCID/human FcRn transgenic background (human FcRn transgenic and mouse FcRn knockout) were generated. Sandwich ELBA was used for detection of human C5 in FcRn/SCID mice expressing human C5 after hydrodynamic injection of human C5 cDNA plasmid. 96-well plates were coated with an anti-human C5 antibody (Quidel, A217) at a final concentration of 2 μg/mL in bicarbonate buffer at 37° C. for 1 hr. Following washes with PBS containing 0.05% Tween-20, the plates were incubated with diluted plasma samples in blocking solution at RT for 1 hr. The plates were then washed and incubated with biotinylated anti-human C5 mAb 9G6 in blocking solution at RT for 1 hr, washed again and incubated with avidin or streptavidin conjugated to horseradish peroxidase (BD pharmigen) in blocking solution at RT for 1 hr. After final washing, the plates were developed with HRP substrate for 3 min. The reaction was stopped with 2N H2SO4 and the plate was read at 450 nm in a micro plate reader.

Human IgG4 in mice treated with the anti-C5 antibodies and anti-C5 antibody-fH fusion protein was detected using sandwich ELISA. 96-well plates were coated with an anti-human kappa light chain antibody (Antibody Solutions, AS75-P) at a final concentration of 2 μg/mL in bicarbonate buffer at 37° C. for 1 hr. Following three washes with PBS containing 0.05% Tween-20, the plates were incubated with diluted plasma samples in blocking solution at RT for 1 hr. After washing, the plates were incubated with anti-human IgG4 HRP (1:2000 dilution, Invitrogen, A10654) in blocking solution at RT for 1 hr. After washing, the plates were developed with HRP substrate for 3 min. The reaction was stopped with 2N H₂SO₄ and the plate was read at 450 nm in a micro plate reader.

Human IgG4-fH fusion in mice treated with anti-human C5 IgG4-fH1-5 fusion was also detected using sandwich ELISA. 96-well plates were coated with an anti-human kappa light chain antibody (Antibody Solutions, AS75-P) at a final concentration of 2 μg/mL in bicarbonate buffer at 37° C. for 1 hr. Following three washes with PBS containing 0.05% Tween-20, the plates were incubated with blocking solution at 37° C. for 1 hr. After washing, the plates were incubated with diluted plasma samples in blocking solution at RT for 1 hr. After washing, the plates were incubated with biotin-conjugated anti-human factor-H (1:100 dilution, Thermo Scientific, MA5-17735) in blocking solution at RT for 1 hr. After washing, the plates were incubated with streptavidin-HRP (1:1000 dilution, BD Biosciences, 554066) in blocking solution at RT for 1 hr. After washing, the plates were developed with HRP substrate (Thermo Scientific, 34029) for 3 min. The reaction was stopped with 2N H₂SO₄ and the plate was read at 450 nm in a micro plate reader

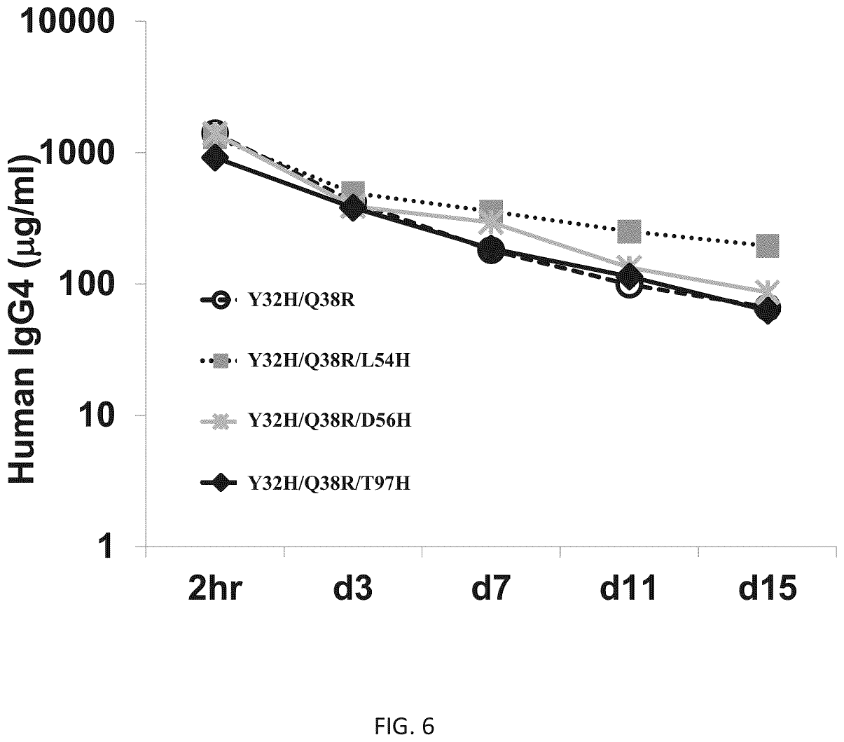

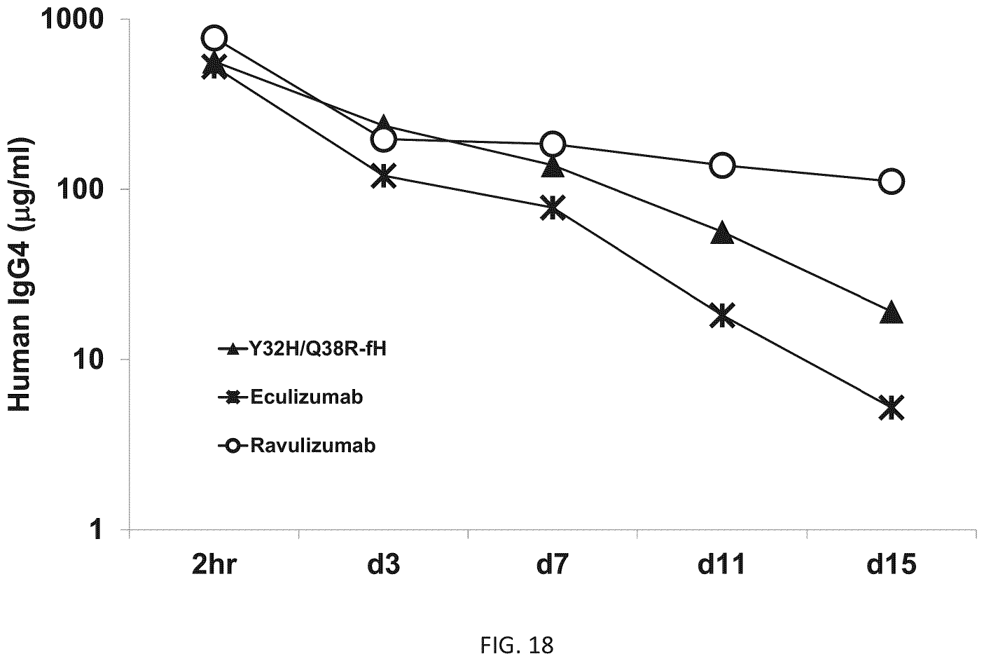

All histidine mutation-containing anti-C5 antibody constructs tested were shown to have good pharmacokinetics without compromising C5 blocking activity in vitro. FIG. 6 showed PK profile of the anti-C5 antibody Y32H/Q38R and 3 representative histidine mutants. The anti-C5 antibody-factor H fusion protein Y32H/Q38R-fH is shown to have superior PK than Eculizumab (FIG. 18).

### Example 5: Pharmacodynamics of Anti-C5 mAbs and Anti-C5 mAb-FH Fusion Proteins in FcRn/SCID Mice Expressing Human C5

To assess the pharmacodynamics of selected anti-C5 antibodies, a classical pathway complement-mediated chicken red blood cell assay was performed. To assess the pharmacodynamics of anti-C5 antibodies-EU fusion proteins, a rabbit red blood cell lysis test was performed. In both assays, a hybrid complement assay was used in which C5-depleted normal human serum (NHS) was mixed 1:1 with mouse serum collected at various time points after antibody treatment of C5-humanized SCID/human FcRn transgenic mice.

### 1. Rabbit Red Blood Cell Lysis Test Using Hybrid Human and Mouse Complement Systems

To evaluate the pharmacodynamics of Y32H/Q38R-fH in C5 humanized. SCID/human FcRn transgenic mice, rabbit RBC lysis test was performed using mouse plasma (as a source of free human C5) mixed with C5-depleted normal human serum. Mouse plasma was collected at different time points after drug treatment, and mouse C5 in the assay mixture was blocked with 2 anti-mouse C5 antibodies BB5.1 and 21A9 (final concentrations 400 ug/mL each). Mouse lepirudin plasma and C5-depleted human serum (Quidel #A501) samples were diluted to 10% in GVB EGTA buffer and mixed with 5 uL of rabbit RBC cells (5×10⁸/ml) in final volume of 50 uL and incubated at 37° C. for 30 min. Reactions were stopped with 100 uL of cold 10 mM EDTA in PBS. Cells were centrifuged at 1500 rpm for 5 min at 4° C. OD405 of the collected supernatant was measured.

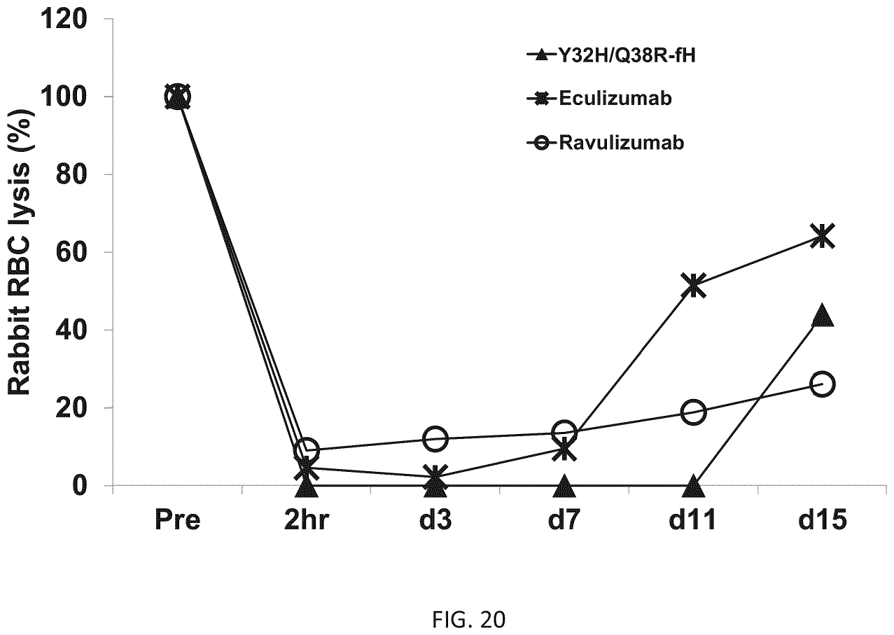

FIGS. 18 and 20 show that the Y32H/Q38R-fH fusion protein has longer half-life in C5 humanized mice SCID/human FcRn transgenic mice than the conventional benchmark anti-C5 mAb Eculizumab (FIG. 18), and it has better pharmacodynamics as measured in rabbit RBC lysis test than both Eculizumab and ALXN1210 (FIG. 20).

### 2. Chicken Red Blood Cell Lysis Assay Using Hybrid Human and Mouse Complement Systems

The pharmacodynamics of the anti-C5 mAbs Y32H/Q38R, Y32H/Q38R/L54H, Y32H/Q38R/D56H and Y32H/Q38R/T97H in C5-humanized SCID/human FcRn transgenic mice was also assessed by a chicken red blood cell lysis assay using hybrid human and mouse complement systems. Chicken RBCs (Rockland Immunochemicals Inc #R401-0050) were sensitized with anti-chicken RBC antibody (Rockland Immunochemicals Inc #103-4139) (150 ug/mL) for 30 min and washed two times with GVB buffer. Lepirudin-anticoagulated mouse plasma was pre-treated with two anti-mouse C5 antibodies BB5.1 and 21A9 (final concentrations 400 ug/mL each) to block murine C5 activity and then mixed (1:1) with C5-depleted human serum (Quidel #A501). The mixed samples were then diluted to 10% in GVB buffer (i.e., 5 μL to 50 μL final assay volume) and mixed with 5 μL of antibody-sensitized chicken RBC cells (2×10⁹/ml) in final volume of 50 μL and incubated at 37° C. for 30 min. Reactions were stopped with 100 μL of cold 10 mM EDTA in PBS. Cells were centrifuged at 1500 rpm for 5 min at 4° C. OD of the collected supernatant was measured at 405 nm.

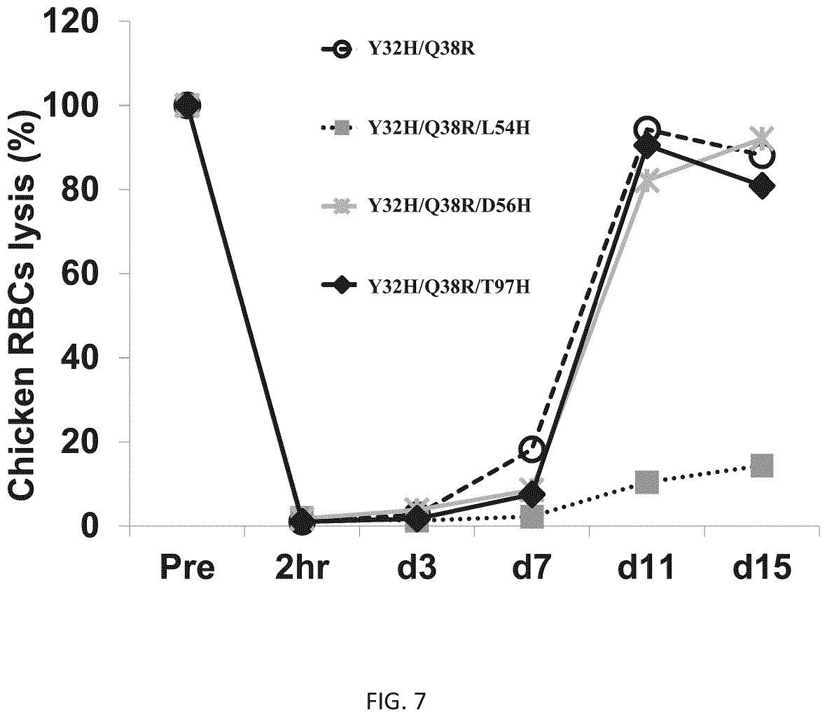

FIG. 7 shows the relative chicken RBC hemolytic activity of sera collected from C5-humanized SOD/human FcRn transgenic mice at different time points. The anti-C5 mAb Y32H/Q38R/L54H demonstrated superior pharmacodynamics activity compared with three other anti-C5 mAbs, Y32H/Q38R, Y32H/Q38R/D56H and Y32H/Q38R/T97H.

### Example 6: Cross-Species Evaluation

The cross-species reactivity of Y32H/Q38R/L54H in C5 binding was investigated in ELISA and sheep RBC lysis assays. ELISA wells were coated with recombinant C5 protein of relevant species (3 μg/ml in coating buffer, 100 μl/well, 4° C. overnight). After 3 washes with PBS containing 0.05% Tween 20, blocking with 1% BSA in PBS (200 μl/well, 25° C., 220 rpm, 1 hr) and washing again, serially diluted Y32H/Q38R/L54H or ALXN1210 was added into the wells and incubated at 37° C. Following the incubation, the wells were washed with buffer and bound Y32H/Q38R/L54H or ALXN1210 was detected by HRP conjugated anti-IgG4 Fc antibody. Sheep RBC lysis assay was performed as described in Example 3, using 5-20% cynomolgus monkey serum.

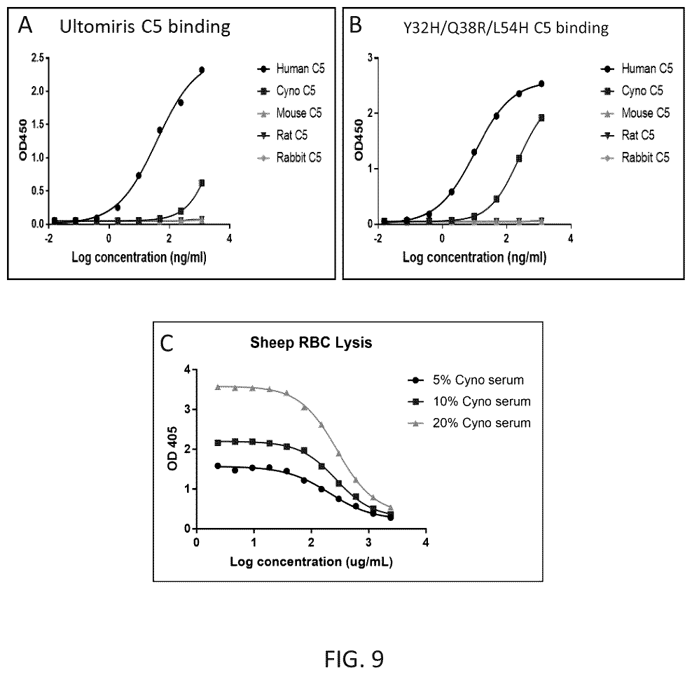

FIG. 9 shows that Y32H/Q38R/L54H displayed high affinity binding to human C5 with binding EC50 of 10.16 ng/ml. Y32H/Q38R/L54H showed no binding to recombinant mouse, rat and rabbit C5 protein in the same assay, but measurable binding to cynomolgus monkey C5 with EC50 of about 200 ng/ml. In the sheep RBC lysis assay, Y32H/Q38R/L54H potently inhibited human C5-mediated sheep RBC lysis with IC50 of 1.23 ug/ml (FIG. 5). Consistent with the binding data, Y32H/Q38R/L54H was also able to inhibit cynomolgus monkey C5-mediated sheep RBC lysis with 5-20% cynomolgus monkey serum at an antibody concentration of about 250 μg/mL. The cross-reactivity with cynomolgus C5 suggested that introduction of point mutations may cause significant structural changes in the antigen binding sites, inducing epitope shifting.

TABLE 5
Sequence Listings

| SEQ | | |
|---|---|---|
| ID | Notes | Amino Acid Sequences (mutations highlighted in yellow) |
| 1. | Anti-C5 mAb | QVQLVQSGAEVKKPGASVKVSCKASGYIFSNYWIQWVRQA |
| | VH no mutation | PGQGLEWMGEILPGSGSTEYTENFKDRVTMTRDTSTSTVYM |
| | | ELSSLRSEDTAVYYCARYFFGSSPNWYFDVWGQGTLVTVSS |
| 2. | Anti-C5 mAb | DIQMTQSPSSLSASVGDRVTITCGASENIYGALNWYQQKPGK |
| | VL no mutation | APKLLIYGATNLADGVPSRFSGSGSGTDFTLTISSLQPEDFAT |
| | | YYCQNVLNTPLTFGQGTKVEIK |
| 3. | H-CDR1 | ASGYIFSNHWIQ |
| | containing | |
| | Y32H | |
| 4. | H-CDR2 of | EILPGSGSTE |
| | anti-C5 mAb | |
| | VH no mutation | |
| 5. | H-CDR3 of | ARYFFGSSPNWYFDV |
| | anti-C5 mAb | |
| | VH no mutation | |
| 6. | L-CDR1 of | GASENIYGALN |
| | anti-C5 mAb | |
| | VL no mutation | |
| 7. | L-CDR2 of | YGATNLAD |
| | anti-C5 mAb | |
| | VL no mutation | |
| 8. | L-CDR3 of | QNVLNTPLT |
| | anti-C5 mAb | |
| | VL no mutation | |
| 9. | VH of | QVQLVQSGAEVKKPGASVKVSCKASGYIFSNHWIQWVRQA |
| | Y32H | PGQGLEWMGEILPGSGSTEYTENFKDRVTMTRDTSTSTVYM |
| | mutant | ELSSLRSEDTAVYYCARYFFGSSPNWYFDVWGQGTLVTVSS |
| 10 | VH of | QVQLVQSGAEVKKPGASVKVSCKASGYIFSNHWIQWVRQA |
| | Y32H- | PGQGLEWMGEILPGSGSTEYTENFKDRVTMTRDTSTSTVYM |
| | IgG4PLA-fH1-5 | ELSSLRSEDTAVYYCARYFFGSSPNWYFDVWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFLGGPSVFLFPPKP |
| | | KDTLMISRTPEVTCVVVDVSQEDPEVQFNWYVDGVEVHNA |
| | | KTKPREEQFNSTYRVVSVLTVLHQDWLNGKEYKCKVSNKG |
| | | LPSSIEKTISKAKGQPREPQVYTLPPSQEEMTKNQVSLTCLVK |
| | | GFYPSDIAVEWESNGQPENNYKTTPPVLDSDGSFFLYSRLTV |
| | | DKSRWQEGNVFSCSVLHEALHAHYTQKSLSLSLGKEDCNEL |
| | | PPRRNTEILTGSWSDQTYPEGTQAIYKCRPGYRSLGNIIMVCR |
| | | KGEWVALNPLRKCQKRPCGHPGDTPFGTFTLTGGNVFEYGV |
| | | KAVYTCNEGYQLLGEINYRECDTDGWTNDIPICEVVKCLPVT |
| | | APENGKIVSSAMEPDREYHFGQAVRFVCNSGYKIEGDEEMH |
| | | CSDDGFWSKEKPKCVEISCKSPDVINGSPISQKIIYKENERFQY |
| | | KCNMGYEYSERGDAVCTESGWRPLPSCEEKSCDNPYIPNGD |
| | | YSPLRIKHRTGDEITYQCRNGFYPATRGNTAKCTSTGWIPAP |
| | | RCTLKP |
| 11 | L-CDR1 | GASHNIYGALN |
| | containing | |
| | E27H | |
| 12 | L-CDR1 | GASEHIYGALN |
| | containing | |
| | N28H | |
| 13 | L-CDR1 | GASENIHGALN |
| | containing | |
| | Y30H | |
| 14 | L-CDR2 | YGAHNLAD |
| | containing | |
| | T52H | |
| 15 | L-CDR2 | YGATNHAD |
| | containing | |
| | L54H | |
| 16 | L-CDR2 | YGATNLAH |
| | containing | |
| | D56H | |
| 17 | L-CDR3 | QNVLNTPLH |
| | containing | |
| | T97H | |
| 18 | VL, Q38R | DIQMTQSPSSLSASVGDRVTITCGASENIYGALNWYQRKPGK |
| | | APKLLIYGATNLADGVPSRFSGSGSGTDFTLTISSLQPEDFAT |
| | | YYCQNVLNTPLTFGQGTKVEIK |
| 19 | VL, | DIQMTQSPSSLSASVGDRVTITCGASHNIYGALNWYQRKPG |
| | Q38R/E27H | KAPKLLIYGATNLADGVPSRFSGSGSGTDFTLTISSLQPEDFA |
| | | TYYCQNVLNTPLTFGQGTK VEIK |
| 20 | VL, | DIQMTQSPSSLSASVGDRVTITCGASEHIYGALNWYQRKPGK |
| | Q38R/N28H | APKLLIYGATNLADGVPSRFSGSGSGTDFTLTISSLQPEDFAT |
| | | YYCQNVLNTPLTFGQGTKVEIK |
| 21 | VL, | DIQMTQSPSSLSASVGDRVTITCGASENIHGALNWYQRKPGK |
| | Q38R/Y30H | APKLLIYGATNLADGVPSRFSGSGSGTDFTLTISSLQPEDFAT |
| | | YYCQNVLNTPLTFGQGTKVEIK |
| 22 | VL, | DIQMTQSPSSLSASVGDRVTITCGASENIYGALNWYQRKPGK |
| | Q38R/T52H | APKLLIYGAHNLADGVPSRFSGSGSGTDFTLTISSLQPEDFAT |
| | | YYCQNVLNTPLTFGQGTKVEIK |
| 23 | VL, | DIQMTQSPSSLSASVGDRVTITCGASENIYGALNWYQRKPGK |
| | Q38R/L54H | APKLLIYGATNHADGVPSRFSGSGSGTDFTLTISSLQPEDFAT |
| | | YYCQNVLNTPLTFGQGTKVEIK |
| 24 | VL, | DIQMTQSPSSLSASVGDRVTITCGASENIYGALNWYQRKPGK |
| | Q38R/D56H | APKLLIYGATNLAHGVPSRFSGSGSGTDFTLTISSLQPEDFAT |
| | | YYCQNVLNTPLTFGQGTKVEIK |
| 25 | VL, | DIQMTQSPSSLSASVGDRVTITCGASENIYGALNWYQRKPGK |
| | Q38R/T97H | APKLLIYGATNLADGVPSRFSGSGSGTDFTLTISSLQPEDFAT |
| | | YYCQNVLNTPLHFGQGTKVEIK |
| 26 | WT IgG4 Fc | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFLGGPSVFLFPPKP |
| | | KDTLMISRTPEVTCVVVDVSQEDPEVQFNWYVDGVEVHNA |
| | | KTKPREEQFNSTYRVVSVLTVLHQDWLNGKEYKCKVSNKG |
| | | LPSSIEKTISKAKGQPREPQVYTLPPSQEEMTKNQVSLTCLVK |
| | | GFYPSDIAVEWESNGQPENNYKTTPPVLDSDGSFFLYSRLTV |
| | | DKSRWQEGNVFSCSVMHEALHNHYTQKSLSLSLGK |
| 27 | IgG4 Fc with | ASTKGPSVFPLAPCSRSTSESTAALGCLVKDYFPEPVTVSWN |
| | PLA mutations | SGALTSGVHTFPAVLQSSGLYSLSSVVTVPSSSLGTKTYTCN |
| | | VDHKPSNTKVDKRVESKYGPPCPPCPAPEFLGGPSVFLFPPKP |
| | | KDTLMISRTPEVTCVVVDVSQEDPEVQFNWYVDGVEVHNA |
| | | KTKPREEQFNSTYRVVSVLTVLHQDWLNGKEYKCKVSNKG |
| | | LPSSIEKTISKAKGQPREPQVYTLPPSQEEMTKNQVSLTCLVK |
| | | GFYPSDIAVEWESNGQPENNYKTTPPVLDSDGSFFLYSRLTV |
| | | DKSRWQEGNVFSCSVLHEALHAHYTQKSLSLSLGK |
| 28 | Y32H heavy | QVQLVQSGAEVKKPGASVKVSCKASGYIFSNHWIQWVRQA |
| | chain with PLA | PGQGLEWMGEILPGSGSTEYTENFKDRVTMTRDTSTSTVYM |
| | mutation | ELSSLRSEDTAVYYCARYFFGSSPNWYFDVWGQGTLVTVSS |
| | | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFLGGPSVFLFPPKP |
| | | KDTLMISRTPEVTCVVVDVSQEDPEVQFNWYVDGVEVHNA |
| | | KTKPREEQFNSTYRVVSVLTVLHQDWLNGKEYKCKVSNKG |
| | | LPSSIEKTISKAKGQPREPQVYTLPPSQEEMTKNQVSLTCLVK |
| | | GFYPSDIAVEWESNGQPENNYKTTPPVLDSDGSFFLYSRLTV |
| | | DKSRWQEGNVFSCSVLHEALHAHYTQKSLSLSLGK |
| 29 | Y32H heavy | QVQLVQSGAEVKKPGASVKVSCKASGYIFSNHWIQWVRQA |
| | chain with PLA | PGQGLEWMGEILPGSGSTEYTENFKDRVTMTRDTSTSTVYM |
| | mutation factor | ELSSLRSEDTAVYYCARYFFGSSPNWYFDVWGQGTLVTVSS |
| | H fusion | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRVESKYGPPCPPCPAPEFLGGPSVFLFPPKP |
| | | KDTLMISRTPEVTCVVVDVSQEDPEVQFNWYVDGVEVHNA |
| | | KTKPREEQFNSTYRVVSVLTVLHQDWLNGKEYKCKVSNKG |
| | | LPSSIEKTISKAKGQPREPQVYTLPPSQEEMTKNQVSLTCLVK |
| | | GFYPSDIAVEWESNGQPENNYKTTPPVLDSDGSFFLYSRLTV |
| | | DKSRWQEGNVFSCSVLHEALHAHYTQKSLSLSLGKEDCNEL |
| | | PPRRNTEILTGSWSDQTYPEGTQAIYKCRPGYRSLGNIIMVCR |
| | | KGEWVALNPLRKCQKRPCGHPGDTPFGTFTLTGGNVFEYGV |
| | | KAVYTCNEGYQLLGEINYRECDTDGWTNDIPICEVVKCLPVT |
| | | APENGKIVSSAMEPDREYHFGQAVRFVCNSGYKIEGDEEMH |
| | | CSDDGFWSKEKPKCVEISCKSPDVINGSPISQKIIYKENERFQY |
| | | KCNMGYEYSERGDAVCTESGWRPLPSCEEKSCDNPYIPNGD |
| | | YSPLRIKHRTGDEITYQCRNGFYPATRGNTAKCTSTGWIPAP |
| | | RCTLKP |
| 30 | C5 (human) | MGLLGILCFLIFLGKTWGQEQTYVISAPKIFRVGASENIVIQV |
| | | YGYTEAFDATISIKSYPDKKFSYSSGHVHLSSENKFQNSAILTI |
| | | QPKQLPGGQNPVSYVYLEVVSKHFSKSKRMPITYDNGFLFIH |
| | | TDKPVYTPDQSVKVRVYSLNDDLKPAKRETVLTFIDPEGSEV |
| | | DMVEEIDHIGIISFPDFKIPSNPRYGMWTIKAKYKEDFSTTGT |
| | | AYFEVKEYVLPHFSVSIEPEYNFIGYKNFKNFEITIKARYFYN |
| | | KVVTEADVYITFGIREDLKDDQKEMMQTAMQNTMLINGIAQ |
| | | VTFDSETAVKELSYYSLEDLNNKYLYIAVTVIESTGGFSEEAE |
| | | IPGIKYVLSPYKLNLVATPLFLKPGIPYPIKVQVKDSLDQLVG |
| | | GVPVTLNAQTIDVNQETSDLDPSKSVTRVDDGVASFVLNLPS |
| | | GVTVLEFNVKTDAPDLPEENQAREGYRAIAYSSLSQSYLYID |
| | | WTDNHKALLVGEHLNIIVTPKSPYIDKITHYNYLILSKGKIIHF |
| | | GTREKFSDASYQSINIPVTQNMVPSSRLLVYYIVTGEQTAELV |
| | | SDSVWLNIEEKCGNQLQVHLSPDADAYSPGQTVSLNMATG |
| | | MDSWVALAAVDSAVYGVQRGAKKPLERVFQFLEKSDLGCG |
| | | AGGGLNNANVFHLAGLTFLTNANADDSQENDEPCKEILRPR |
| | | RTLQKKIEEIAAKYKHSVVKKCCYDGACVNNDETCEQRAAR |
| | | ISLGPRCIKAFTECCVVASQLRANISHKDMQLGRLHMKTLLP |
| | | VSKPEIRSYFPESWLWEVHLVPRRKQLQFALPDSLTTWEIQG |
| | | VGISNTGICVADTVKAKVFKDVFLEMNIPYSVVRGEQIQLKG |
| | | TVYNYRTSGMQFCVKMSAVEGICTSESPVIDHQGTKSSKCV |
| | | RQKVEGSSSHLVTFTVLPLEIGLHNINFSLETWFGKEILVKTL |
| | | RVVPEGVKRESYSGVTLDPRGIYGTISRRKEFPYRIPLDLVPK |
| | | TEIKRILSVKGLLVGEILSAVLSQEGINILTHLPKGSAEAELMS |
| | | VVPVFYVFHYLETGNHWNIFHSDPLIEKQKLKKKLKEGMLSI |
| | | MSYRNADYSYSVWKGGSASTWLTAFALRVLGQVNKYVEQ |
| | | NONSICNSLLWLVENYQLDNGSFKENSQYQPIKLQGTLPVEA |
| | | RENSLYLTAFTVIGIRKAFDICPLVKIDTALIKADNFLLENTLP |
| | | AQSTFTLAISAYALSLGDKTHPQFRSIVSALKREALVKGNPPI |
| | | YRFWKDNLQHKDSSVPNTGTARMVETTAYALLTSLNLKDIN |
| | | YVNPVIKWLSEEQRYGGGFYSTQDTINAIEGLTEYSLLVKQL |
| | | RLSMDIDVSYKHKGALHNYKMTDKNFLGRPVEVLLNDDLIV |
| | | STGFGSGLATVHVTTVVHKTSTSEEVCSFYLKIDTQDIEASHY |
| | | RGYGNSDYKRIVACASYKPSREESSSGSSHAVMDISLPTGISA |
| | | NEEDLKALVEGVDQLFTDYQIKDGHVILQLNSIPSSDFLCVRF |
| | | RIFELFEVGFLSPATFTVYEYHRPDKQCTMFYSTSNIKIQKVC |
| | | EGAACKCVEADCGQMQEELDLTISAETRKQTACKPEIAYAY |
| | | KVSITSITVENVFVKYKATLLDIYKTGEAVAEKDSEITFIKKV |
| | | TCTNAELVKGRQYLIMGKEALQIKYNFSFRYIYPLDSLTWIE |
| | | YWPRDTTCSSCQAFLANLDEFAEDIFLNGC |
| 31 | Signal peptide | MKWSWVILFLLSVTAGVHS |
| 32 | Signal peptide | MEAPAQLLFLLLLWLPDTTG |
| 33 | Anti-C5 mAb | caggttcagctggtgcagtctggagctgaggtgaagaagcctggggcctcagtgaaggtttcct |
| | VH no mutation | gcaaggcttctggctacatattcagtaactactggatacagtgggtgcgacaggcccctggacaa |
| | | ggccttgagtggatgggtgagattttacctggaagtggttctactgagtacactgagaacttcaag |
| | | gacagggtcaccatgactagggatacatccacgagcacagtctacatggagctcagcagcctg |
| | | agatcagaggacacggccgtctattactgtgcaagatatttcttcggtagtagccccaactggtac |
| | | ttcgatgtctggggccaggggaccctggtcaccgtctcctca |
| 34 | Anti-C5 mAb | gacatccagatgactcagtctccatcttcactgtctgcatctgtgggagaccgggtcaccatcaca |
| | VL no mutation | tgtggagcaagtgagaatatttacggtgctttaaattggtatcagcagaaacctggaaaagcacct |
| | | aagctcctgatctatggtgcaaccaacttggcagatggcgtcccttcgaggttcagtggcagtgg |
| | | atctggtaccgatttcacgctcactatcagtagcctgcagcctgaggatttcgcaacgtattactg |
| | | tcaaaatgtgttaaatactcctctcacgttcggtcaggggaccaaggtggagatcaaa |
| 35 | H-CDR1 | ggctacatattcagtaaccattggatacag |
| | containing | |
| | Y32H | |
| 36 | H-CDR2 of | gagattttacctggaagtggttctactgag |
| | anti-C5 mAb | |
| | VH no mutation | |
| 37 | H-CDR3 of | gcaagatatttcttcggtagtagccccaactggtacttcgatgtc |
| | anti-C5 mAb | |
| | VH no mutation | |
| 38 | L-CDR1 of | ggagcaagtgagaatatttacggtgctttaaat |
| | anti-C5 mAb | |
| | VL no mutation | |
| 39 | L-CDR2 of | tatggtgcaaccaacttggcagat |
| | anti-C5 mAb | |
| | VL no mutation | |
| 40 | L-CDR3 of | caaaatgtgttaaatactcctctcacg |
| | anti-C5 mAb | |
| | VL no mutation | |
| 41 | VH of | caggttcagctggtgcagtctggagctgaggtgaagaagcctggggcctcagtgaaggtttcct |
| | Y32H | gcaaggcttctggctacatattcagtaaccattggatacagtgggtgcgacaggcccctggacaa |
| | mutant | ggccttgagtggatgggtgagattttacctggaagtggttctactgagtacactgagaacttcaag |
| | | gacagggtcaccatgactagggatacatccacgagcacagtctacatggagctcagcagcctg |
| | | agatcagaggacacggccgtctattactgtgcaagatatttcttcggtagtagccccaactggtac |
| | | ttcgatgtctggggccaggggaccctggtcaccgtctcctca |
| 42 | VH of | caggttcagctggtgcagtctggagctgaggtgaagaagcctggggcctcagtgaaggtttcct |
| | Y32H- | gcaaggcttctggctacatattcagtaaccattggatacagtgggtgcgacaggcccctggacaa |
| | IgG4PLA-fH1-5 | ggccttgagtggatgggtgagattttacctggaagtggttctactgagtacactgagaacttcaag |
| | | gacagggtcaccatgactagggatacatccacgagcacagtctacatggagctcagcagcctg |
| | | agatcagaggacacggccgtctattactgtgcaagatatttcttcggtagtagccccaactggtac |
| | | ttcgatgtctggggccaggggaccctggtcaccgtctcctcagctagcaccaagggcccatcgg |
| | | tcttccccctggcgccctgctccaggagcacctccgagagcacagccgccctgggctgcctggt |
| | | caaggactacttccccgaaccggtgacggtgtcgtggaactcaggcgccctgaccagcggcgt |
| | | gcacaccttcccggctgtcctacagtcctcaggactctactccctcagcagcgtggtgaccgtgc |
| | | cctccagcagcttgggcacgaagacctacacctgcaacgtagatcacaagcccagcaacacca |
| | | aggtggacaagagagttgagtccaaatatggtcccccatgcccaccatgcccagcacctgagtt |
| | | cctggggggaccatcagtcttcctgttccccccaaaacccaaggacactctcatgatctcccgga |
| | | cccctgaggtcacgtgcgtggtggtggacgtgagccaggaagaccccgaggtccagttcaact |
| | | ggtacgtggatggcgtggaggtgcataatgccaagacaaagccgcgggaggagcagttcaac |
| | | agcacgtaccgtgtggtcagcgtcctcaccgtcctgcaccaggactggctgaacggcaaggag |
| | | tacaagtgcaaggtctccaacaaaggcctcccgtcctccatcgagaaaaccatctccaaagcca |
| | | aagggcagccccgagagccacaggtgtacaccttgccaccctcccaggaggagatgacaaaa |
| | | aatcaggtaagtctcacatgcctggtcaaaggtttttacccatccgacattgctgtagagtgggaa |
| | | tccaacggtcagcccgagaacaactacaagacaactcccccagtcttggactcagatggctcctt |
| | | cttcttgtatagtagactgacagttgataaaagccgctggcaagaaggcaatgtctttagctgtag |
| | | tgtcctgcatgaagcacttcacgcccattatacacaaaaatccctcagccttagcctagggaagga |
| | | ggattgtaatgaactgcctccaagaaggaacacagaaattttgaccgggtcctggtccgatcaaa |
| | | cctaccctgaaggaactcaagctatatacaagtgcagacctggttatagatcactgggcaacatta |
| | | tcatggtatgtcgcaaaggagaatgggtggctctgaatcccctcagaaaatgccagaaacggcc |
| | | atgtggacaccccggcgataccccattcgggacatttaccttgactggaggcaatgtattcgagta |
| | | tggcgtgaaagctgtctatacctgtaacgaaggttaccaattgttgggagaaataaattacagaga |
| | | atgtgataccgatggatggaccaacgatattcccatatgcgaggttgttaagtgcttgcctgtcac |
| | | tgcaccagaaaacgggaaaatcgtatctagcgcaatggagccagaccgcgaataccatttcggg |
| | | caggcagtgaggtttgtttgcaattccggttataagatagaaggggatgaggagatgcactgtag |
| | | cgacgatggtttttggtccaaggaaaagcccaagtgcgtcgaaataagttgcaagtcacctgacg |
| | | tcataaacgggagccccatatcccaaaagataatttacaaggagaacgaacgatttcaatataagt |
| | | gtaatatggggtatgaatactccgagagaggtgatgccgtctgtaccgaaagtggatggcgacc |
| | | actcccctcatgcgaagagaagtcctgtgataatccatacatccctaatggtgattattcccccct |
| | | tcgaataaagcatcggacaggagacgagatcacatatcagtgtcgcaacggattctatccagccac |
| | | cagaggcaacactgcaaagtgtacatctacaggatggatacctgccccacgatgtaccttgaag |
| | | ccctga |
| 43 | L-CDR1 | ggagcaagtcataatatttacggtgctttaaat |
| | containing | |
| | E27H | |
| 44 | L-CDR1 | ggagcaagtgagcatatttacggtgctttaaat |
| | containing | |
| | N28H | |
| 45 | L-CDR1 | ggagcaagtgagaatattcacggtgctttaaat |
| | containing | |
| | Y30H | |
| 46 | L-CDR2 | tatggtgcacacaacttggcagat |
| | containing | |
| | T52H | |
| 47 | L-CDR2 | tatggtgcaaccaaccatgcagat |
| | containing | |
| | L54H | |
| 48 | L-CDR2 | tatggtgcaaccaacttggcacat |
| | containing | |
| | D56H | |
| 49 | L-CDR3 | caaaatgtgttaaatactcctctccac |
| | containg T97H | |
| 50 | Kappa light | gacatccagatgactcagtctccatcttcactgtctgcatctgtgggagaccgggtcaccatcaca |
| | chain, Q38R | tgtggagcaagtgagaatatttacggtgctttaaattggtatcagcggaaacctggaaaagcacct |
| | | aagctcctgatctatggtgcaaccaacttggcagatggcgtcccttcgaggttcagtggcagtgg |
| | | atctggtaccgatttcacgctcactatcagtagcctgcagcctgaggatttcgcaacgtattactgt |
| | | caaaatgtgttaaatactcctctcacgttcggtcaggggaccaaggtggagatcaaa |
| 51 | Kappa light | gacatccagatgactcagtctccatcttcactgtctgcatctgtgggagaccgggtcaccatcaca |
| | chain, | tgtggagcaagtcataatatttacggtgctttaaattggtatcagcggaaacctggaaaagcacct |
| | Q38R/E27H | aagctcctgatctatggtgcaaccaacttggcagatggcgtcccttcgaggttcagtggcagtgg |
| | | atctggtaccgatttcacgctcactatcagtagcctgcagcctgaggatttcgcaacgtattactgt |
| | | caaaatgtgttaaatactcctctcacgttcggtcaggggaccaaggtggagatcaaa |
| 52 | Kappa light | gacatccagatgactcagtctccatcttcactgtctgcatctgtgggagaccgggtcaccatcaca |
| | chain, | tgtggagcaagtgagcatatttacggtgctttaaattggtatcagcggaaacctggaaaagcacct |
| | Q38R/N28H | aagctcctgatctatggtgcaaccaacttggcagatggcgtcccttcgaggttcagtggcagtgg |
| | | atctggtaccgatttcacgctcactatcagtagcctgcagcctgaggatttcgcaacgtattactgt |
| | | caaaatgtgttaaatactcctctcacgttcggtcaggggaccaaggggagatcaaa |
| 53 | Kappa light | gacatccagatgactcagtctccatcttcactgtctgcatctgtgggagaccgggtcaccatcaca |
| | chain, | tgtggagcaagtgagaatattcacggtgctttaaattggtatcagcggaaacctggaaaagcacc |
| | Q38R/Y30H | taagctcctgatctatggtgcaaccaacttggcagatggcgtcccttcgaggttcagtggcagtgg |
| | | atctggtaccgatttcacgctcactatcagtagcctgcagcctgaggatttcgcaacgtattactgt |
| | | caaaatgtgttaaatactcctctcacgttcggtcaggggaccaaggtggagatcaaa |
| 54 | Kappa light | gacatccagatgactcagtctccatcttcactgtctgcatctgtgggagaccgggtcaccatcaca |
| | chain, | tgtggagcaagtgagaatatttacggtgctttaaattggtatcagcggaaacctggaaaagcacct |
| | Q38R/T52H | aagctcctgatctatggtgcacacaacttggcagatggcgtcccttcgaggttcagtggcagtgg |
| | | atctggtaccgatttcacgctcactatcagtagcctgcagcctgaggatttcgcaacgtattactgt |
| | | caaaatgtgttaaatactcctctcacgttcggtcaggggaccaaggtggagatcaaa |
| 55 | Kappa light | gacatccagatgactcagtctccatcttcactgtctgcatctgtgggagaccgggtcaccatcaca |
| | chain, | tgtggagcaagtgagaatatttacggtgctttaaattggtatcagcggaaacctggaaaagcacct |
| | Q38R/L54H | aagctcctgatctatggtgcaaccaaccatgcagatggcgtcccttcgaggttcagtggcagtgg |
| | | atctggtaccgatttcacgctcactatcagtagcctgcagcctgaggatttcgcaacgtattactgt |
| | | caaaatgtgttaaatactcctctcacgttcggtcaggggaccaaggtggagatcaaa |
| 56 | Kappa light | gacatccagatgactcagtctccatcttcactgtctgcatctgtgggagaccgggtcaccatcaca |
| | chain, | tgtggagcaagtgagaatatttacggtgctttaaattggtatcagcggaaacctggaaaagcacct |
| | Q38R/D56H | aagctcctgatctatggtgcaaccaacttggcacatggcgtcccttcgaggttcagtggcagtgg |
| | | atctggtaccgatttcacgctcactatcagtagcctgcagcctgaggatttcgcaacgtattactgt |
| | | caaaatgtgttaaatactcctctcacgttcggtcaggggaccaaggtggagatcaaa |
| 57 | Kappa light | gacatccagatgactcagtctccatcttcactgtctgcatctgtgggagaccgggtcaccatcaca |
| | chain, | tgtggagcaagtgagaatatttacggtgctttaaattggtatcagcggaaacctggaaaagcacct |
| | Q38R/T97H | aagctcctgatctatggtgcaaccaacttggcagatggcgtcccttcgaggttcagtggcagtgg |
| | | atctggtaccgatttcacgctcactatcagtagcctgcagcctgaggatttcgcaacgtattactgt |
| | | caaaatgtgttaaatactcctctccacttcggtcaggggaccaaggtggagatcaaa |
| 58 | IgG4 Fc with | gctagcaccaagggcccatcggtcttccccctggcgccctgctccaggagcacctccgagagc |
| | PLA mutations | acagccgccctgggctgcctggtcaaggactacttccccgaaccggtgacggtgtcgtggaact |
| | | caggcgccctgaccagcggcgtgcacaccttcccggctgtcctacagtcctcaggactctactc |
| | | cctcagcagcgtggtgaccgtgccctccagcagcttgggcacgaagacctacacctgcaacgt |
| | | agatcacaagcccagcaacaccaaggtggacaagagagttgagtccaaatatggtcccccatg |
| | | cccaccatgcccagcacctgagttcctggggggaccatcagtcttcctgttccccccaaaaccca |
| | | aggacactctcatgatctcccggacccctgaggtcacgtgcgtggtggtggacgtgagccagg |
| | | aagaccccgaggtccagttcaactggtacgtggatggcgtggaggtgcataatgccaagacaa |
| | | agccgcgggaggagcagttcaacagcacgtaccgtgtggtcagcgtcctcaccgtcctgcacc |
| | | aggactggctgaacggcaaggagtacaagtgcaaggtctccaacaaaggcctcccgtcctcca |
| | | tcgagaaaaccatctccaaagccaaagggcagccccgagagccacaggtgtacaccctgccc |
| | | ccatcccaggaggagatgaccaagaaccaggtcagcctgacctgcctggtcaaaggcttctac |
| | | cccagcgacatcgccgtggagtgggagagcaatgggcagccggagaacaactacaagacca |
| | | cgcctcccgtgctggactccgacggctccttcttcctctacagcaggctcaccgtggacaagagc |
| | | aggtggcaggaggggaatgtcttctcatgctccgtgctgcatgaggctctgcacgcccactaca |
| | | cacagaagagcctctccctgtctctgggtaaa |
| 59 | Y32H heavy | caggttcagctggtgcagtctggagctgaggtgaagaagcctggggcctcagtgaaggtttcct |
| | chain with PLA | gcaaggcttctggctacatattcagtaaccattggatacagtgggtgcgacaggcccctggacaa |
| | mutation | ggccttgagtggatgggtgagattttacctggaagtggttctactgagtacactgagaacttcaag |
| | | gacagggtcaccatgactagggatacatccacgagcacagtctacatggagctcagcagcctg |
| | | agatcagaggacacggccgtctattactgtgcaagatatttcttcggtagtagccccaactggtac |
| | | ttcgatgtctggggccaggggaccctggtcaccgtctcctcagctagcaccaagggcccatcgg |
| | | tcttccccctggcgccctgctccaggagcacctccgagagcacagccgccctgggctgcctggt |
| | | caaggactacttccccgaaccggtgacggtgtcgtggaactcaggcgccctgaccagcggcgt |
| | | gcacaccttcccggctgtcctacagtcctcaggactctactecctcagcagcgtggtgaccgtgc |
| | | cctccagcagcttgggcacgaagacctacacctgcaacgtagatcacaagcccagcaacacca |
| | | aggtggacaagagagttgagtccaaatatggtcccccatgcccaccatgcccagcacctgagtt |
| | | cctggggggaccatcagtcttcctgttccccccaaaacccaaggacactctcatgatctcccgga |
| | | cccctgaggtcacgtgcgtggtggtggacgtgagccaggaagaccccgaggtccagttcaact |
| | | ggtacgtggatggcgtggaggtgcataatgccaagacaaagccgcgggaggagcagttcaac |
| | | agcacgtaccgtgtggtcagcgtcctcaccgtcctgcaccaggactggctgaacggcaaggag |
| | | tacaagtgcaaggtctccaacaaaggcctcccgtcctccatcgagaaaaccatctccaaagcca |
| | | aagggcagccccgagagccacaggtgtacaccctgcccccatcccaggaggagatgaccaa |
| | | gaaccaggtcagcctgacctgcctggtcaaaggcttctaccccagcgacatcgccgtggagtg |
| | | ggagagcaatgggcagccggagaacaactacaagaccacgcctcccgtgctggactccgacg |
| | | gctccttcttcctctacagcaggctcaccgtggacaagagcaggtggcaggaggggaatgtctt |
| | | ctcatgctccgtgctgcatgaggctctgcacgcccactacacacagaagagcctctccctgtctct |
| | | gggtaaa |
| 60 | Y32H heavy | caggttcagctggtgcagtctggagctgaggtgaagaagcctggggcctcagtgaaggtttcct |
| | chain with PLA | gcaaggcttctggctacatattcagtaaccattggatacagtgggtgcgacaggcccctggacaa |
| | mutation factor | ggccttgagtggatgggtgagattttacctggaagtggttctactgagtacactgagaacttcaag |
| | H fusion | gacagggtcaccatgactagggatacatccacgagcacagtctacatggagctcagcagcctg |
| | | agatcagaggacacggccgtctattactgtgcaagatatttcttcggtagtagccccaactggtac |
| | | ttcgatgtctggggccaggggaccctggtcaccgtctcctcagctagcaccaagggcccatcgg |
| | | tcttccccctggcgccctgctccaggagcacctccgagagcacagccgccctgggctgcctggt |
| | | caaggactacttccccgaaccggtgacggtgtcgtggaactcaggcgccctgaccagcggcgt |
| | | gcacaccttcccggctgtcctacagtcctcaggactctactccctcagcagcgtggtgaccgtgc |
| | | cctccagcagcttgggcacgaagacctacacctgcaacgtagatcacaagcccagcaacacca |
| | | aggtggacaagagagttgagtccaaatatggtcccccatgcccaccatgcccagcacctgagtt |
| | | cctggggggaccatcagtcttcctgttccccccaaaacccaaggacactctcatgatctcccgga |
| | | cccctgaggtcacgtgcgtggtggtggacgtgagccaggaagaccccgaggtccagttcaact |
| | | ggtacgtggatggcgtggaggtgcataatgccaagacaaagccgcgggaggagcagttcaac |
| | | agcacgtaccgtgtggtcagcgtcctcaccgtcctgcaccaggactggctgaacggcaaggag |
| | | tacaagtgcaaggtctccaacaaaggcctcccgtcctccategagaaaaccatctccaaagcca |
| | | aagggcagccccgagagccacaggtgtacaccctgcccccatcccaggaggagatgaccaa |
| | | gaaccaggtcagcctgacctgcctggtcaaaggcttctaccccagcgacatcgccgtggagtg |
| | | ggagagcaatgggcagccggagaacaactacaagaccacgcctcccgtgctggactccgacg |
| | | gctccttcttcctctacagcaggctcaccgtggacaagagcaggtggcaggaggggaatgtctt |
| | | ctcatgctccgtgctgcatgaggctctgcacgcccactacacacagaagagcctctccctgtctct |
| | | gggtaaagaggattgtaatgaactgcctccaagaaggaacacagaaattttgaccgggtcctggt |
| | | ccgatcaaacctaccctgaaggaactcaagctatatacaagtgcagacctggttatagatcactg |
| | | ggcaacattatcatggtatgtcgcaaaggagaatgggtggctctgaatcccctcagaaaatgcca |
| | | gaaacggccatgtggacaccccggcgataccccattcgggacatttaccttgactggaggcaat |
| | | gtattcgagtatggcgtgaaagctgtctatacctgtaacgaaggttaccaattgttgggagaaataa |
| | | attacagagaatgtgataccgatggatggaccaacgatattcccatatgcgaggttgttaagtgctt |
| | | gcctgtcactgcaccagaaaacgggaaaatcgtatctagcgcaatggagccagaccgcgaata |
| | | ccatttcgggcaggcagtgaggtttgtttgcaattccggttataagatagaaggggatgaggagat |
| | | gcactgtagcgacgatggtttttggtccaaggaaaagcccaagtgcgtcgaaataagttgcaagt |
| | | cacctgacgtcataaacgggagccccatatcccaaaagataatttacaaggagaacgaacgattt |
| | | caatataagtgtaatatggggtatgaatactccgagagaggtgatgccgtctgtaccgaaagtgg |
| | | atggcgaccactcccctcatgcgaagagaagtcctgtgataatccatacatccctaatggtgatta |
| | | ttccccccttcgaataaagcatcggacaggagacgagatcacatatcagtgtcgcaacggattct |
| | | atccagccaccagaggcaacactgcaaagtgtacatctacaggatggatacctgccccacgatg |
| | | taccttgaagccctga |
| 61 | Signal peptide | atgaaatggagctgggttattctcttcctcctgtcagtaactgcaggtgtccactcc |
| 62 | Signal peptide | atggaagccccagctcagcttctcttcctcctgctactctggctcccagataccaccgga |

1.-41. (canceled)

42. An isolated antibody that specifically binds to human C5 (anti-human C5 antibody), wherein the isolated anti-human C5 antibody comprises:
i) a heavy chain CDR1 (H-CDR1) comprising the amino acid sequence of SEQ ID NO:3, a heavy chain CDR2 (H-CDR2) comprising the amino acid sequence of SEQ ID NO:4, a heavy chain CDR3 (H-CDR3) comprising the amino acid sequence of SEQ ID NO:5, a light chain CDR1 (L-CDR1) comprising the amino acid sequence of SEQ ID NO:6, a light chain CDR2 (L-CDR2) comprising the amino acid sequence of SEQ ID NO:7, and a light chain CDR3 (L-CDR3) comprising the amino acid sequence of SEQ ID NO:8;
ii) an H-CDR1 comprising the amino acid sequence of SEQ ID NO:3, an H-CDR2 comprising the amino acid sequence of SEQ ID NO:4, an H-CDR3 comprising the amino acid sequence of SEQ ID NO:5, an L-CDR1 comprising the amino acid sequence of SEQ ID NO:11, an L-CDR2 comprising the amino acid sequence of SEQ ID NO:7, an L-CDR3 comprising the amino acid sequence of SEQ ID NO:8;
iii) an H-CDR1 comprising the amino acid sequence of SEQ ID NO:3, an H-CDR2 comprising the amino acid sequence of SEQ ID NO:4, an H-CDR3 comprising the amino acid sequence of SEQ ID NO:5, an L-CDR1 comprising the amino acid sequence of SEQ ID NO:12, an L-CDR2 comprising the amino acid sequence of SEQ ID NO:7, an L-CDR3 comprising the amino acid sequence of SEQ ID NO:8;
iv) an H-CDR1 comprising the amino acid sequence of SEQ ID NO:3, an H-CDR2 comprising the amino acid sequence of SEQ ID NO:4, an H-CDR3 comprising the amino acid sequence of SEQ ID NO:5, an L-CDR1 comprising the amino acid sequence of SEQ ID NO:13, an L-CDR2 comprising the amino acid sequence of SEQ ID NO:7, an L-CDR3 comprising the amino acid sequence of SEQ ID NO:8;
v) an H-CDR1 comprising the amino acid sequence of SEQ ID NO:3, an H-CDR2 comprising the amino acid sequence of SEQ ID NO:4, an H-CDR3 comprising the amino acid sequence of SEQ ID NO:5, an L-CDR1 comprising the amino acid sequence of SEQ ID NO:6, an L-CDR2 comprising the amino acid sequence of SEQ ID NO:14, an L-CDR3 comprising the amino acid sequence of SEQ ID NO:8;
vi) an H-CDR1 comprising the amino acid sequence of SEQ ID NO:3, an H-CDR2 comprising the amino acid sequence of SEQ ID NO:4, an H-CDR3 comprising the amino acid sequence of SEQ ID NO:5, an L-CDR1 comprising the amino acid sequence of SEQ ID NO:6, an L-CDR2 comprising the amino acid sequence of SEQ ID NO:15, an L-CDR3 comprising the amino acid sequence of SEQ ID NO:8;
vii) an H-CDR1 comprising the amino acid sequence of SEQ ID NO:3, an H-CDR2 comprising the amino acid sequence of SEQ ID NO:4, an H-CDR3 comprising the amino acid sequence of SEQ ID NO:5, an L-CDR1 comprising the amino acid sequence of SEQ ID NO:6, an L-CDR2 comprising the amino acid sequence of SEQ ID NO:16, an L-CDR3 comprising the amino acid sequence of SEQ ID NO:8; or
viii) an H-CDR1 comprising the amino acid sequence of SEQ ID NO:3, an H-CDR2 comprising the amino acid sequence of SEQ ID NO:4, an H-CDR3 comprising the amino acid sequence of SEQ ID NO:5, an L-CDR1 comprising the amino acid sequence of SEQ ID NO:6, an L-CDR2 comprising the amino acid sequence of SEQ ID NO:7, an L-CDR3 comprising the amino acid sequence of SEQ ID NO:17.

43. The isolated anti-human C5 antibody of claim 42, wherein the isolated anti-human C5 antibody comprises:
i) a VH comprising the amino acid sequence SEQ ID NO:9, or a variant thereof that is at least about 85% identical to SEQ ID NO:9 and a VL comprising the amino acid sequence of SEQ ID NO:2, or a variant thereof that is at least about 85% identical to SEQ ID NO:2;
ii) a VH comprising the amino acid sequence SEQ ID NO: 9, or a variant thereof that is at least about 85% identical to SEQ ID NO:9, and a VL comprising the amino acid sequence of SEQ ID NO:18, or a variant thereof that is at least about 85% identical to SEQ ID NO: 18;
iii) a VH comprising the amino acid sequence SEQ ID NO: 9, or a variant thereof that is at least about 85% identical to SEQ ID NO: 9, and a VL comprising the amino acid sequence of SEQ ID NO: 19, or a variant thereof that is at least about 85% identical to SEQ ID NO: 19;
iv) a VH comprising the amino acid sequence SEQ ID NO: 9, or a variant thereof that is at least about 85% identical to SEQ ID NO: 9, and a VL comprising the amino acid sequence of SEQ ID NO: 20, or a variant thereof that is at least about 85% identical to SEQ ID NO: 20;
v) a VH comprising the amino acid sequence SEQ ID NO: 9, or a variant thereof that is at least about 85% identical to SEQ ID NO: 9, and a VL comprising the amino acid sequence of SEQ ID NO: 21, or a variant thereof that is at least about 85% identical to SEQ ID NO: 21;
vi) a VH comprising the amino acid sequence SEQ ID NO: 9, or a variant thereof that is at least about 85% identical to SEQ ID NO: 9, and a VL comprising the amino acid sequence of SEQ ID NO: 22, or a variant thereof that is at least about 85% identical to SEQ ID NO: 22;
vii) a VH comprising the amino acid sequence SEQ ID NO: 9, or a variant thereof that is at least about 85% identical to SEQ ID NO: 9, and a VL comprising the amino acid sequence of SEQ ID NO: 23, or a variant thereof that is at least about 85% identical to SEQ ID NO: 23;
viii) a VH comprising the amino acid sequence SEQ ID NO: 9, or a variant thereof that is at least about 85% identical to SEQ ID NO: 9, and a VL comprising the amino acid sequence of SEQ ID NO: 24, or a variant thereof that is at least about 85% identical to SEQ ID NO: 24; or
ix) a VH comprising the amino acid sequence SEQ ID NO: 9, or a variant thereof that is at least about 85% identical to SEQ ID NO: 9, and a VL comprising the amino acid sequence of SEQ ID NO: 25, or a variant thereof that is at least about 85% identical to SEQ ID NO: 25.

44. The isolated anti-human C5 antibody of claim 43, wherein the isolated anti-human C5 antibody comprises a Q38R mutation in the VL, and wherein the mutation position is in reference to SEQ ID NO:2 under the AbM numbering system.

45. The isolated anti-human C5 antibody of claim 42, wherein the isolated anti-human C5 antibody is a full-length antibody, Fab, Fab′, F(ab)₂, F(ab′)₂, scFv, or a combination thereof.

46. The isolated anti-human C5 antibody of claim 45, wherein the isolated anti-human C5 antibody is a full-length antibody (anti-human C5 full-length antibody).

47. The isolated anti-human C5 antibody of claim 46, wherein the anti-human C5 full-length antibody comprises:
i) a heavy chain comprising the amino acid sequence of SEQ ID NO: 28, and a light chain comprising an VL comprising the amino acid sequence of SEQ ID NO: 23; or
ii) a heavy chain comprising the amino acid sequence of SEQ ID NO: 28, and a light chain comprising an VL comprising the amino acid sequence of SEQ ID NO: 18.

48. The isolated anti-human C5 antibody of claim 46, wherein the anti-human C5 full-length antibody comprises an Fc region, and wherein the Fc region comprises the amino acid residues 99-327 of SEQ ID NOs: 26 or 27.

49. A fusion protein comprising the isolated anti-human C5 antibody of claim 42 fused to a factor H polypeptide or fragment thereof.

50. The fusion protein of claim 49, wherein the factor H polypeptide or fragment thereof comprises short consensus repeat (SCR) domains 1-4 of factor H.

51. The fusion protein of claim 49, wherein the isolated anti-human C5 antibody is an anti-human C5 full-length antibody, and wherein the factor H polypeptide or fragment thereof is fused to the C-terminus of at least one of the heavy chains of the anti-human C5 full-length antibody.

52. The fusion protein of claim 51, wherein the fusion protein comprises a first factor H polypeptide or fragment thereof, a second factor H polypeptide or fragment thereof, and the anti-human C5 full-length antibody, wherein the first factor H polypeptide or fragment thereof is fused to the C-terminus of a first heavy chain of the anti-human C5 full-length antibody to form a first fusion polypeptide, and the second factor H polypeptide or fragment thereof is fused to the C-terminus of a second heavy chain of the anti-human C5 full-length antibody to form a second fusion polypeptide, wherein the first fusion polypeptide and the second fusion polypeptide each comprises the amino acid sequence of SEQ ID NOs: 10, and wherein each light chain of the anti-human C5 full-length antibody comprises a VL comprising the amino acid sequence of SEQ ID NO: 18 or 23.

53. An isolated nucleic acid encoding the isolated anti-human C5 antibody of claim 42.

54. A vector comprising the isolated nucleic acid of claim 53.

55. The vector of claim 54, wherein the vector is a viral vector.

56. A host cell comprising the vector of claim 54.

57. A method of producing an isolated anti-human C5 antibody, comprising culturing the host cell of claim 56 under a condition sufficient to allow expression of the isolated anti-human C5 antibody encoded by the vector.

58. A pharmaceutical composition comprising the isolated anti-human C5 antibody of claim 42 and a pharmaceutically acceptable carrier.

59. A method of treating an individual having a complement-associated disease or condition, comprising administering to the individual an effective amount of the pharmaceutical composition of claim 58.

60. A method of reducing the activity of a complement system in an individual, comprising administering to the individual an effective amount of the pharmaceutical composition of claim 58.

61. A method of treating an individual having a complement-associated disease or condition, comprising administering to the individual an effective amount of the viral vector of claim 55.


SEQUENCE LISTING (62 sequences)

SEQ ID NO: 1 - protein, 122 aa, Artificial Sequence [sequence omitted]

SEQ ID NO: 2 - protein, 107 aa, Artificial Sequence [sequence omitted]

SEQ ID NO: 3 - protein, 12 aa, Artificial Sequence
  H-CDR1 containing Y32H
ASGYIFSNHWIQ

SEQ ID NO: 4 - protein, 10 aa, Artificial Sequence
  H-CDR2 of anti-C5 mAb VH no mutation
EILPGSGSTE

SEQ ID NO: 5 - protein, 15 aa, Artificial Sequence
  H-CDR3 of anti-C5 mAb VH no mutation
ARYFFGSSPNWYFDV

SEQ ID NO: 6 - protein, 11 aa, Artificial Sequence
  L-CDR1 of anti-C5 mAb VL no mutation
GASENIYGALN

SEQ ID NO: 7 - protein, 8 aa, Artificial Sequence
  L-CDR2 of anti-C5 mAb VL no mutation
YGATNLAD

SEQ ID NO: 8 - protein, 9 aa, Artificial Sequence
  L-CDR3 of anti-C5 mAb VL no mutation
QNVLNTPLT

SEQ ID NO: 9 - protein, 122 aa, Artificial Sequence
  VH of Y32H mutant
QVQLVQSGAEVKKPGASVKVSCKASGYIFSNHWIQWVRQAPGQGLEWMGEILPGSGSTEYTENFKDRVTMTRDTSTSTVYMELSSLRSEDTAVYYCARYFFGSSPNWYFDVWGQGTLVTVSS

SEQ ID NO: 10 - protein, 1336 aa, Artificial Sequence
  VH of Y32H-IgG4PLA-fH1-5
QVQLVQSGAEVKKPGASVKVSCKASGYIFSNHWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHYTQKSLSLSLGKEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTQVQLVQSGAEVKKPGASVKVSCKASGYIFSNHWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHYTQKSLSLSLGKEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKP

SEQ ID NO: 11 - protein, 11 aa, Artificial Sequence
  L-CDR1 containing E27H
GASHNIYGALN

SEQ ID NO: 12 - protein, 11 aa, Artificial Sequence
  L-CDR1 containing N28H
GASEHIYGALN

SEQ ID NO: 13 - protein, 11 aa, Artificial Sequence
  L-CDR1 containing Y30H
GASENIHGALN

SEQ ID NO: 14 - protein, 8 aa, Artificial Sequence
  L-CDR2 containing T52H
YGAHNLAD

SEQ ID NO: 15 - protein, 8 aa, Artificial Sequence
  L-CDR2 containing L54H
YGATNHAD

SEQ ID NO: 16 - protein, 8 aa, Artificial Sequence
  L-CDR2 containing D56H
YGATNLAH

SEQ ID NO: 17 - protein, 9 aa, Artificial Sequence
  L-CDR3 containing T97H
QNVLNTPLH

SEQ ID NO: 18 - protein, 107 aa, Artificial Sequence [sequence omitted]

SEQ ID NO: 19 - protein, 107 aa, Artificial Sequence
  VL, Q38R/E27H
DIQMTQSPSSLSASVGDRVTITCGASHNIYGALNWYQRKPGKAPKLLIYGATNLADGVPSRFSGSGSGTDFTLTISSLQPEDFATYYCQNVLNTPLTFGQGTKVEIK

SEQ ID NO: 20 - protein, 107 aa, Artificial Sequence
  VL, Q38R/N28H
DIQMTQSPSSLSASVGDRVTITCGASEHIYGALNWYQRKPGKAPKLLIYGATNLADGVPSRFSGSGSGTDFTLTISSLQPEDFATYYCQNVLNTPLTFGQGTKVEIK

SEQ ID NO: 21 - protein, 107 aa, Artificial Sequence
  VL, Q38R/Y30H
DIQMTQSPSSLSASVGDRVTITCGASENIHGALNWYQRKPGKAPKLLIYGATNLADGVPSRFSGSGSGTDFTLTISSLQPEDFATYYCQNVLNTPLTFGQGTKVEIK

SEQ ID NO: 22 - protein, 107 aa, Artificial Sequence
  VL, Q38R/T52H
DIQMTQSPSSLSASVGDRVTITCGASENIYGALNWYQRKPGKAPKLLIYGAHNLADGVPSRFSGSGSGTDFTLTISSLQPEDFATYYCQNVLNTPLTFGQGTKVEIK

SEQ ID NO: 23 - protein, 107 aa, Artificial Sequence
  VL, Q38R/L54H
DIQMTQSPSSLSASVGDRVTITCGASENIYGALNWYQRKPGKAPKLLIYGATNHADGVPSRFSGSGSGTDFTLTISSLQPEDFATYYCQNVLNTPLTFGQGTKVEIK

SEQ ID NO: 24 - protein, 107 aa, Artificial Sequence
  VL, Q38R/D56H
DIQMTQSPSSLSASVGDRVTITCGASENIYGALNWYQRKPGKAPKLLIYGATNLAHGVPSRFSGSGSGTDFTLTISSLQPEDFATYYCQNVLNTPLTFGQGTKVEIK

SEQ ID NO: 25 - protein, 107 aa, Artificial Sequence
  VL, Q38R/T97H
DIQMTQSPSSLSASVGDRVTITCGASENIYGALNWYQRKPGKAPKLLIYGATNLADGVPSRFSGSGSGTDFTLTISSLQPEDFATYYCQNVLNTPLHFGQGTKVEIK

SEQ ID NO: 26 - protein, 327 aa, Artificial Sequence
  WT IgG4 Fc
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 327 aa, Artificial Sequence
  IgG4 Fc with PLA mutations
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHYTQKSLSLSLGK

SEQ ID NO: 28 - protein, 449 aa, Artificial Sequence
  Y32H heavy chain with PLA mutation
QVQLVQSGAEVKKPGASVKVSCKASGYIFSNHWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHYTQKSLSLSLGK

SEQ ID NO: 29 - protein, 755 aa, Artificial Sequence
  Y32H heavy chain with PLA mutation factor H fusion
QVQLVQSGAEVKKPGASVKVSCKASGYIFSNHWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHYTQKSLSLSLGKEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKP

SEQ ID NO: 30 - protein, 1676 aa, Artificial Sequence
  C5 (human)
MGLLGILCFLIFLGKTWGQEQTYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYDNGFLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVLPHFSVSIEPEYNFIGYKNFKNFEITIKARYFYNKVVTEADVYITFGIREDLKDDQKEMMQTAMQNTMLINGIAQVTFDSETAVKELSYYSLEDLNNKYLYIAVTVIESTGGFSEEAEIPGIKYVLSPYKLNLVATPLFLKPGIPYPIKVQVKDSLDQLVGGVPVTLNAQTIDVNQETSDLDPSKSVTRVDDGVASFVLNLPSGVTVLEFNVKTDAPDLPEENQAREGYRAIAYSSLSQSYLYIDWTDNHKALLVGEHLNIIVTPKSPYIDKITHYNYLILSKGKIIHFGTREKFSDASYQSINIPVTQNMVPSSRLLVYYIVTGEQTAELVSDSVWLNIEEKCGNQLQVHLSPDADAYSPGQTVSLNMATGMDSWVALAAVDSAVYGVQRGAKKPLERVFQFLEKSDLGCGAGGGLNNANVFHLAGLTFLTNANADDSQENDEPCKEILRPRRTLQKKIEEIAAKYKHSVVKKCCYDGACVNNDETCEQRAARISLGPRCIKAFTECCVVASQLRANISHKDMQLGRLHMKTLLPVSKPEIRSYFPESWLWEVHLVPRRKQLQFALPDSLTTWEIQGVGISNTGICVADTVKAKVFKDVFLEMNIPYSVVRGEQIQLKGTVYNYRTSGMQFCVKMSAVEGICTSESPVIDHQGTKSSKCVRQKVEGSSSHLVTFTVLPLEIGLHNINFSLETWFGKEILVKTLRVVPEGVKRESYSGVTLDPRGIYGTISRRKEFPYRIPLDLVPKTEIKRILSVKGLLVGEILSAVLSQEGINILTHLPKGSAEAELMSVVPVFYVFHYLETGNHWNIFHSDPLIEKQKLKKKLKEGMLSIMSYRNADYSYSVWKGGSASTWLTAFALRVLGQVNKYVEQNQNSICNSLLWLVENYQLDNGSFKENSQYQPIKLQGTLPVEARENSLYLTAFTVIGIRKAFDICPLVKIDTALIKADNFLLENTLPAQSTFTLAISAYALSLGDKTHPQFRSIVSALKREALVKGNPPIYRFWKDNLQHKDSSVPNTGTARMVETTAYALLTSLNLKDINYVNPVIKWLSEEQRYGGGFYSTQDTINAIEGLTEYSLLVKQLRLSMDIDVSYKHKGALHNYKMTDKNFLGRPVEVLLNDDLIVSTGFGSGLATVHVTTVVHKTSTSEEVCSFYLKIDTQDIEASHYRGYGNSDYKRIVACASYKPSREESSSGSSHAVMDISLPTGISANEEDLKALVEGVDQLFTDYQIKDGHVILQLNSIPSSDFLCVRFRIFELFEVGFLSPATFTVYEYHRPDKQCTMFYSTSNIKIQKVCEGAACKCVEADCGQMQEELDLTISAETRKQTACKPEIAYAYKVSITSITVENVFVKYKATLLDIYKTGEAVAEKDSEITFIKKVTCTNAELVKGRQYLIMGKEALQIKYNFSFRYIYPLDSLTWIEYWPRDTTCSSCQAFLANLDEFAEDIFLNGC

SEQ ID NO: 31 - protein, 19 aa, Artificial Sequence
  Signal peptide (1)
MKWSWVILFLLSVTAGVHS

SEQ ID NO: 32 - protein, 20 aa, Artificial Sequence
  Signal peptide (2)
MEAPAQLLFLLLLWLPDTTG

SEQ ID NO: 33 - DNA, 366 bp, Artificial Sequence
  Anti-C5 mAb VH no mutation - nucleic acid
caggttcagctggtgcagtctggagctgaggtgaagaagcctggggcctcagtgaaggtttcctgcaaggcttctggctacatattcagtaactactggatacagtgggtgcgacaggcccctggacaaggccttgagtggatgggtgagattttacctggaagtggttctactgagtacactgagaacttcaaggacagggtcaccatgactagggatacatccacgagcacagtctacatggagctcagcagcctgagatcagaggacacggccgtctattactgtgcaagatatttcttcggtagtagccccaactggtacttcgatgtctggggccaggggaccctggtcaccgtctcctca

SEQ ID NO: 34 - DNA, 321 bp, Artificial Sequence
  Anti-C5 mAb VL no mutation - nucleic acid
gacatccagatgactcagtctccatcttcactgtctgcatctgtgggagaccgggtcaccatcacatgtggagcaagtgagaatatttacggtgctttaaattggtatcagcagaaacctggaaaagcacctaagctcctgatctatggtgcaaccaacttggcagatggcgtcccttcgaggttcagtggcagtggatctggtaccgatttcacgctcactatcagtagcctgcagcctgaggatttcgcaacgtattactgtcaaaatgtgttaaatactcctctcacgttcggtcaggggaccaaggtggagatcaaa

SEQ ID NO: 35 - DNA, 30 bp, Artificial Sequence
  H-CDR1 containing Y32H - nucleic acid
ggctacatattcagtaaccattggatacag

SEQ ID NO: 36 - DNA, 30 bp, Artificial Sequence
  H-CDR2 of anti-C5 mAb VH no mutation - nucleic acid
gagattttacctggaagtggttctactgag

SEQ ID NO: 37 - DNA, 45 bp, Artificial Sequence
  H-CDR3 of anti-C5 mAb VH no mutation - nucleic acid
gcaagatatttcttcggtagtagccccaactggtacttcgatgtc

SEQ ID NO: 38 - DNA, 33 bp, Artificial Sequence
  L-CDR1 of anti-C5 mAb VL no mutation - nucleic acid
ggagcaagtgagaatatttacggtgctttaaat

SEQ ID NO: 39 - DNA, 24 bp, Artificial Sequence
  L-CDR2 of anti-C5 mAb VL no mutation - nucleic acid
tatggtgcaaccaacttggcagat

SEQ ID NO: 40 - DNA, 27 bp, Artificial Sequence
  L-CDR3 of anti-C5 mAb VL no mutation - nucleic acid
caaaatgtgttaaatactcctctcacg

SEQ ID NO: 41 - DNA, 366 bp, Artificial Sequence
  VH of Y32H mutant - nucleic acid
caggttcagctggtgcagtctggagctgaggtgaagaagcctggggcctcagtgaaggtttcctgcaaggcttctggctacatattcagtaaccattggatacagtgggtgcgacaggcccctggacaaggccttgagtggatgggtgagattttacctggaagtggttctactgagtacactgagaacttcaaggacagggtcaccatgactagggatacatccacgagcacagtctacatggagctcagcagcctgagatcagaggacacggccgtctattactgtgcaagatatttcttcggtagtagccccaactggtacttcgatgtctggggccaggggaccctggtcaccgtctcctca

SEQ ID NO: 42 - DNA, 2268 bp, Artificial Sequence
  VH of Y32H-IgG4PLA-fH1-5 - nucleic acid
caggttcagctggtgcagtctggagctgaggtgaagaagcctggggcctcagtgaaggtttcctgcaaggcttctggctacatattcagtaaccattggatacagtgggtgcgacaggcccctggacaaggccttgagtggatgggtgagattttacctggaagtggttctactgagtacactgagaacttcaaggacagggtcaccatgactagggatacatccacgagcacagtctacatggagctcagcagcctgagatcagaggacacggccgtctattactgtgcaagatatttcttcggtagtagccccaactggtacttcgatgtctggggccaggggaccctggtcaccgtctcctcagctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccttgccaccctcccaggaggagatgacaaaaaatcaggtaagtctcacatgcctggtcaaaggtttttacccatccgacattgctgtagagtgggaatccaacggtcagcccgagaacaactacaagacaactcccccagtcttggactcagatggctccttcttcttgtatagtagactgacagttgataaaagccgctggcaagaaggcaatgtctttagctgtagtgtcctgcatgaagcacttcacgcccattatacacaaaaatccctcagccttagcctagggaaggaggattgtaatgaactgcctccaagaaggaacacagaaattttgaccgggtcctggtccgatcaaacctaccctgaaggaactcaagctatatacaagtgcagacctggttatagatcactgggcaacattatcatggtatgtcgcaaaggagaatgggtggctctgaatcccctcagaaaatgccagaaacggccatgtggacaccccggcgataccccattcgggacatttaccttgactggaggcaatgtattcgagtatggcgtgaaagctgtctatacctgtaacgaaggttaccaattgttgggagaaataaattacagagaatgtgataccgatggatggaccaacgatattcccatatgcgaggttgttaagtgcttgcctgtcactgcaccagaaaacgggaaaatcgtatctagcgcaatggagccagaccgcgaataccatttcgggcaggcagtgaggtttgtttgcaattccggttataagatagaaggggatgaggagatgcactgtagcgacgatggtttttggtccaaggaaaagcccaagtgcgtcgaaataagttgcaagtcacctgacgtcataaacgggagccccatatcccaaaagataatttacaaggagaacgaacgatttcaatataagtgtaatatggggtatgaatactccgagagaggtgatgccgtctgtaccgaaagtggatggcgaccactcccctcatgcgaagagaagtcctgtgataatccatacatccctaatggtgattattccccccttcgaataaagcatcggacaggagacgagatcacatatcagtgtcgcaacggattctatccagccaccagaggcaacactgcaaagtgtacatctacaggatggatacctgccccacgatgtaccttgaagccctga

SEQ ID NO: 43 - DNA, 33 bp, Artificial Sequence
  L-CDR1 containing E27H - nucleic acid
ggagcaagtcataatatttacggtgctttaaat

SEQ ID NO: 44 - DNA, 33 bp, Artificial Sequence
  L-CDR1 containing N28H - nucleic acid
ggagcaagtgagcatatttacggtgctttaaat

SEQ ID NO: 45 - DNA, 33 bp, Artificial Sequence
  L-CDR1 containing Y30H - nucleic acid
ggagcaagtgagaatattcacggtgctttaaat

SEQ ID NO: 46 - DNA, 24 bp, Artificial Sequence
  L-CDR2 containing T52H - nucleic acid
tatggtgcacacaacttggcagat

SEQ ID NO: 47 - DNA, 24 bp, Artificial Sequence
  L-CDR2 containing L54H - nucleic acid
tatggtgcaaccaaccatgcagat

SEQ ID NO: 48 - DNA, 24 bp, Artificial Sequence
  L-CDR2 containing D56H - nucleic acid
tatggtgcaaccaacttggcacat

SEQ ID NO: 49 - DNA, 27 bp, Artificial Sequence
  L-CDR3 containg T97H - nucleic acid
caaaatgtgttaaatactcctctccac

SEQ ID NO: 50 - DNA, 321 bp, Artificial Sequence
  Kappa light chain, Q38R - nucleic acid
gacatccagatgactcagtctccatcttcactgtctgcatctgtgggagaccgggtcaccatcacatgtggagcaagtgagaatatttacggtgctttaaattggtatcagcggaaacctggaaaagcacctaagctcctgatctatggtgcaaccaacttggcagatggcgtcccttcgaggttcagtggcagtggatctggtaccgatttcacgctcactatcagtagcctgcagcctgaggatttcgcaacgtattactgtcaaaatgtgttaaatactcctctcacgttcggtcaggggaccaaggtggagatcaaa

SEQ ID NO: 51 - DNA, 321 bp, Artificial Sequence
  Kappa light chain, Q38R/E27H - nucleic acid
gacatccagatgactcagtctccatcttcactgtctgcatctgtgggagaccgggtcaccatcacatgtggagcaagtcataatatttacggtgctttaaattggtatcagcggaaacctggaaaagcacctaagctcctgatctatggtgcaaccaacttggcagatggcgtcccttcgaggttcagtggcagtggatctggtaccgatttcacgctcactatcagtagcctgcagcctgaggatttcgcaacgtattactgtcaaaatgtgttaaatactcctctcacgttcggtcaggggaccaaggtggagatcaaa

SEQ ID NO: 52 - DNA, 321 bp, Artificial Sequence
  Kappa light chain, Q38R/N28H - nucleic acid
gacatccagatgactcagtctccatcttcactgtctgcatctgtgggagaccgggtcaccatcacatgtggagcaagtgagcatatttacggtgctttaaattggtatcagcggaaacctggaaaagcacctaagctcctgatctatggtgcaaccaacttggcagatggcgtcccttcgaggttcagtggcagtggatctggtaccgatttcacgctcactatcagtagcctgcagcctgaggatttcgcaacgtattactgtcaaaatgtgttaaatactcctctcacgttcggtcaggggaccaaggtggagatcaaa

SEQ ID NO: 53 - DNA, 321 bp, Artificial Sequence
  Kappa light chain, Q38R/Y30H - nucleic acid
gacatccagatgactcagtctccatcttcactgtctgcatctgtgggagaccgggtcaccatcacatgtggagcaagtgagaatattcacggtgctttaaattggtatcagcggaaacctggaaaagcacctaagctcctgatctatggtgcaaccaacttggcagatggcgtcccttcgaggttcagtggcagtggatctggtaccgatttcacgctcactatcagtagcctgcagcctgaggatttcgcaacgtattactgtcaaaatgtgttaaatactcctctcacgttcggtcaggggaccaaggtggagatcaaa

SEQ ID NO: 54 - DNA, 321 bp, Artificial Sequence
  Kappa light chain, Q38R/T52H - nucleic acid
gacatccagatgactcagtctccatcttcactgtctgcatctgtgggagaccgggtcaccatcacatgtggagcaagtgagaatatttacggtgctttaaattggtatcagcggaaacctggaaaagcacctaagctcctgatctatggtgcacacaacttggcagatggcgtcccttcgaggttcagtggcagtggatctggtaccgatttcacgctcactatcagtagcctgcagcctgaggatttcgcaacgtattactgtcaaaatgtgttaaatactcctctcacgttcggtcaggggaccaaggtggagatcaaa

SEQ ID NO: 55 - DNA, 321 bp, Artificial Sequence
  Kappa light chain, Q38R/L54H - nucleic acid
gacatccagatgactcagtctccatcttcactgtctgcatctgtgggagaccgggtcaccatcacatgtggagcaagtgagaatatttacggtgctttaaattggtatcagcggaaacctggaaaagcacctaagctcctgatctatggtgcaaccaaccatgcagatggcgtcccttcgaggttcagtggcagtggatctggtaccgatttcacgctcactatcagtagcctgcagcctgaggatttcgcaacgtattactgtcaaaatgtgttaaatactcctctcacgttcggtcaggggaccaaggtggagatcaaa

SEQ ID NO: 56 - DNA, 321 bp, Artificial Sequence
  Kappa light chain, Q38R/D56H - nucleic acid
gacatccagatgactcagtctccatcttcactgtctgcatctgtgggagaccgggtcaccatcacatgtggagcaagtgagaatatttacggtgctttaaattggtatcagcggaaacctggaaaagcacctaagctcctgatctatggtgcaaccaacttggcacatggcgtcccttcgaggttcagtggcagtggatctggtaccgatttcacgctcactatcagtagcctgcagcctgaggatttcgcaacgtattactgtcaaaatgtgttaaatactcctctcacgttcggtcaggggaccaaggtggagatcaaa

SEQ ID NO: 57 - DNA, 321 bp, Artificial Sequence
  Kappa light chain, Q38R/T97H - nucleic acid
gacatccagatgactcagtctccatcttcactgtctgcatctgtgggagaccgggtcaccatcacatgtggagcaagtgagaatatttacggtgctttaaattggtatcagcggaaacctggaaaagcacctaagctcctgatctatggtgcaaccaacttggcagatggcgtcccttcgaggttcagtggcagtggatctggtaccgatttcacgctcactatcagtagcctgcagcctgaggatttcgcaacgtattactgtcaaaatgtgttaaatactcctctccacttcggtcaggggaccaaggtggagatcaaa

SEQ ID NO: 58 - DNA, 981 bp, Artificial Sequence
  IgG4 Fc with PLA mutations - nucleic acid
gctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgctgcatgaggctctgcacgcccactacacacagaagagcctctccctgtctctgggtaaa

SEQ ID NO: 59 - DNA, 1347 bp, Artificial Sequence
  Y32H heavy chain with PLA mutation - nucleic acid
caggttcagctggtgcagtctggagctgaggtgaagaagcctggggcctcagtgaaggtttcctgcaaggcttctggctacatattcagtaaccattggatacagtgggtgcgacaggcccctggacaaggccttgagtggatgggtgagattttacctggaagtggttctactgagtacactgagaacttcaaggacagggtcaccatgactagggatacatccacgagcacagtctacatggagctcagcagcctgagatcagaggacacggccgtctattactgtgcaagatatttcttcggtagtagccccaactggtacttcgatgtctggggccaggggaccctggtcaccgtctcctcagctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgctgcatgaggctctgcacgcccactacacacagaagagcctctccctgtctctgggtaaa

SEQ ID NO: 60 - DNA, 2268 bp, Artificial Sequence
  Y32H heavy chain with PLA mutation factor H fusion - nucleic acid
caggttcagctggtgcagtctggagctgaggtgaagaagcctggggcctcagtgaaggtttcctgcaaggcttctggctacatattcagtaaccattggatacagtgggtgcgacaggcccctggacaaggccttgagtggatgggtgagattttacctggaagtggttctactgagtacactgagaacttcaaggacagggtcaccatgactagggatacatccacgagcacagtctacatggagctcagcagcctgagatcagaggacacggccgtctattactgtgcaagatatttcttcggtagtagccccaactggtacttcgatgtctggggccaggggaccctggtcaccgtctcctcagctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgctgcatgaggctctgcacgcccactacacacagaagagcctctccctgtctctgggtaaagaggattgtaatgaactgcctccaagaaggaacacagaaattttgaccgggtcctggtccgatcaaacctaccctgaaggaactcaagctatatacaagtgcagacctggttatagatcactgggcaacattatcatggtatgtcgcaaaggagaatgggtggctctgaatcccctcagaaaatgccagaaacggccatgtggacaccccggcgataccccattcgggacatttaccttgactggaggcaatgtattcgagtatggcgtgaaagctgtctatacctgtaacgaaggttaccaattgttgggagaaataaattacagagaatgtgataccgatggatggaccaacgatattcccatatgcgaggttgttaagtgcttgcctgtcactgcaccagaaaacgggaaaatcgtatctagcgcaatggagccagaccgcgaataccatttcgggcaggcagtgaggtttgtttgcaattccggttataagatagaaggggatgaggagatgcactgtagcgacgatggtttttggtccaaggaaaagcccaagtgcgtcgaaataagttgcaagtcacctgacgtcataaacgggagccccatatcccaaaagataatttacaaggagaacgaacgatttcaatataagtgtaatatggggtatgaatactccgagagaggtgatgccgtctgtaccgaaagtggatggcgaccactcccctcatgcgaagagaagtcctgtgataatccatacatccctaatggtgattattccccccttcgaataaagcatcggacaggagacgagatcacatatcagtgtcgcaacggattctatccagccaccagaggcaacactgcaaagtgtacatctacaggatggatacctgccccacgatgtaccttgaagccctga

SEQ ID NO: 61 - DNA, 57 bp, Artificial Sequence
  Signal peptide (1) - nucleic acid
atgaaatggagctgggttattctcttcctcctgtcagtaactgcaggtgtccactcc

SEQ ID NO: 62 - DNA, 60 bp, Artificial Sequence
  Signal peptide (2) - nucleic acid
atggaagccccagctcagcttctcttcctcctgctactctggctcccagataccaccgga